(19) United States
(12) Patent Application Publication
(10) Pub. No.: US 2025/0034160 A1
(43) Pub. Date: Jan. 30, 2025

(54) Process For Preparing SHP2 Inhibitors

(71) Applicant: Genzyme Corporation, Cambridge, MA (US)

(72) Inventors: Fabio Morana, Novara (IT); Davide Gornati, Arluno (IT); Jacopo Roletto, Torino (IT)

(73) Assignee: Genzyme Corporation, Cambridge, MA (US)

(21) Appl. No.: 18/895,975

(22) Filed: Sep. 25, 2024

Related U.S. Application Data
(63) Continuation of application No. PCT/US23/16181, filed on Mar. 24, 2023.
(60) Provisional application No. 63/324,292, filed on Mar. 28, 2022.

(51) Int. Cl. C07D 491/107; C07D 211/22; C07D 213/73

(52) U.S. Cl. CPC C07D 491/107; C07D 211/22; C07D 213/73

(57) ABSTRACT

Provided herein are methods for preparing a SHP2 inhibitor and intermediates useful therein.

### CROSS-REFERENCE TO RELATED APPLICATIONS

This application is a continuation of International Application No. PCT/US2023/016181, filed on Mar. 24, 2023, which claims priority to U.S. Provisional Application No. 63/324,292, filed on Mar. 28, 2022, the disclosure of each of which is hereby incorporated by reference in its entirety.

### FIELD OF THE DISCLOSURE

The present disclosure relates generally to methods for preparing inhibitors of SHP2 and intermediates useful therein.

### BACKGROUND OF THE DISCLOSURE

SH2 domain-containing protein tyrosine phosphatase-2 (SHP2) is a non-receptor protein tyrosine phosphatase encoded by the PTPN11 gene that contributes to multiple cellular functions including proliferation, differentiation, cell cycle maintenance, and migration. SHP2 is involved in signaling through the Ras-mitogen-activated protein kinase, the JAK-STAT, or the phosphoinositol 3-kinase-AKT pathways.

SHP2 has two N-terminal Src homology 2 domains (N-SH2 and C-SH2), a catalytic domain (PTP), and a C-terminal tail. The two SH2 domains control the subcellular localization and functional regulation of SHP2. The protein exists in an inactive, self-inhibited conformation stabilized by a binding network involving residues from both the N-SH2 and PTP domains. Certain molecules, such as cytokines or growth factors, stimulate SHP2 and lead to exposure of the catalytic site, resulting in enzymatic activation of SHP2.

Mutations in the PTPN11 gene and subsequently in SHP2 have been identified in several human diseases, such as Noonan Syndrome, Leopard Syndrome, juvenile myelomonocytic leukemias, neuroblastoma, melanoma, acute myeloid leukemia, breast cancers, lung cancers, and colon cancer. As such, SHP2 is an attractive target for the development of novel therapies for the treatment of these diseases.

U.S. Pat. No. 10,590,090 discloses the compound {6-[(2-amino-3-chloropyridin-4-yl) sulfanyl]-3-[3S,4S)-4-amino-3-methyl-2-oxa-8-azaspiro[4.5]decan-8-yl]-5-methylpyrazin-2-yl}methanol (referred to in the present disclosure as the “compound of Formula (11)” or “Compound (11)”) as a SHP2 inhibitor. The synthesis of Compound (11) disclosed in U.S. Pat. No. 10,590,090 involves ten steps and requires chromatographic purification of diastereomers. Accordingly, a more efficient and selective synthesis of Compound (11) is desirable.

Accordingly, in one aspect, provided herein are improved methods for preparing Compound (11), or a salt thereof, and intermediates useful therein. In some embodiments, the compounds and/or salts of Formula (9) and Formula (10) (also referred to in the present disclosure as “Compound (9)” and “Compound (10)”, the chemical structures of which are shown below), are prepared without chromatographic purification, thus affording the desired product through a more efficient process.

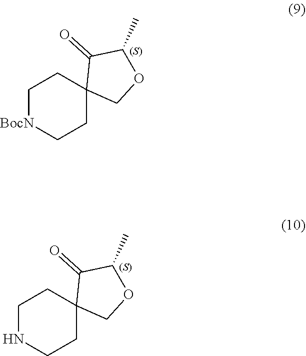

### SUMMARY OF THE DISCLOSURE

Described herein, in certain embodiments, are methods for preparing Compound (11), or a salt thereof, and intermediates useful therein, such as Compound (9) and Compound (10), or salts thereof.

The following embodiments are encompassed.

Embodiment 1. A method of preparing a compound of Formula (7):

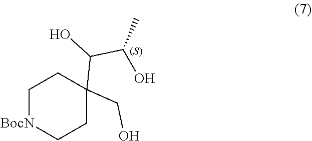

comprising:
reacting a compound of Formula (6):

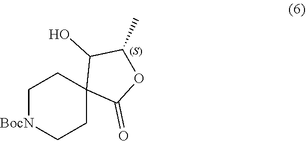

with a reducing agent to form the compound of Formula (7).

Embodiment 2. The method of embodiment 1, wherein the reducing agent is an organoaluminum hydride, an aluminum hydride, an organoborane hydride, or a borohydride reagent.

Embodiment 3. The method of embodiment 1 or 2, wherein the reaction is carried out using an alcohol, an aprotic solvent, or a mixture thereof as solvent.

Embodiment 4. A method of preparing a compound of Formula (6):

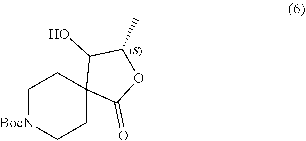

comprising reacting a compound of Formula (5):

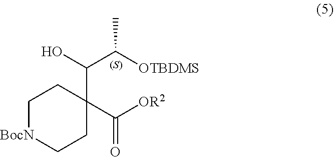

wherein R² is C₁-C₆ alkyl, and TBDMS is tert-butyldimethylsilyl ether, with a deprotecting agent to form the compound of Formula (6).

Embodiment 5. The method of any one of embodiments 1-4, wherein the compound of Formula (6) is prepared by reacting a compound of Formula (5):

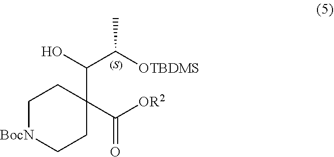

wherein R² is C₁-C₆ alkyl, and TBDMS is tert-butyldimethylsilyl ether, with a deprotecting agent to form the compound of Formula (6).

Embodiment 6. The method of embodiment 4 or 5, wherein the deprotecting agent is a source of fluoride ion, acetyl chloride, N-iodosuccinimide, HCl, acetic acid, formic acid, phosphoric acid, FeCl₃, AlCl₃, CeCl₃, oxalyl chloride, isobutyl chloroformate, ethyl chloroformate, or thionyl chloride.

Embodiment 7. The method of any one of embodiments 4-6, wherein the reaction of the compound of Formula (5) with the deprotecting agent is carried out using an aprotic solvent.

Embodiment 8. The method of any one of embodiments 4-7, wherein the compound of Formula (5) is prepared by reacting a compound of Formula (3):

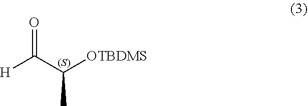

with a compound of Formula (4):

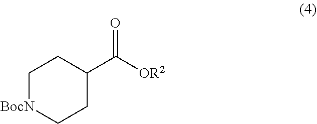

wherein R² is C₁-C₆ alkyl,
to form the compound of Formula (5).

Embodiment 9. The method of embodiment 8, wherein the reaction of the compound of Formula (3) with the compound of Formula (4) further comprises a base.

Embodiment 10. The method of embodiment 8 or 9, wherein the reaction of the compound of Formula (3) with the compound of Formula (4) is carried out using an aprotic solvent.

Embodiment 11. The method of any one of embodiments 8-10, wherein the compound of Formula (3) is prepared by reacting a compound of Formula (2):

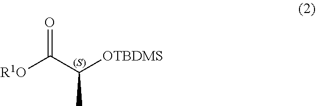

wherein R¹ is C₁-C₆ alkyl,
with a reducing agent to form the compound of Formula (3).

Embodiment 12. The method of embodiment 11, wherein the reducing agent used for the reaction of the compound of Formula (2) is an organoaluminum hydride, an aluminum hydride, an organoborane hydride, or a borohydride reagent.

Embodiment 13. The method of embodiment 11 or 12, wherein the reaction of the compound of Formula (2) with the reducing agent is carried out using an aprotic solvent.

Embodiment 14. The method of any one of embodiments 11-13, wherein the compound of Formula (2) is prepared by reacting a compound of Formula (1):

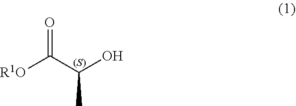

wherein R¹ is C₁-C₆ alkyl,
with TBDMS-X, wherein X is a halide,
to form the compound of Formula (2).

Embodiment 15. The method of embodiment 14, wherein the reaction of the compound of Formula (1) with TBDMS-X further comprises a base.

Embodiment 16. The method of embodiment 14 or 15, wherein the reaction of the compound of Formula (1) with TBDMS-X is carried out using an aprotic solvent.

Embodiment 17. A method of preparing a compound of Formula (8):

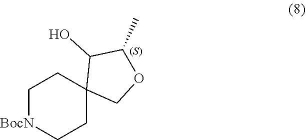

comprising reacting the compound of Formula (7) prepared according to the method of any one of embodiments 1-16 with a sulfonyl chloride or an acid chloride, and a base to form the compound of Formula (8).

Embodiment 18. The method of embodiment 17, wherein the sulfonyl chloride or the acid chloride is an arylsulfonyl chloride, an alkylsulfonyl chloride, or an aryl acid chloride.

Embodiment 19. The method of embodiment 17 or 18, wherein the reaction of Formula (7) with the sulfonyl chloride or the acid chloride and the base is carried out using a mixture of water and an aprotic solvent.

Embodiment 20. A method of preparing a compound of Formula (9):

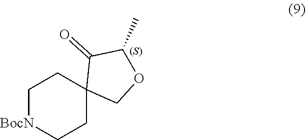

comprising reacting the compound of Formula (8) prepared according to the method of any one of embodiments 17-19 with an oxidizing agent to form the compound of Formula (9).

Embodiment 21. The method of embodiment 20, wherein the oxidizing agent is Dess-Martin periodinane, (2,2,6,6-tetramethylpiperidine-1-yl)oxyl (TEMPO), or sulfur trioxide pyridine complex.

Embodiment 22. The method of embodiment 21, wherein the reaction of the compound of Formula (8) with TEMPO further comprises (diacetoxyiodo)benzene or sodium hypochlorite, and optionally further comprises a salt.

Embodiment 23. The method of any one of embodiments 20-22, wherein the reaction of the compound of Formula (8) with the oxidizing agent is carried out using an aprotic solvent or a mixture of an aprotic solvent and water.

Embodiment 24. A method of preparing a compound of Formula (10) or a salt thereof:

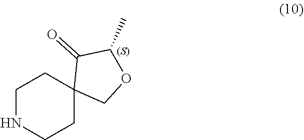

comprising reacting the compound of Formula (9) prepared according to the method of any one of embodiments 20-23 with an acid to form the compound of Formula (10) or a salt thereof.

Embodiment 25. A method of preparing a compound of Formula (10)⋅HCl comprising the following steps:

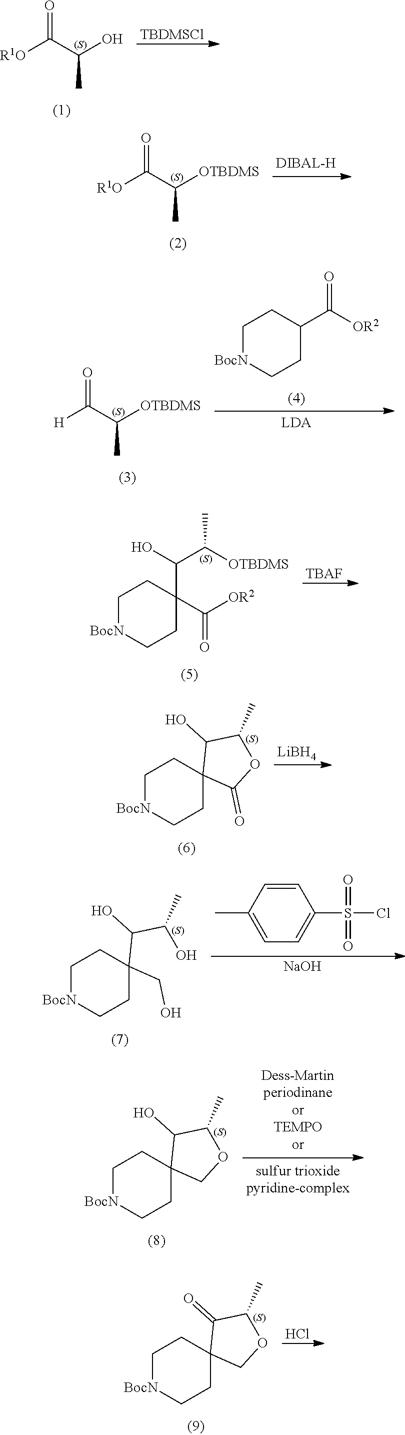

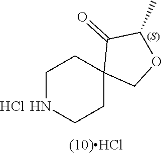

wherein R¹ and R² are each independently methyl or ethyl.

Embodiment 26. The method of any one of embodiments 20-25, wherein the compound of Formula (9) and/or the compound of Formula (10), or a salt thereof, are prepared without use of column chromatography.

Embodiment 27. A method of preparing a compound of Formula (11):

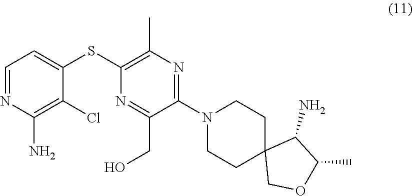 [SMILES: Cc1nc(N2CCC3(CC2)CO[C@@H](C)[C@H]3N)c(CO)nc1Sc1ccnc(N)c1Cl]

or a salt thereof, comprising converting the compound of Formula (10), or a salt thereof, prepared according to any one of embodiments 24-26 to the compound of Formula (11) or a salt thereof.

Embodiment 28. A compound of Formula (6):

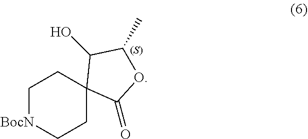

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the detailed descriptions are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the present disclosure.

As used herein, ranges and amounts can be expressed as “about” a particular value or range. About also includes the exact amount. Hence “about 0° C.” means “about 0° C.” and also “0° C.” Generally, the term “about” includes an amount that would be expected to be within experimental error, such as for example, within 15%, 10%, or 5%.

As used herein, the term “salt” refers to an acid or base salt of a compound disclosed herein. In some instances, the salt is a “pharmaceutically acceptable salt”, which is understood to be non-toxic. Non-limiting examples of salts include mineral acid (hydrochloric acid, hydrobromic acid, phosphoric acid, and the like) salts, organic acid (acetic acid, propionic acid, glutamic acid, citric acid, methanesulfonic acid, p-toluenesulfonic acid, and the like) salts, and quaternary ammonium (methyl iodide, ethyl iodide, and the like) salts.

Acid addition salts are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Non-limiting examples of inorganic salts include ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, non-limiting examples of which include ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins, and the like.

“Alkyl” refers to an unbranched or branched saturated hydrocarbon chain. As used herein, alkyl has 1 to 20 carbon atoms (i.e., C₁-C₂₀ alkyl), 1 to 10 carbon atoms (i.e., C₁-C₁₀ alkyl), 1 to 6 carbon atoms (i.e., C₁-C₆ alkyl) or 1 to 3 carbon atoms (i.e., C₁-C₃ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, 3-methylpentyl, 2-ethylhexyl, 3-ethylhexyl, and 4-ethylhexyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), isobutyl (i.e., —CH₂CH(CH₃)₂), sec-butyl (i.e., —CH(CH₃) CH₂CH₃), and tert-butyl (i.e., —C(CH₃)₃); and “propyl” includes n-propyl (i.e., —(CH₂)₂CH₃) and isopropyl (i.e., —CH(CH₃)₂).

“Halide,” “halogen,” or “halo” includes fluoro, chloro, bromo, and iodo.

As used herein, reference to a “compound of Formula (X)” is also meant to refer to “Compound (X)”. For example, “a compound of Formula (10)” is used interchangeably with “Compound (10)”.

“Therapeutically effective amount” of a compound or a composition refers to that amount of the compound or the composition that results in reduction or inhibition of symptoms or a prolongation of survival in a subject (i.e., a human patient). The results may require multiple doses of the compound or the composition.

“Treating” or “treatment” of a disease in a subject refers to 1) preventing the disease from occurring in a patient that is predisposed or does not yet display symptoms of the disease; 2) inhibiting the disease or arresting its development; or 3) ameliorating or causing regression of the disease. As used herein, “treatment” or “treating” is an approach for obtaining beneficial or desired results including clinical results. For the purposes of this disclosures, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a subject. Also encompassed by “treatment” is a reduction of pathological consequence of the disease or disorder. The methods of the invention contemplate any one or more of these aspects of treatment.

As used herein, the terms “subject(s)” and “patient(s)” mean any mammal. Examples include, but are not limited to, mice, rats, hamsters, guinea pigs, pigs, rabbits, cats, dogs, goats, sheep, cows, and humans. In some embodiments, the mammal is a human.

### Synthesis of Compounds

In one aspect, provided herein are methods of preparing a compound of Formula (10) or a salt thereof. Also provided herein are intermediate compounds useful for the preparation of a compound of Formula (10), or a salt thereof, as well as the synthesis of such intermediates. An overview of a synthesis of a compound of Formula (10), or a salt thereof, of the present disclosure is shown in Scheme 1.

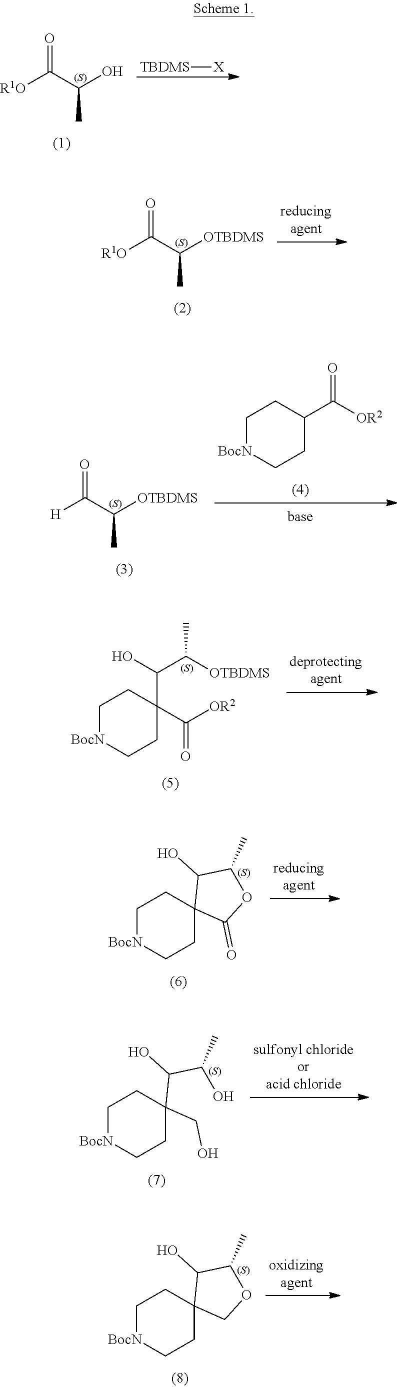

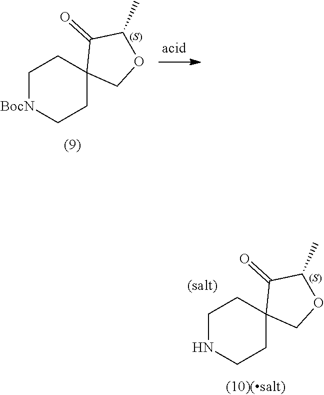

wherein X is halide; and R¹ and R² are each independently C₁-C₆ alkyl.

In some embodiments, provided herein are methods of preparing a compound of Formula (6). In some embodiments, provided herein is a method of preparing a compound of Formula (6) comprising reacting a compound of Formula (5) with a deprotecting agent. In some embodiments, provided herein is a method of preparing a compound of Formula (7) comprising reacting a compound of Formula (6) with a reducing agent. In some embodiments, provided herein is a method of preparing a compound of Formula (7) comprising reacting a compound of Formula (5) with a deprotecting agent to form a compound of Formula (6). In some embodiments, provided herein is a method of preparing a compound of Formula (8) comprising reacting a compound of Formula (6) with a reducing agent. In some embodiments, provided herein is a method of preparing a compound of Formula (8) comprising reacting a compound of Formula (5) with a deprotecting agent to form a compound of Formula (6). In some embodiments, provided herein is a method of preparing a compound of Formula (9) comprising reacting a compound of Formula (6) with a reducing agent. In some embodiments, provided herein is a method of preparing a compound of Formula (9) comprising reacting a compound of Formula (5) with a deprotecting agent to form a compound of Formula (6). In some embodiments, provided herein is a method of preparing a compound of Formula (10), or a salt thereof, comprising reacting a compound of Formula (6) with a reducing agent. In some embodiments, provided herein is a method of preparing a compound of Formula (10), or a salt thereof, comprising reacting a compound of Formula (5) with a deprotecting agent to form a compound of Formula (6).

In another aspect, provided herein are methods of preparing a compound of Formula (11) or a salt thereof. An overview of a synthesis of a compound of Formula (11), or a salt thereof, of the present disclosure is shown in Scheme 2.

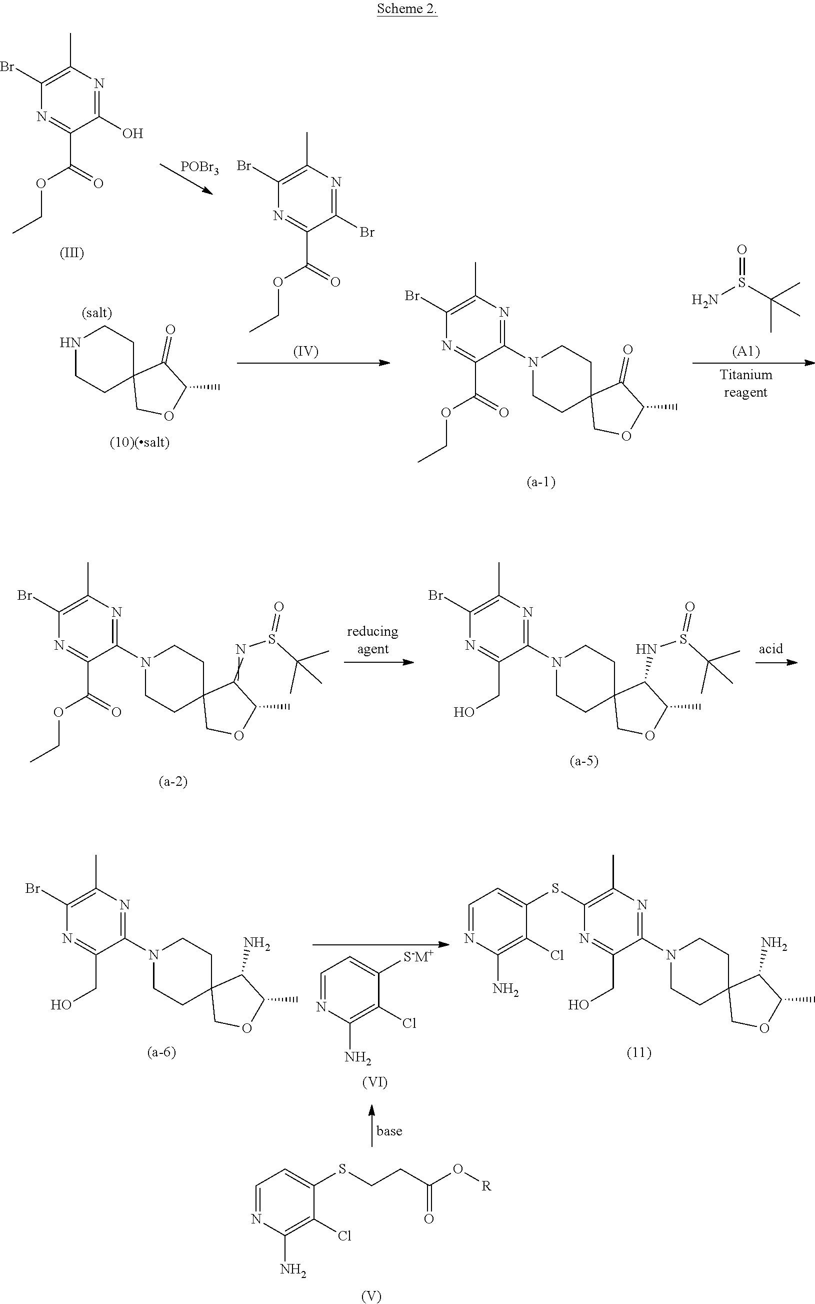

wherein M⁺ is Li⁺, Na⁺, or K⁺; R is C₁-C₁₂ alkyl; and [symbol] represents a double bond having either E or Z configuration. Throughout this description, a compound represented with [symbol] in its chemical structure indicates that the compound has a double bond in either E or Z configuration.

In some embodiments, [symbol] is a double bond having E configuration. In other embodiments, [symbol] is a double bond having Z configuration.

### Compound of Formula (7)

In one aspect, provided herein is a method of preparing a compound of Formula (7):

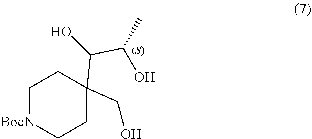

comprising reacting a compound of Formula (6):

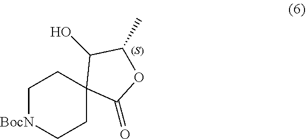

with a reducing agent to form the compound of Formula (7).

In some embodiments, the reducing agent is an organoaluminum hydride, an aluminum hydride, an organoborane hydride, or a borohydride reagent. In some embodiments, the reducing agent is an organoaluminum hydride. In some embodiments, the reducing agent is an aluminum hydride. In some embodiments, the reducing agent is an organoborane hydride. In some embodiments, the reducing agent is a borohydride reagent. In some embodiments, the reducing agent is diisobutylaluminum hydride (DIBAL-H), sodium bis(2-methoxyethoxy)aluminium hydride (Red-Al), LiAlH₄, LiBHEt₃, L-selectride, N-selectride, K-selectride, sodium borohydride, lithium borohydride, calcium borohydride, or potassium borohydride. In some embodiments, the reducing agent is lithium borohydride.

In some embodiments, the reaction is carried out using an alcohol, an aprotic solvent, or a mixture thereof as solvent. In some embodiments, the reaction is carried out using an alcohol. In some embodiments, the reaction is carried out using an aprotic solvent. In some embodiments, the reaction is carried out using a mixture of an alcohol and an aprotic solvent. In some embodiments, the alcohol is ethanol, methanol, or isopropanol. In some embodiments, the alcohol is ethanol. In some embodiments, the alcohol is methanol. In some embodiments, the aprotic solvent is an ether. In some embodiments, the ether is a cyclic ether. In some embodiments, the ether is a non-cyclic ether. In some embodiments, the aprotic solvent is an organic nitrile. In some embodiments, the aprotic solvent is tetrahydrofuran, 2-methyltetrahydrofuran, acetonitrile, methyl tert-butyl ether (MBTE), cyclopentylmethylether, or 1,4-dioxane. In some embodiments, the aprotic solvent is tetrahydrofuran. In some embodiments, the reaction is carried out using a mixture of an alcohol, such as methanol or ethanol, and tetrahydrofuran. In some embodiments, the reaction is carried out using a mixture of methanol and tetrahydrofuran. In some embodiments, the reaction is carried out using a mixture of ethanol and tetrahydrofuran.

In some embodiments, the reaction is carried out at a temperature of about 0 to 30° C. In some embodiments, the reaction is carried out at a temperature of about 10 to 20° C. In some embodiments, the reaction is carried out at a temperature of about 10° C., 15° C., or 20° C.

### Compound of Formula (6)

In another aspect, provided herein is a compound of Formula (6):

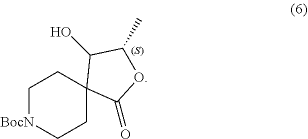

In some embodiments, the compound of Formula (6) is the compound of Formula (6a):

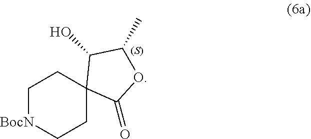

In some embodiments, the compound of Formula (6) is the compound of Formula (6b):

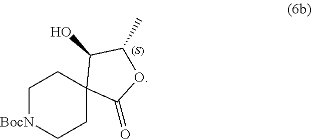

In another aspect, provided herein is a method of preparing a compound of Formula (6):

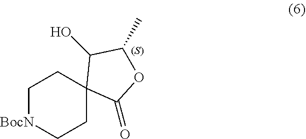

comprising reacting a compound of Formula (5):

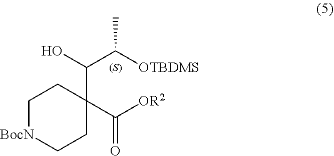

wherein R² is C₁-C₆ alkyl, and TBDMS is tert-butyldimethylsilyl ether,
with a deprotecting agent to form the compound of Formula (6).

In some embodiments, R² of the compound of Formula (5) is C₁-C₆ alkyl. In some embodiments, R² is C₁-C₃ alkyl. In some embodiments, R² is methyl, ethyl, isopropyl, or n-propyl. In some embodiments, R² is methyl or ethyl. In some embodiments, R² is methyl. In some embodiments, R² is ethyl.

In some embodiments, the deprotecting agent is a source of fluoride ion, acetyl chloride, N-iodosuccinimide, HCl, acetic acid, formic acid, phosphoric acid, FeCl₃, AlCl₃, CeCl₃, oxalyl chloride, isobutyl chloroformate, ethyl chloroformate, or thionyl chloride. In some embodiments, the deprotecting agent is an acyl chloride. In some embodiments, the deprotecting agent is an acid. In some embodiments, the deprotecting agent is an alkyl chloroformate. In some embodiments, the deprotecting agent is a metal halide salt, such as a metal chloride salt. In some embodiments, the deprotecting agent is a source of fluoride ion. In some embodiments, the source of fluoride ion is tetra-n-butylammonium fluoride (TBAF), NH₄F, CsF, HF pyridine, or HF·Et₃N. In some embodiments, the source of fluoride ion is tetra-n-butylammonium fluoride (TBAF).

In some embodiments, the reaction of the compound of Formula (5) with the deprotecting agent is carried out using an aprotic solvent. In some embodiments, the aprotic solvent is an ether. In some embodiments, the ether is a cyclic ether. In some embodiments, the ether is a non-cyclic ether. In some embodiments, the aprotic solvent is a halogenated hydrocarbon, such as a chlorinated hydrocarbon. In some embodiments, the aprotic solvent is a hydrocarbon, such as an aromatic hydrocarbon. In some embodiments, the aprotic solvent is an organic nitrile. In some embodiments, the aprotic solvent is tetrahydrofuran, 2-methyltetrahydrofuran, acetonitrile, 1,4-dioxane, toluene, dichloromethane, dichloroethane, chloroform, methyl tert-butyl ether (MBTE), cyclopentylmethylether, or a mixture thereof. In some embodiments, the aprotic solvent is tetrahydrofuran.

In some embodiments, the reaction of the compound of Formula (5) with the deprotecting agent is carried out at a temperature of about 0 to 25° C. In some embodiments, the reaction is carried out at a temperature of about 5 to 20° C. In some embodiments, the reaction is carried out at a temperature of about 10° C. In some embodiments, the reaction is carried out at a temperature of about 15° C.

In some embodiments, the compound of Formula (6) is isolated as a solid with high chemical purity. In some embodiments, the compound of Formula (6) is isolated as a solid with greater than about 90% chemical purity, such as about 90%, 91%, 92%, 93%, 94%, 95%, 96%, 97%, 98%, 99%, or 100% chemical purity. In some embodiments, the compound of Formula (6) is isolated as a solid with greater than about 99% chemical purity, such as about 99.1%, 99.2%, 99.3%, 99.4%, 99.5%, 99.6%, 99.7%, 99.8%, or 99.9% chemical purity. In some embodiments, the compound of Formula (6) is isolated as a solid with about 99% chemical purity.

In some embodiments, the compound of Formula (6) is isolated as a solid with high optical purity. In some embodiments, the compound of Formula (6) is isolated as a solid with greater than about 90% optical purity, such as about 90%, 91%, 92%, 93%, 94%, 95%, 96%, 97%, 98%, 99%, or 100% optical purity. In some embodiments, the compound of Formula (6) is isolated as a solid with greater than about 99% optical purity, such as about 99.1%, 99.2%, 99.3%, 99.4%, 99.5%, 99.6%, 99.7%, 99.8%, or 99.9% optical purity. In some embodiments, the compound of Formula (6) is isolated as a solid with about 99% optical purity.

In some embodiments, the high chemical and optical purity of the isolated compound of Formula (6) allows for the preparation of the compounds of Formula (9) and Formula (10), and salts thereof, without use of column chromatography (i.e., without chromatographic purification or chromatographic separation).

In some embodiments, the compound of Formula (6) is purified by simple crystallization after conventional work-up. In some embodiments, the crystallization of the compound of Formula (6) allows for removal of unreacted starting materials and impurities generated in previous steps of the synthesis, such as impurities formed during the synthesis of the compounds of Formula (2), (3), or (5). In some embodiments, the crystallization of the compound of Formula (6) allows for removal of any residual (R) enantiomer present in the commercially available starting material of the compound of Formula (1), which is typically between 0.7 and 2.5% wherein R¹ is ethyl. In some embodiments, preparation of the compound of Formula (6) allows for the synthesis of the compound of Formula (9) with an optical purity of greater than or equal to 98:2. In comparison, WO 2020/065453 discloses the preparation of the compound of Formula (9) with a lower optical purity (97:3).

### Compound of Formula (5)

In a further aspect, provided herein is a method of preparing a compound of Formula (5) comprising reacting a compound of Formula (3):

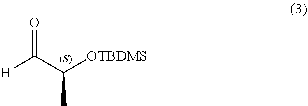

with a compound of Formula (4):

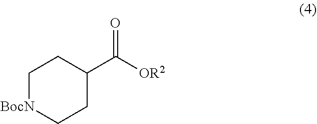

wherein R² is C₁-C₆ alkyl,
to form the compound of Formula (5).

In some embodiments, R² of the compound of Formula (4) is C₁-C₆ alkyl. In some embodiments, R² is C₁-C₃ alkyl. In some embodiments, R² is methyl, ethyl, isopropyl, or n-propyl. In some embodiments, R² is methyl or ethyl. In some embodiments, R² is methyl. In some embodiments, R² is ethyl.

In some embodiments, the reaction of the compound of Formula (3) with the compound of Formula (4) further comprises a base. In some embodiments, the base is a dialkylamide. In some embodiments, the base is lithium diisopropylamide (LDA), lithium bis(trimethylsilyl)amide (LiHMDS), lithium tetramethylpiperidide (LiTMP), sodium bis(trimethylsilyl)amide (NaHMDS), or potassium bis(trimethylsilyl)amide (KHMDS). In some embodiments, the base is lithium diisopropylamide (LDA).

In some embodiments, the reaction of the compound of Formula (3) with the compound of Formula (4) is carried out using an aprotic solvent. In some embodiments, the aprotic solvent is an ether. In some embodiments, the ether is a cyclic ether. In some embodiments, the ether is a non-cyclic ether. In some embodiments, the aprotic solvent is a halogenated hydrocarbon, such as a chlorinated hydrocarbon. In some embodiments, the aprotic solvent is an organic nitrile. In some embodiments, the aprotic solvent is tetrahydrofuran, 2-methyltetrahydrofuran, acetonitrile, 1,4-dioxane, toluene, dichloromethane, dichloroethane, chloroform, or a mixture thereof. In some embodiments, the aprotic solvent is tetrahydrofuran.

In some embodiments, the reaction of the compound of Formula (3) with the compound of Formula (4) is carried out at a temperature of about 0 to −40° C. In some embodiments, the reaction is carried out at a temperature of about −10 to −30° C. In some embodiments, the reaction is carried out at a temperature of about −15° C. In some embodiments, the reaction is carried out at a temperature of about −20° C.

### Compound of Formula (3)

In another aspect, provided herein is a method of preparing a compound of Formula (3) comprising reacting a compound of Formula (2):

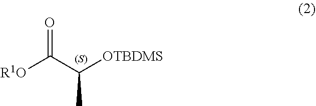

wherein R¹ is C₁-C₆ alkyl,
with a reducing agent to form the compound of Formula (3).

In some embodiments, R¹ of the compound of Formula (2) is C₁-C₆ alkyl. In some embodiments, R¹ is C₁-C₃ alkyl. In some embodiments, R¹ is methyl, ethyl, isopropyl, or n-propyl. In some embodiments, R¹ is methyl or ethyl. In some embodiments, R¹ is methyl. In some embodiments, R¹ is ethyl.

In some embodiments, the reducing agent used for the reaction of the compound of Formula (2) is an organoaluminum hydride, an aluminum hydride, an organoborane hydride, or a borohydride reagent. In some embodiments, the reducing agent is an organoaluminum hydride. In some embodiments, the reducing agent is an aluminum hydride. In some embodiments, the reducing agent is an organoborane hydride. In some embodiments, the reducing agent is a borohydride reagent. In some embodiments, the reducing agent is diisobutylaluminum hydride (DIBAL-H), sodium bis(2-methoxyethoxy)aluminium hydride (Red-Al), LiAlH₄, LiBHEt₃, L-selectride, N-selectride, K-selectride, sodium borohydride, lithium borohydride, calcium borohydride, or potassium borohydride. In some embodiments, the reducing agent is diisobutylaluminum hydride (DIBAL-H).

In some embodiments, the reaction of the compound of Formula (2) with the reducing agent is carried out using an aprotic solvent. In some embodiments, the aprotic solvent is an ether. In some embodiments, the ether is a cyclic ether. In some embodiments, the ether is a non-cyclic ether. In some embodiments, the aprotic solvent is a halogenated hydrocarbon, such as a chlorinated hydrocarbon. In some embodiments, the aprotic solvent is a hydrocarbon, such as a linear alkane or a cycloalkane. In some embodiments, the aprotic solvent is an organic nitrile. In some embodiments, the aprotic solvent is toluene, methyl tert-butyl ether (MBTE), cyclopentylmethylether, tetrahydrofuran, 2-methyltetrahydrofuran, acetonitrile, 1,4-dioxane, dichloromethane, dichloroethane, chloroform, n-hexane, cyclohexane, or a mixture thereof. In some embodiments, the aprotic solvent is toluene. In some embodiments, the aprotic solvent is a mixture of a cyclic ether and toluene. In some embodiments, the aprotic solvent is a mixture of a non-cyclic ether and toluene. In some embodiments, the aprotic solvent is methyl tert-butyl ether (MBTE). In some embodiments, the aprotic solvent is a mixture of toluene and methyl tert-butyl ether (MBTE). In some embodiments, the aprotic solvent is toluene, methyl tert-butyl ether (MBTE), or a mixture thereof.

In some embodiments, the reaction of the compound of Formula (2) with the reducing agent is carried out at a temperature of about −20 to −80° C. In some embodiments, the reaction is carried out at a temperature of about −30 to −60° C. In some embodiments, the reaction is carried out at a temperature of about −30° C., −40° C., −50° C., or −60° C. In some embodiments, the reaction is carried out at a temperature of about −40° C. or −50° C.

In some embodiments, the reaction of the compound of Formula (2) with the reducing agent comprises use of continuous stirred tank reactor (CSTR) technology. In some embodiments, use of CSTR technology for the reaction of the compound of Formula (2) with the reducing agent allows for low temperature reaction suitable for large-scale manufacturing.

### Compound of Formula (2)

In another aspect, provided herein is a method of preparing a compound of Formula (2) comprising reacting a compound of Formula (1):

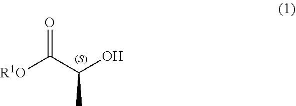

wherein R¹ is C₁-C₆ alkyl,
with TBDMS-X, wherein X is a halide,
to form the compound of Formula (2).

In some embodiments, R¹ of the compound of Formula (1) is C₁-C₆ alkyl. In some embodiments, R¹ is C₁-C₃ alkyl. In some embodiments, R¹ is methyl, ethyl, isopropyl, or n-propyl. In some embodiments, R¹ is methyl or ethyl. In some embodiments, R¹ is methyl. In some embodiments, R¹ is ethyl.

In some embodiments, X is Cl, Br, or I. In some embodiments, X is Cl or Br. In some embodiments, X is Cl. In some embodiments, X is Br.

In some embodiments, the reaction of the compound of Formula (1) with TBDMS-X further comprises a base. In some embodiments, the base is NaOH, KOH, LiOH, NaHCO₃, K₂CO₃, or imidazole. In some embodiments, the base is imidazole. In some embodiments, the base is NaOH, KOH, or LiOH. In some embodiments, the base is NaHCO₃ or K₂CO₃.

In some embodiments, the reaction of the compound of Formula (1) with TBDMS-X is carried out using an aprotic solvent. In some embodiments, the aprotic solvent is an ether. In some embodiments, the ether is a cyclic ether. In some embodiments, the ether is a non-cyclic ether. In some embodiments, the aprotic solvent is a halogenated hydrocarbon, such as a chlorinated hydrocarbon. In some embodiments, the aprotic solvent is a hydrocarbon, such as an aromatic hydrocarbon. In some embodiments, the aprotic solvent is an organic nitrile. In some embodiments, the aprotic solvent is tetrahydrofuran, 2-methyltetrahydrofuran, acetonitrile, 1,4-dioxane, toluene, dichloromethane, dichloroethane, chloroform, or a mixture thereof. In some embodiments, the aprotic solvent is toluene.

In some embodiments, the reaction of the compound of Formula (1) with TBDMS-X is carried out at a temperature of about −10 to 50° C. In some embodiments, the reaction is carried out at a temperature of about 0 to 25° C. In some embodiments, the reaction is carried out at a temperature of about 10° C., 15° C., or 20° C. In some embodiments, the reaction is carried out at a temperature of about 25° C., 30° C., 35° C., 40° C., or 45° C.

### Compound of Formula (8)

In another aspect, provided herein is a method of preparing a compound of Formula (8):

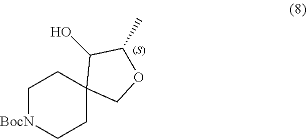

comprising reacting the compound of Formula (7) with a sulfonyl chloride or an acid chloride, and a base to form the compound of Formula (8).

In some embodiments, the reaction of the compound of Formula (7) is carried out using a sulfonyl chloride. In some embodiments, the reaction is carried out using an acid chloride. In some embodiments, the sulfonyl chloride or the acid chloride is an arylsulfonyl chloride, an alkylsulfonyl chloride, or an aryl acid chloride. In some embodiments, the sulfonyl chloride or the acid chloride is p-toluenesulfonylchloride, methanesulfonyl chloride, 4-bromo-benzenesulfonyl chloride, 4-nitro-benzenesulfonyl chloride, 1-naphthoyl chloride, or 2-naphthoyl chloride. In some embodiments, the sulfonyl chloride is p-toluenesulfonylchloride.

In some embodiments, the base used for the reaction of the compound of Formula (7) with the sulfonyl chloride or the acid chloride is NaOH, KOH, LiOH, Ca(OH) 2, CsOH, NaHCO₃, K₂CO₃, or Cs₂CO₃. In some embodiments, the base is NaOH, KOH, LiOH, Ca(OH) 2, or CsOH. In some embodiments, the base is NaOH. In some embodiments, the base is NaHCO₃, K₂CO₃, or Cs₂CO₃. In some embodiments, the mild base used for the reaction of the compound of Formula (7) with the sulfonyl chloride or the acid chloride is suitable for large-scale manufacturing.

In some embodiments, the reaction of the compound of Formula (7) with the sulfonyl chloride or the acid chloride is carried out using a mixture of water and an aprotic solvent. In some embodiments, the reaction is carried out using a biphasic solvent system. In some embodiments, the aprotic solvent is an ether. In some embodiments, the ether is a cyclic ether. In some embodiments, the ether is a non-cyclic ether. In some embodiments, the aprotic solvent is a hydrocarbon, such as an alkane, cycloalkane, or an aromatic hydrocarbon. In some embodiments, the aprotic solvent is a halogenated hydrocarbon, such as a chlorinated hydrocarbon. In some embodiments, the aprotic solvent is methyl tert-butyl ether (MBTE), toluene, cyclohexane, n-heptane, diisopropylether, cyclopentylmethylether, tetrahydrofuran, 2-methyltetrahydrofuran, acetonitrile, 1,4-dioxane, dichloromethane, dichloroethane, chloroform, or a mixture thereof. In some embodiments, the aprotic solvent is methyl tert-butyl ether (MBTE), toluene, cyclohexane, n-heptane, diisopropylether, or cyclopentylmethylether. In some embodiments, the aprotic solvent is methyl tert-butyl ether (MBTE). In some embodiments, the aprotic solvent is toluene. In some embodiments, the aprotic solvent is cyclohexane. In some embodiments, the aprotic solvent is n-heptane. In some embodiments, the aprotic solvent is diisopropylether. In some embodiments, the aprotic solvent is cyclopentylmethylether.

In some embodiments, the reaction of the compound of Formula (7) with the sulfonyl chloride or the acid chloride is carried out at a temperature of about −10 to 30° C. In some embodiments, the reaction is carried out at a temperature of about −5 to 25° C. In some embodiments, the reaction is carried out at a temperature of about 0° C. In some embodiments, the reaction is carried out at a temperature of about 10° C. or 20° C.

In some variations, wherein the compound of Formula (7) is reacted with p-toluenesulfonylchloride in a biphasic solvent system, the mild reaction conditions allow for the preparation of the compound of Formula (8) in high yield and with high enantiomeric ratio (such as greater than about 98:2) without use of column chromatography.

### Compound of Formula (9)

In a further aspect, provided herein is a method of preparing a compound of Formula (9):

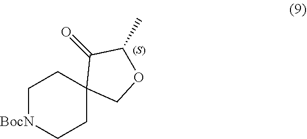

comprising reacting the compound of Formula (8) with an oxidizing agent to form the compound of Formula (9).

In some embodiments, the oxidizing agent is Dess-Martin periodinane. In some embodiments, the oxidizing agent is (2,2,6,6-tetramethylpiperidine-1-yl)oxyl (TEMPO). In some embodiments, the oxidizing agent is sulfur trioxide pyridine complex. In some embodiments, the safety and cost of the oxidizing agent allow for large-scale manufacturing. In some embodiments, the oxidizing agent allows for the preparation of the compound of Formula (9) without racemization at the stereocenter.

In some embodiments, wherein the oxidizing agent is TEMPO, the reaction further comprises (diacetoxyiodo)benzene or sodium hypochlorite. In some embodiments, wherein the oxidizing agent is TEMPO, the reaction further comprises a salt. In some embodiments, the salt is a metal halide salt. In some embodiments, the salt is a potassium halide salt. In some embodiments, the salt is KBr, KCl, or KI. In some embodiments, the salt is KBr.

In some embodiments, the reaction of the compound of Formula (8) with the oxidizing agent is carried out using an aprotic solvent or a mixture of an aprotic solvent and water. In some embodiments, the reaction is carried out using an aprotic solvent. In some embodiments, the reaction is carried out using a biphasic solvent system. In some embodiments, the reaction is carried out using a mixture of the aprotic solvent and water. In some embodiments, the aprotic solvent is an ether. In some embodiments, the ether is a cyclic ether. In some embodiments, the ether is a non-cyclic ether. In some embodiments, the aprotic solvent is a halogenated hydrocarbon, such as a chlorinated hydrocarbon. In some embodiments, the aprotic solvent is an organic nitrile. In some embodiments, the aprotic solvent is tetrahydrofuran, 2-methyltetrahydrofuran, acetonitrile, 1,4-dioxane, dichloromethane, dichloroethane, chloroform, or a mixture thereof. In some embodiments, the aprotic solvent is dichloromethane or acetonitrile. In some embodiments, the aprotic solvent is dichloromethane. In some embodiments, the aprotic solvent is acetonitrile. In some embodiments, the aprotic solvent is a mixture of water and dichloromethane. In some embodiments, the aprotic solvent is a mixture of water and acetonitrile.

In some embodiments, the reaction of the compound of Formula (8) with the oxidizing agent is carried out at a temperature of about −10 to 30° C. In some embodiments, the reaction is carried out at a temperature of about 0 to 25° C. In some embodiments, the reaction is carried out at a temperature of about 10° C., 15° C., or 20° C.

In some embodiments, the compound of Formula (9) is prepared without use of column chromatography. In some embodiments, the preparation of the compound of Formula (9) without use of column chromatography is due at least in part to the use of the intermediate compound of Formula (6).

### Compound of Formula (10)

Also provided herein is a method of preparing a compound of Formula (10) or a salt thereof:

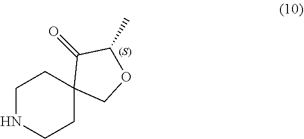

comprising reacting the compound of Formula (9) with an acid to form the compound of Formula (10) or a salt thereof.

In some embodiments, the acid is HCl, oxalic acid, phosphoric acid, trifluoroacetic acid, formic acid, HBr, or methanesulfonic acid. In some embodiments, the acid is HCl. In some embodiments, the acid is H₂SO₄.

In some embodiments, the compound of Formula (10) is provided as a salt. In some embodiments, the salt of the compound of Formula (10) is an HCl, oxalic acid, phosphoric acid, trifluoroacetic acid, formic acid, HBr, or methanesulfonic acid salt. In some embodiments, the salt of the compound of Formula (10) is an HCl salt. In some embodiments, the salt of the compound of Formula (10) is an H₂SO₄ salt.

In some embodiments, the reaction of the compound of Formula (9) with the acid is carried out using an aprotic solvent or a mixture of an aprotic solvent and a protic solvent. In some embodiments, the reaction is carried out using an aprotic solvent. In some embodiments, the reaction is carried out using a mixture of an aprotic solvent and a protic solvent. In some embodiments, the aprotic solvent is an organic nitrile. In some embodiments, the aprotic solvent is an ether. In some embodiments, the ether is a cyclic ether. In some embodiments, the ether is a non-cyclic ether. In some embodiments, the aprotic solvent is a ketone. In some embodiments, the aprotic solvent is acetone, acetonitrile, tetrahydrofuran, 2-methyltetrahydrofuran, 1,4-dioxane, or a mixture thereof. In some embodiments, the aprotic solvent is acetone. In some embodiments, the protic solvent is an alcohol. In some embodiments, the reaction is carried out using a mixture of acetone and an alcohol. In some embodiments, the alcohol is methanol, ethanol, isopropanol, or a mixture thereof. In some embodiments, the alcohol is methanol. In some embodiments, the alcohol is ethanol. In some embodiments, the alcohol is isopropanol. In some embodiments, the reaction is carried out using a mixture of acetone and methanol. In some embodiments, the reaction is carried out using a mixture of acetone and ethanol. In some embodiments, the reaction is carried out using a mixture of acetone and isopropanol.

In some embodiments, the reaction of the compound of Formula (9) with the acid is carried out at a temperature of about −10 to 30° C. In some embodiments, the reaction is carried out at a temperature of about 0 to 25° C. In some embodiments, the reaction is carried out at a temperature of about 10° C., 15° C., or 20° C.

In some embodiments, the compound of Formula (10), or a salt thereof, is prepared without use of column chromatography. In some embodiments, the preparation of the compound of Formula (10), or a salt thereof, without use of column chromatography is due at least in part to the use of the intermediate compound of Formula (6). In some embodiments, the compound of Formula (10), or a salt thereof, is generated and directly used in situ.

In some embodiments, the compound of Formula (10), or a salt thereof, is isolated as a solid with high chemical purity. In some embodiments, the compound of Formula (10), or a salt thereof, is isolated as a solid with greater than about 90% chemical purity, such as about 90%, 91%, 92%, 93%, 94%, 95%, 96%, 97%, 98%, 99%, or 100% chemical purity. In some embodiments, the compound of Formula (10), or a salt thereof, is isolated as a solid with greater than about 99% chemical purity, such as about 99.1%, 99.2%, 99.3%, 99.4%, 99.5%, 99.6%, 99.7%, 99.8%, or 99.9% chemical purity. In some embodiments, the compound of Formula (10), or a salt thereof, is isolated as a solid with about 99% chemical purity.

In some embodiments, the compound of Formula (10), or a salt thereof, is isolated as a solid with high optical purity. In some embodiments, the compound of Formula (10), or a salt thereof, is isolated as a solid with greater than about 90% optical purity, such as about 90%, 91%, 92%, 93%, 94%, 95%, 96%, 97%, 98%, 99%, or 100% optical purity. In some embodiments, the compound of Formula (10), or a salt thereof, is isolated as a solid with greater than about 99% optical purity, such as about 99.1%, 99.2%, 99.3%, 99.4%, 99.5%, 99.6%, 99.7%, 99.8%, or 99.9% optical purity. In some embodiments, the compound of Formula (10), or a salt thereof, is isolated as a solid with about 99% optical purity.

In another aspect, provided herein is a method of preparing a compound of Formula (10). HCl comprising the steps outlined in Scheme 3.

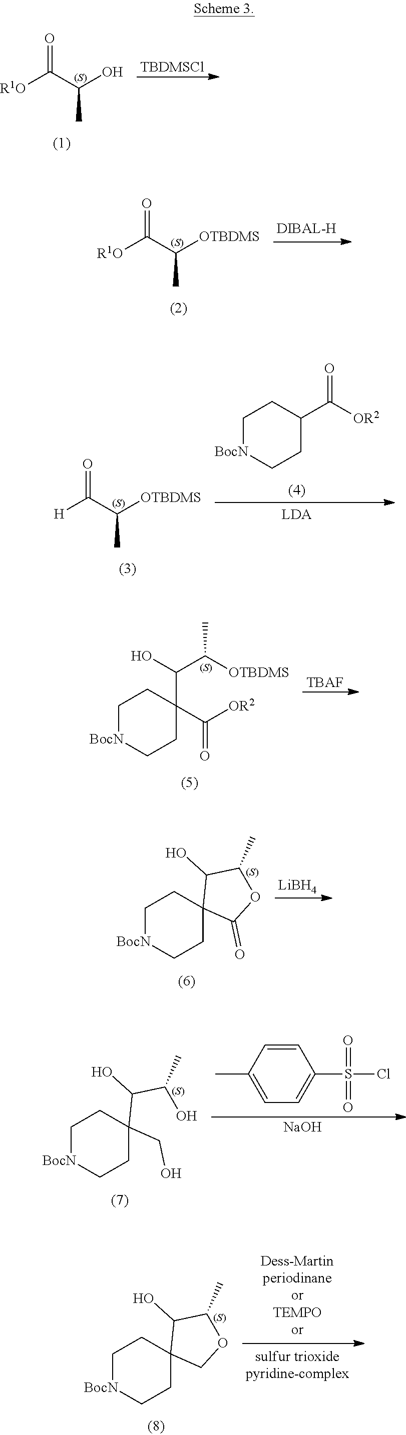

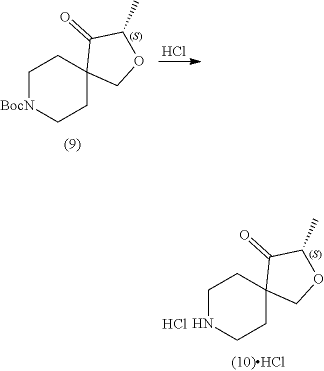

wherein R¹ and R² are each independently methyl or ethyl.

### Compound of Formula (11)

In another aspect, provided herein is a method of preparing a compound of Formula (11):

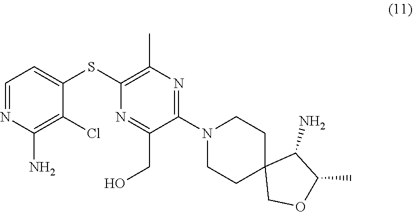 [SMILES: Cc1nc(N2CCC3(CC2)CO[C@@H](C)[C@H]3N)c(CO)nc1Sc1ccnc(N)c1Cl]

or a salt thereof, comprising converting the compound of Formula (10), or a salt thereof, to the compound of Formula (11) or a salt thereof. An overview of various intermediates and reaction steps for converting the compound of Formula (10), or a salt thereof, to the compound of Formula (11), or a salt thereof, is summarized above in Scheme 2, and a detailed description of the same is provided below.
   Compound of Formula (a-6)

In one aspect, provided herein is a method of preparing a compound of Formula (a-6):

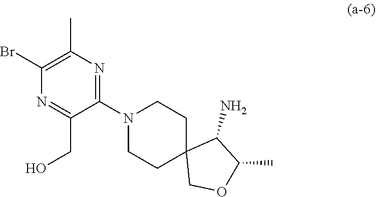 [SMILES: Cc1nc(N2CCC3(CC2)CO[C@@H](C)[C@H]3N)c(CO)nc1Br]

or a salt thereof, comprising:
reacting a compound of Formula (a-5):

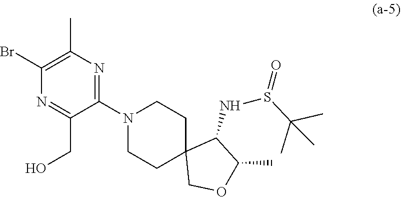 [SMILES: Cc1nc(N2CCC3(CC2)CO[C@@H](C)[C@H]3NS(=O)C(C)(C)C)c(CO)nc1Br]

with an acid to form the compound of Formula (a-6) or a salt thereof.

In some embodiments, the acid used for the reaction of the compound of Formula (a-5) is HCl, HBr, methanesulfonic acid, trifluoroacetic acid, or acetic acid. In some embodiments, the acid is HCl. In some embodiments, HCl is generated in situ by reaction of acetyl chloride, trimethylsilyl chloride, or AlCl₃ with an alcohol, such as methanol or ethanol.

In some embodiments, the reaction of the compound of Formula (a-5) with the acid is carried out using an alcohol as solvent. In some embodiments, the alcohol is ethanol, methanol, or isopropanol. In some embodiments, the alcohol is ethanol. In some embodiments, the alcohol is methanol. In some embodiments, the reaction is carried out using an ether, such as 1,4-dioxane, tetrahydrofuran, 2-methyltetrahydrofuran, or diethyl ether, as solvent. In some embodiments, the ether is tetrahydrofuran. In some embodiments, the reaction is carried out using a mixture of an ether and an alcohol, such as methanol or ethanol, as solvent. In some embodiments, the ether is 1,4-dioxane, tetrahydrofuran, 2-methyltetrahydrofuran, or diethyl ether. In some embodiments, the reaction is carried out using water as solvent. In some embodiments, the reaction is carried out using a mixture of water and an alcohol, such as methanol or ethanol. In some embodiments, the reaction is carried out using a biphasic solvent system. In some embodiments, the biphasic solvent system is a mixture of water and 2-methyltetrahydrofuran. In some embodiments, the reaction is carried out using an acidic biphasic solvent system, such as HCl in a mixture of water and 2-methyltetrahydrofuran. In some embodiments, the reaction is carried out using acidic ethyl acetate as solvent, such as HCl in ethyl acetate. In some embodiments, the reaction is carried out using acidic dioxane as solvent, such as HCl in dioxane.

In some embodiments, the reaction of the compound of Formula (a-5) with the acid is carried out at a temperature of about 0-25° C. In some embodiments, the reaction temperature is about 22° C. In some embodiments, the reaction is carried out at a temperature of about 0-100° C., such as about 35-90° C.

In some embodiments, the compound of Formula (a-5) is the compound of Formula (a-5a):

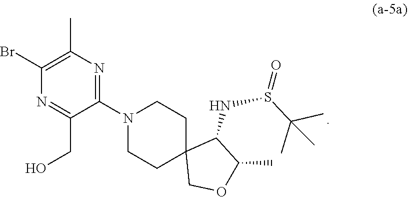 [SMILES: Cc1nc(N2CCC3(CC2)CO[C@@H](C)[C@H]3N[S@](=O)C(C)(C)C)c(CO)nc1Br]

Compound of Formula (a-5)

In a further aspect, provided herein is a method of preparing a compound of Formula (a-5):

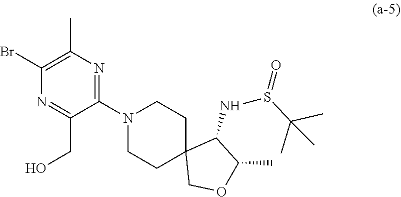 [SMILES: Cc1nc(N2CCC3(CC2)CO[C@@H](C)[C@H]3NS(=O)C(C)(C)C)c(CO)nc1Br]

comprising reacting a compound of Formula (a-2):

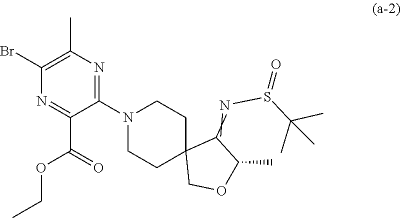 [SMILES: CCOC(=O)c1nc(Br)c(C)nc1N1CCC2(CC1)CO[C@@H](C)C2=NS(=O)C(C)(C)C]

with a reducing agent to form the compound of Formula (a-5).

In some embodiments, the reducing agent used for the reaction of the compound of Formula (a-2) is an organoaluminum hydride, an organoborane hydride, or a borohydride reagent. In some embodiments, the reducing agent is diisobutylaluminum hydride (DIBAL-H), LiBHEt₃, L-selectride, N-selectride, K-selectride, sodium borohydride, lithium borohydride, or potassium borohydride. In some embodiments, the reducing agent is diisobutylaluminum hydride (DIBAL-H). In some embodiments, DIBAL-H is used as a neat liquid. In some embodiments, DIBAL-H is used as an organic solution of tetrahydrofuran, toluene, cyclohexane, heptane or dichloromethane.

In some embodiments, the reaction of the compound of Formula (a-2) with the reducing agent is carried out using an aprotic solvent. In some embodiments, the aprotic solvent is tetrahydrofuran, 2-methyltetrahydrofuran, acetonitrile, 1,4-dioxane, toluene, dichloromethane, dichloroethane, chloroform, or a mixture thereof. In some embodiments, the aprotic solvent is 2-methyltetrahydrofuran.

In some embodiments, the reaction of the compound of Formula (a-2) with the reducing agent is carried out at a temperature of about −15 to −25° C. In some embodiments, the reaction temperature is about −20° C. In some embodiments, the reaction is carried out at a temperature of about −60 to 25° C. In some embodiments, the reaction temperature is about −35° C. In some embodiments, the reaction temperature is about −10° C. In some embodiments, the reaction temperature is about −35 to −10° C. In some embodiments, the reaction of the compound of Formula (a-2) with the reducing agent is carried out at a temperature of about −40 to 20° C. In some embodiments, the reaction of the compound of Formula (a-2) with the reducing agent is carried out at a temperature of about −30 to 30° C. In some embodiments, the reaction temperature is about −40° C. In some embodiments, the reaction temperature is about 0° C. In some embodiments, the reaction temperature is about 10° C. In some embodiments, the reaction temperature is about 20° C.

In some embodiments, the compound of Formula (a-2) is the compound of Formula (a-2a):

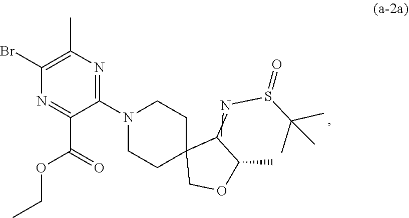 [SMILES: CCOC(=O)c1nc(Br)c(C)nc1N1CCC2(CC1)CO[C@@H](C)C2=NS(=O)C(C)(C)C]

and the compound of Formula (a-5) is the compound of Formula (a-5a):

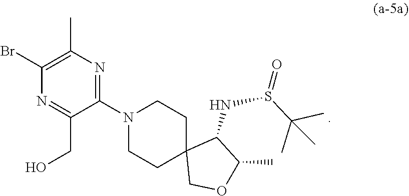 [SMILES: Cc1nc(N2CCC3(CC2)CO[C@@H](C)[C@H]3N[S@](=O)C(C)(C)C)c(CO)nc1Br]

Compound of Formula (a-2)

In a further aspect, provided herein is a method of preparing a compound of Formula (a-2):

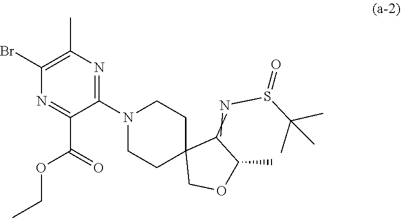 [SMILES: CCOC(=O)c1nc(Br)c(C)nc1N1CCC2(CC1)CO[C@@H](C)C2=NS(=O)C(C)(C)C]

comprising reacting the compound of Formula (a-1):

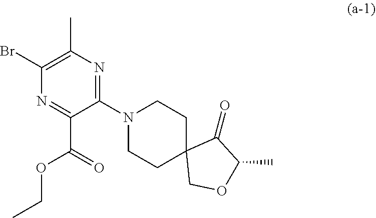 [SMILES: CCOC(=O)c1nc(Br)c(C)nc1N1CCC2(CC1)CO[C@@H](C)C2=O]

with the compound of Formula (A1):

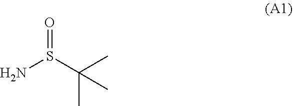

and a titanium alkoxide reagent to form the compound of Formula (a-2).

In some embodiments, the compound of Formula (a-2) has the structure of Formula (a-2a):

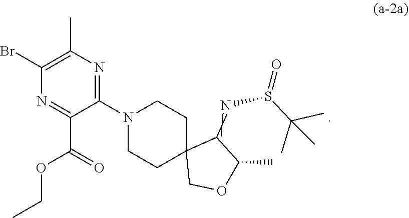 [SMILES: CCOC(=O)c1nc(Br)c(C)nc1N1CCC2(CC1)CO[C@@H](C)C2=N[S@](=O)C(C)(C)C]

In other embodiments, the compound of Formula (a-2) has the structure of Formula (a-2b):

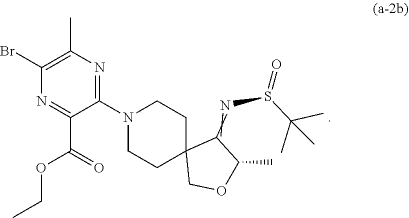 [SMILES: CCOC(=O)c1nc(Br)c(C)nc1N1CCC2(CC1)CO[C@@H](C)C2=N[S@@](=O)C(C)(C)C]

In the structures of Formula (a-2), Formula (a-2a), and Formula (a-2b), [symbol] represents a double bond having either E configuration or Z configuration. In some embodiments, [symbol] is a double bond having E configuration. In other embodiments, [symbol] is a double bond having Z configuration.

In some embodiments, the titanium alkoxide reagent used for the reaction of the compound of Formula (a-1) with the compound of Formula (A1) is Ti(OCH₂CH₃)₄. In some embodiments, the titanium alkoxide reagent is Ti(OCH(CH₃)₂)₄.

In some embodiments, the reaction of the compound of Formula (a-1) with the compound of Formula (A1) is carried out using an aprotic solvent. In some embodiments, the aprotic solvent is tetrahydrofuran, 2-methyltetrahydrofuran, methylcyclohexane, hexanes, cyclopentyl methyl ether, acetonitrile, 1,4-dioxane, toluene, dichloromethane, dichloroethane, or chloroform. In some embodiments, the aprotic solvent is 2-methyltetrahydrofuran.

In some embodiments, the reaction of the compound of Formula (a-1) with the compound of Formula (A1) is carried out at a temperature of about 70-90° C. In some embodiments, the reaction temperature is about 80° C. In some embodiments, the reaction of the compound of Formula (a-1) with the compound of Formula (A1) is carried out at the reflux temperature of the solvent. For example, the reaction of the compound of Formula (a-1) with the compound of Formula (A1) can be carried out at 100° C. using methylcyclohexane as solvent.

In some embodiments, the reaction of the compound of Formula (a-1) with the compound of Formula (A1) and the titanium alkoxide reagent provides the compound of Formula (a-2) as a mixture of the compounds of Formula (a-2a) and Formula (a-2b). In some embodiments, the mixture comprises about 50% or more of the compound of Formula (a-2a), and about 50% or less of the compound of Formula (a-2b). In some embodiments, the mixture comprises about 50-99% of the compound of Formula (a-2a), such as about 50%, 55%, 60%, 65%, 70%, 75%, 80%, 85%, 90%, 95%, or 99% of the compound of Formula (a-2a), and about 1-50% of the compound of Formula (a-2b), such as about 1%, 5%, 10%, 15%, 20%, 25%, 30%, 35%, 40%, 45%, or 50% of the compound of Formula (a-2b). In some embodiments, the mixture comprises about 80-99% of the compound of Formula (a-2a), such as about 80%, 81%, 82%, 83%, 84%, 85%, 86%, 87%, 88%, 89%, 90%, 91%, 92%, 93%, 94%, 95%, 96%, 97%, 98%, or 99% of the compound of Formula (a-2a), and about 1-20% of the compound of Formula (a-2b), such as about 1%, 2%, 3%, 4%, 5%, 6%, 7%, 8%, 9%, 10%, 11%, 12%, 13%, 14%, 15%, 16%, 17%, 18%, 19%, or 20% of the compound of Formula (a-2b). In some embodiments, the mixture comprises about 90% or more of the compound of Formula (a-2a). In some embodiments, the mixture comprises about 99% of the compound of Formula (a-2a). In some embodiments, the mixture comprises about 99% of the compound of Formula (a-2a), and about 1% of the compound of Formula (a-2b); about 98% of the compound of Formula (a-2a), and about 2% of the compound of Formula (a-2b); about 97% of the compound of Formula (a-2a), and about 3% of the compound of Formula (a-2b); about 96% of the compound of Formula (a-2a), and about 4% of the compound of Formula (a-2b); about 95% of the compound of Formula (a-2a), and about 5% of the compound of Formula (a-2b); about 94% of the compound of Formula (a-2a), and about 6% of the compound of Formula (a-2b); about 93% of the compound of Formula (a-2a), and about 7% of the compound of Formula (a-2b); about 92% of the compound of Formula (a-2a), and about 8% of the compound of Formula (a-2b); about 91% of the compound of Formula (a-2a), and about 9% of the compound of Formula (a-2b); or about 90% of the compound of Formula (a-2a), and about 10% of the compound of Formula (a-2b).

In some embodiments, the compound of Formula (A1) is the compound of Formula (A1a):

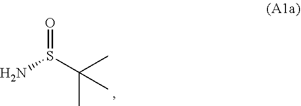

and the compound of Formula (a-2) is the compound of Formula (a-2a):

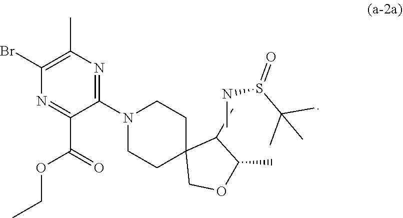

In other embodiments, the compound of Formula (A1) is the compound of Formula (A1b):

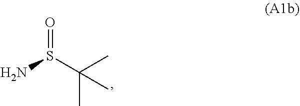

and the compound of Formula (a-2) is the compound of Formula (a-2b):

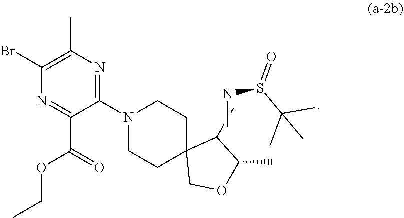

Compound of Formula (a-1)

In yet another aspect, provided herein is a method of preparing a compound of Formula (a-1):

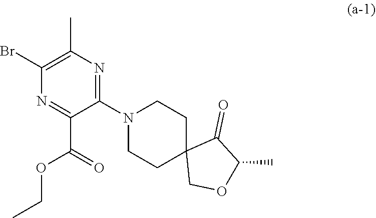 [SMILES: CCOC(=O)c1nc(Br)c(C)nc1N1CCC2(CC1)CO[C@@H](C)C2=O]

comprising reacting a compound of Formula (10):

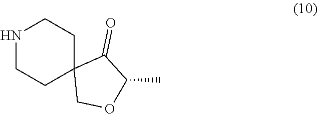

or a salt thereof,
with the compound of Formula (IV):

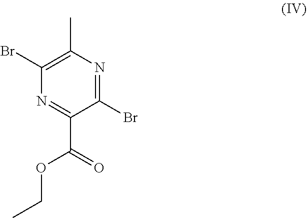

to form the compound of Formula (a-1).

In some embodiments, the compound of Formula (10) is provided as a salt. In some embodiments, the salt of the compound of Formula (10) is an HCl, HBr, trifluoroacetic acid, methanesulfonic acid, or H₂SO₄ salt. In some embodiments, the salt of the compound of Formula (10) is an HCl salt.

In some embodiments, the reaction of the compound of Formula (10) with the compound of Formula (IV) further comprises a base. In some embodiments, the base is K₂CO₃, Na₂CO₃, or NaHCO₃. In some embodiments, the base is K₂CO₃. In some embodiments, the base is a tertiary amine, such as a trialkylamine (for example, diisopropylethylamine or triethylamine). In some embodiments, the base is a trialkylamine.

In some embodiments, the reaction of the compound of Formula (10) with the compound of Formula (IV) is carried out using an aprotic solvent. In some embodiments, the aprotic solvent is tetrahydrofuran, 2-methyltetrahydrofuran, acetonitrile, 1,4-dioxane, toluene, dimethylsulfoxide, dimethylacetamide, N-methyl-2-pyrrolidone, dichloromethane, dichloroethane, or chloroform. In some embodiments, the aprotic solvent is dichloromethane.

In some embodiments, the reaction of the compound of Formula (10) with the compound of Formula (IV) is carried out at a temperature of about 30-50° C. In some embodiments, the reaction temperature is about 40° C. In some embodiments, the reaction is carried out at a temperature of about 10° C. to the reflux temperature of the solvent. In some embodiments, the reaction is carried out at the reflux temperature of the solvent. In some embodiments, the reaction is carried out at a temperature of about 30-180° C. In some embodiments, the reaction is carried out at a temperature of about 30-120° C. In some embodiments, the reaction is carried out at a temperature of about 30-150° C.

### Compound of Formula (IV)

In one aspect, provided herein is a method of preparing a compound of Formula (IV):

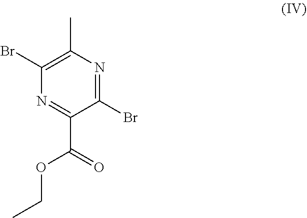

comprising reacting a compound of Formula (III):

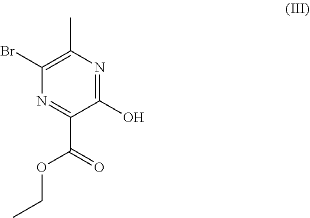

with POBr₃ to form the compound of Formula (IV).

In some embodiments, the reaction of the compound of Formula (III) with POBr₃ is carried out using an aprotic solvent. In some embodiments, the aprotic solvent is tetrahydrofuran, 2-methyltetrahydrofuran, acetonitrile, 1,4-dioxane, toluene, xylene, dichloromethane, dichloroethane, or chloroform. In some embodiments, the aprotic solvent is dichloromethane.

In some embodiments, the reaction of the compound of Formula (III) with POBr₃ is carried out at a temperature of about 70-90° C. In some embodiments, the reaction temperature is about 80° C. In some embodiments, the reaction of the compound of Formula (III) with POBr₃ is carried out at a temperature of about 30-40° C. In some embodiments, the reaction of the compound of Formula (III) with POBr₃ is carried out at the reflux temperature of solvent. In some embodiments, the reaction temperature is about 110 or 120° C. In some embodiments, the reaction temperature is about 30 or 40° C.

In some embodiments, the reaction of the compound of Formula (III) with POBr₃ further comprises dimethylformamide as a catalyst.

Also provided herein is a method of preparing the compound of Formula (IV) comprising reacting the compound of Formula (III) with N-bromo-succinimide and triphenylphosphine to form the compound of Formula (IV). In some embodiments, the reaction of the compound of Formula (III) with N-bromo-succinimide and triphenylphosphine is carried out using an ether, such as 1,4-dioxane, as solvent. In some embodiments, the reaction of the compound of Formula (III) with N-bromo-succinimide and triphenylphosphine is carried out at a temperature of about 25-100° C. In some embodiments, the reaction is carried out at a temperature of about 100° C. In some embodiments, the reaction is carried out at the reflux temperature of the solvent.

### Compound of Formula (VI)

In still a further aspect, provided herein is a method of preparing a compound of Formula (VI):

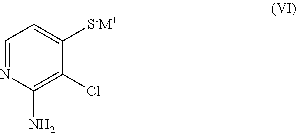

wherein M⁺ is Lit, Na⁺, or K⁺,
comprising reacting a compound of Formula (V):

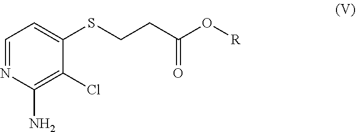

wherein R is C₁-C₁₂ alkyl,
with a base to form the compound of Formula (VI).

In some embodiments, R is C₁-C₁₂ alkyl. In some embodiments, Ris C₁-C₁₀ alkyl. In some embodiments, R is C₁-C₆ alkyl. In some embodiments, R is C₁-C₃ alkyl. In some embodiments, R is methyl, ethyl, or propyl. In some embodiments, R is ethyl. In some embodiments, R is C₁-C₈ alkyl. In some embodiments, R is 1-ethylhexyl, 2-ethylhexyl, 3-ethylhexyl, 4-ethylhexyl, or 5-ethylhexyl. In some embodiments, R is 2-ethylhexyl and the compound of Formula (V) is the compound of Formula (Va):

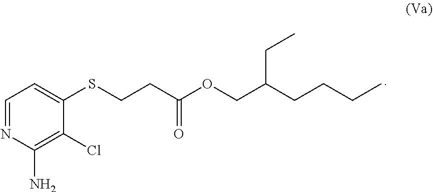 [SMILES: CCCCC(CC)COC(=O)CCSc1ccnc(N)c1Cl]

In some embodiments, the base used for the reaction of the compound of Formula (V) is NaOH, KOH, LiOH, KOCH₃, NaOCH₃, LiOCH₃, KOCH₂CH₃, NaOCH₂CH₃, LiOCH₂CH₃, KO(tert-butyl), NaO(tert-butyl), or LiO(tert-butyl). In some embodiments, the base is KOCH₂CH₃.

In some embodiments, the reaction of the compound of Formula (V) with the base is carried out using an aprotic solvent. In some embodiments, the aprotic solvent is tetrahydrofuran, 2-methyltetrahydrofuran, acetonitrile, 1,4-dioxane, toluene, methyl tert-butyl ether, or xylene. In some embodiments, the aprotic solvent is 2-methyltetrahydrofuran. In some embodiments, the reaction of the compound of Formula (V) with the base is carried out using a mixture of an alcohol and an aprotic solvent. In some embodiments, the alcohol is ethanol or methanol. In some embodiments, the reaction of the compound of Formula (V) with the base is carried out using a mixture of 2-methyltetrahydrofuran and an alcohol, such as methanol, ethanol, or isopropanol. In some embodiments, the alcohol is ethanol. In some embodiments, the reaction of the compound of Formula (V) with the base is carried out using a protic solvent such as an alcohol, for example, methanol or ethanol.

In some embodiments, the reaction of the compound of Formula (V) with the base is carried out at a temperature of about 15-25° C. In some embodiments, the reaction temperature is about 22° C. In some embodiments, the reaction of the compound of Formula (V) with the base is carried out at a temperature of about 0-50° C. In some embodiments, the reaction of the compound of Formula (V) with the base is carried out at a temperature of about 10-30° C. In some embodiments, the reaction of the compound of Formula (V) with the base is carried out at the reflux temperature of the solvent. In some embodiments, the reaction temperature is about 10-120° C.

Compound of Formula (a-7)

Also provided herein is a method of preparing a compound of Formula (a-7):

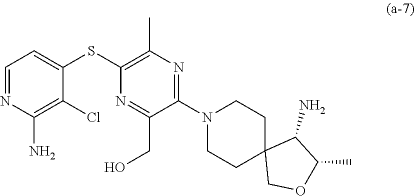 [SMILES: Cc1nc(N2CCC3(CC2)CO[C@@H](C)[C@H]3N)c(CO)nc1Sc1ccnc(N)c1Cl]

or a salt thereof,
comprising reacting the compound of Formula (a-6)

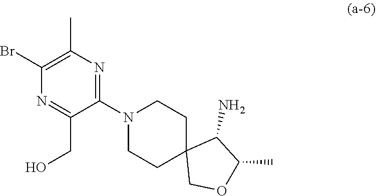 [SMILES: Cc1nc(N2CCC3(CC2)CO[C@@H](C)[C@H]3N)c(CO)nc1Br]

or a salt thereof, with a compound of Formula (VI):

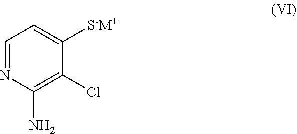

wherein M⁺ is Lit, Na⁺, or K⁺,
to form the compound of Formula (a-7) or a salt thereof.

In some embodiments, M⁺ is Lit. In some embodiments, M⁺ is Nat. In some embodiments, M⁺ is K⁺ and the compound of Formula (VI) is a compound of Formula (VIa):

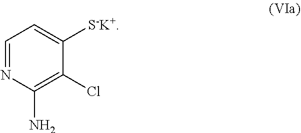

In some embodiments, the reaction of the compound of Formula (a-6), or a salt thereof, with the compound of Formula (VI) further comprises a copper salt. In some embodiments, the copper salt is a copper (I) salt. In other embodiments, the copper salt is a copper (II) salt. In some embodiments, the copper salt is CuI. In some embodiments, the copper salt is CuBr. In some embodiments, the copper salt is copper acetate hydrate (i.e., Cu(CO₂CH₃)₂·xH₂O). In some embodiments, the copper salt comprises imidazole. In other embodiments, the reaction of the compound of Formula (a-6), or a salt thereof, with the compound of Formula (VI) is carried out in the absence of a copper salt.

In some embodiments, the reaction of the compound of Formula (a-6), or a salt thereof, with the compound of Formula (VI) is carried out using an aprotic solvent. In some embodiments, the aprotic solvent is tetrahydrofuran, 2-methyltetrahydrofuran, acetonitrile, butyronitrile, sulfolane, dimethylformamide, N-methyl-2-pyrrolidone, dimethylacetamide, morpholine, 1,4-dioxane, ethylene glycol, toluene, pyridine, dichloromethane, dichloroethane, or chloroform. In some embodiments, the aprotic solvent is pyridine. In some embodiments, the aprotic solvent is an alkylene glycol, such as methylene glycol, ethylene glycol, or propylene glycol. In some embodiments, the reaction of the compound of Formula (a-6), or a salt thereof, with the compound of Formula (VI) is carried out using a mixture of an aprotic solvent and an alcohol, such as methanol, ethanol, or isopropanol. In some embodiments, the reaction of the compound of Formula (a-6), or a salt thereof, with the compound of Formula (VI) is carried out using a mixture of an alkylene glycol, such as methylene glycol, ethylene glycol, or propylene glycol, and an alcohol, such as methanol, ethanol, or isopropanol. In some embodiments, the reaction of the compound of Formula (a-6), or a salt thereof, with the compound of Formula (VI) is carried out using a mixture of ethylene glycol and isopropanol.

In some embodiments, the reaction of the compound of Formula (a-6), or a salt thereof, with the compound of Formula (VI) is carried out at a temperature of about 50-130° C. In some embodiments, the reaction temperature is about 80° C. In some embodiments, the reaction of the compound of Formula (a-6), or a salt thereof, with the compound of Formula (VI) is carried out at a temperature of about 100-130° C. In some embodiments, the reaction temperature is about 115° C. In some embodiments, the reaction of the compound of Formula (a-6), or a salt thereof, with the compound of Formula (VI) is carried out at reflux temperature of the solvent.

In some embodiments, the reaction of the compound of Formula (a-6), or a salt thereof, with the compound of Formula (VI) is carried out at an elevated pressure. In some embodiments, the pressure of the reaction is from about 1-10 bar (about 14.5-145 psi).

In some embodiments, the reaction of the compound of Formula (a-6), or a salt thereof, with the compound of Formula (VI) is carried out using flow chemistry. In some embodiments, the flow chemistry is performed at an elevated pressure, such as from about 1-10 bar (about 14.5-145 psi).

In some embodiments, provided herein is a method for preparing the compound of Formula (11), or a salt thereof, according to Scheme 4.

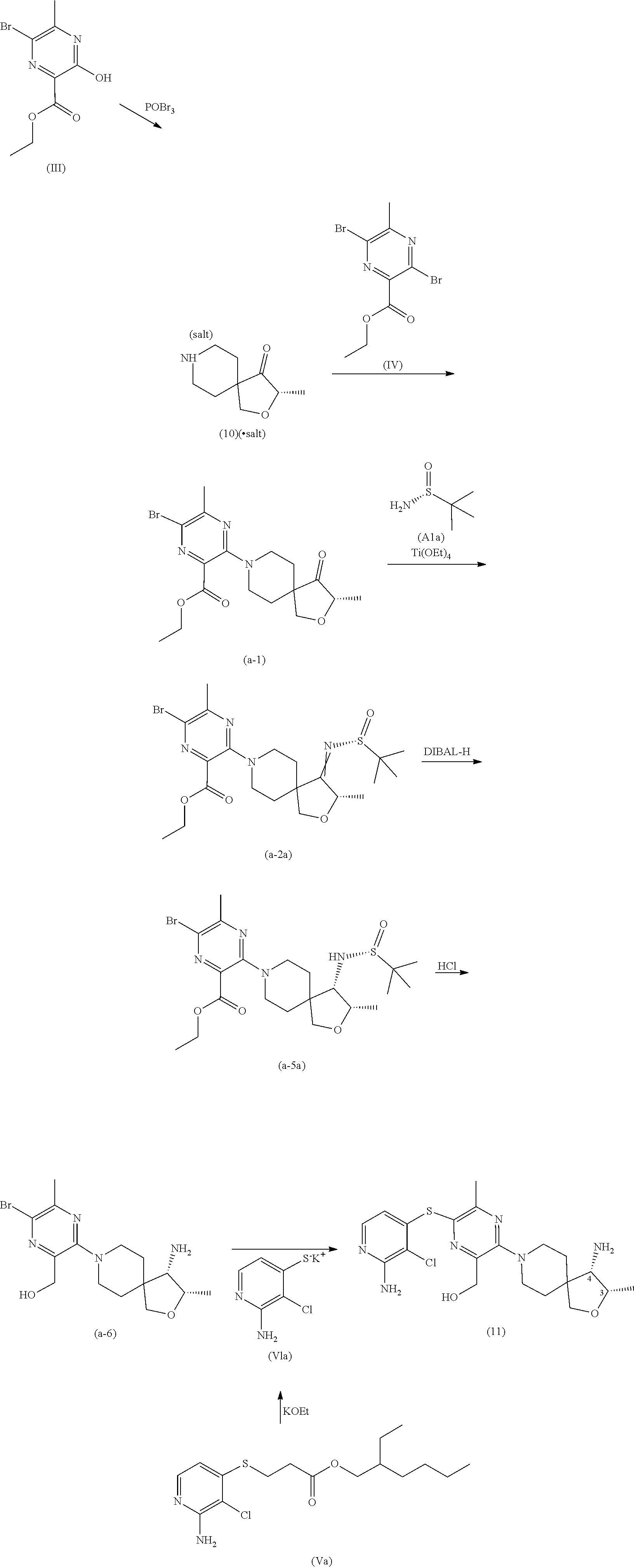

In some embodiments of the synthetic methods disclosed herein, the absolute configuration at the C₄ position on the spiro-cycle of the compound of Formula (11), the numbering of which is shown in Scheme 4, is promoted by the absolute stereochemistry at the sulfur atom of the compound of Formula (A1a). In some embodiments, the(S) absolute configuration at the C₄ position on the spiro-cycle of the compound of Formula (11) is promoted by the (R) absolute stereochemistry at the sulfur atom of the compound of Formula (A1a) and is carried forward in the compounds of Formula (a-2a), (a-5a), and (a-6).

In other embodiments, the preparation of the compound of Formula (11), or a salt thereof, comprises the reactions shown in Scheme 5.

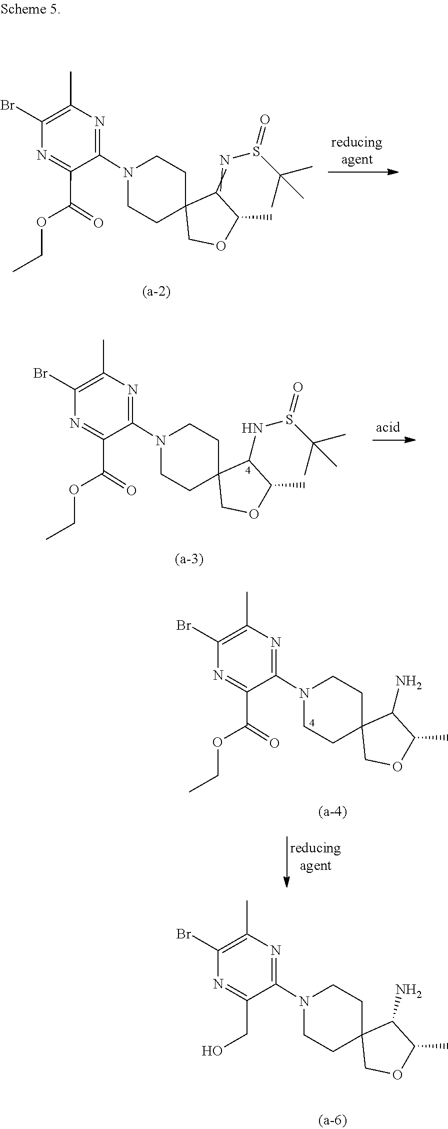

The sulfinyl imine compound of Formula (a-2) can be reduced using, for example DIBAL-H, to the sulfinamide compound of Formula (a-3), followed by treatment with an acid to form the amine compound of Formula (a-4). The ester moiety of the compound of Formula (a-4) can then be reduced using, for example DIBAL-H, to form the compound of Formula (a-6), and subsequently used to prepare the compound of Formula (11) as outlined in Schemes 2 and 4. In some embodiments, the compound of Formula (a-3) has(S) absolute configuration at C₄, and the compound of Formula (a-4) has(S) absolute configuration at C₄. In some embodiments, the compound of Formula (a-3) has the structure of Formula (a-3a), and the compound of Formula (a-4) has the structure of Formula (a-4a). Accordingly, in some embodiments, the preparation of the compound of Formula (11), or a salt thereof, comprises the reactions shown in Scheme 6.

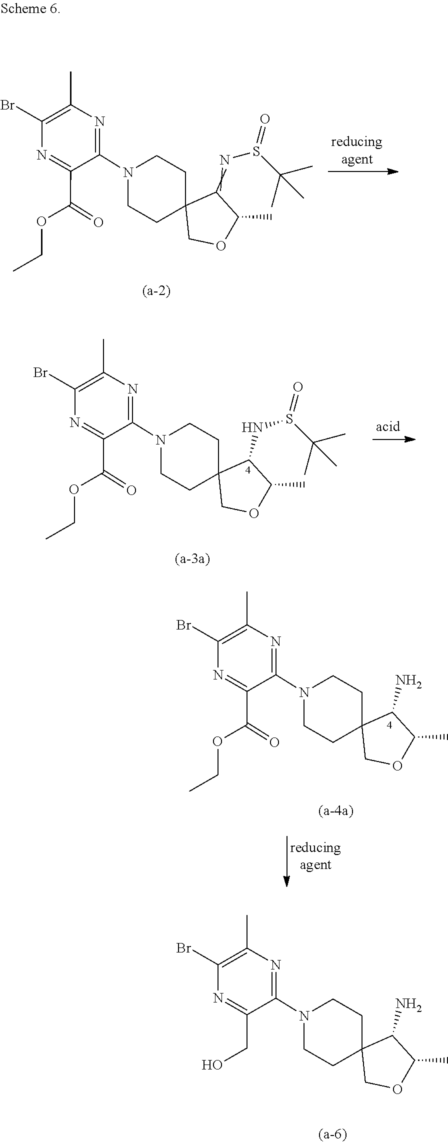

Compound of Formula (a-3)

Also provided herein is a method of preparing the compound of Formula (a-3)

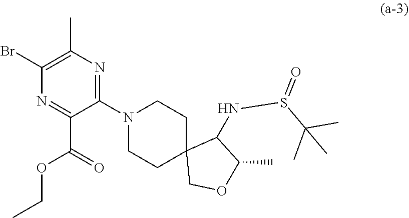 [SMILES: CCOC(=O)c1nc(Br)c(C)nc1N1CCC2(CC1)CO[C@@H](C)C2NS(=O)C(C)(C)C]

comprising reacting a compound of Formula (a-2):

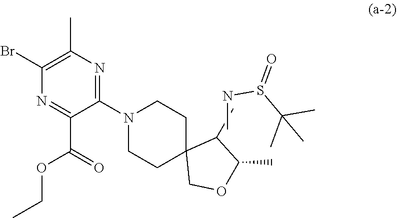

with a reducing agent to form the compound of Formula (a-3).

In some embodiments, the compound of Formula (a-3) has the structure of Formula (a-3a):

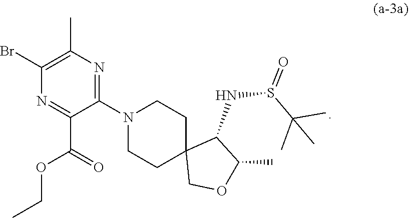 [SMILES: CCOC(=O)c1nc(Br)c(C)nc1N1CCC2(CC1)CO[C@@H](C)[C@H]2N[S@](=O)C(C)(C)C]

In other embodiments, the compound of Formula (a-3) has the structure of Formula (a-3b):

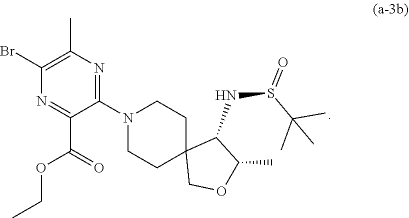 [SMILES: CCOC(=O)c1nc(Br)c(C)nc1N1CCC2(CC1)CO[C@@H](C)[C@H]2N[S@@](=O)C(C)(C)C]

In some embodiments, the reducing agent used for the reaction of the compound of Formula (a-2) is an organoaluminum hydride, an organoborane hydride, or a borohydride reagent. In some embodiments, the reducing agent is diisobutylaluminum hydride (DIBAL-H), LiBHEt₃, L-selectride, N-selectride, K-selectride, sodium borohydride, lithium borohydride, or potassium borohydride. In some embodiments, the reducing agent is diisobutylaluminum hydride (DIBAL-H). In some embodiments, DIBAL-H is used as a neat liquid. In some embodiments, DIBAL-H is used as an organic solution of tetrahydrofuran, toluene, cyclohexane, heptane, or dichloromethane.

In some embodiments, the reaction of the compound of Formula (a-2) with the reducing agent is carried out using an aprotic solvent. In some embodiments, the aprotic solvent is tetrahydrofuran, 2-methyltetrahydrofuran, acetonitrile, 1,4-dioxane, toluene, dichloromethane, dichloroethane, chloroform, or a mixture thereof. In some embodiments, the aprotic solvent is 2-methyltetrahydrofuran or toluene. In some embodiments, the aprotic solvent is 2-methyltetrahydrofuran. In some embodiments, the aprotic solvent is toluene.

In some embodiments, the reaction of the compound of Formula (a-2) with the reducing agent provides the compound of Formula (a-3) as a mixture of the compounds of Formula (a-3a) and Formula (a-3b). In some embodiments, the mixture comprises about 50% or more of the compound of Formula (a-3a), and about 50% or less of the compound of Formula (a-3b). In some embodiments, the mixture comprises about 50-99% of the compound of Formula (a-3a), such as about 50%, 55%, 60%, 65%, 70%, 75%, 80%, 85%, 90%, 95%, or 99% of the compound of Formula (a-3a), and about 1-50% of the compound of Formula (a-3b), such as about 1%, 5%, 10%, 15%, 20%, 25%, 30%, 35%, 40%, 45%, or 50% of the compound of Formula (a-3b). In some embodiments, the mixture comprises about 80-99% of the compound of Formula (a-3a), such as about 80%, 81%, 82%, 83%, 84%, 85%, 86%, 87%, 88%, 89%, 90%, 91%, 92%, 93%, 94%, 95%, 96%, 97%, 98%, or 99% of the compound of Formula (a-3a), and about 1-20% of the compound of Formula (a-3b), such as about 1%, 2%, 3%, 4%, 5%, 6%, 7%, 8%, 9%, 10%, 11%, 12%, 13%, 14%, 15%, 16%, 17%, 18%, 19%, or 20% of the compound of Formula (a-3b). In some embodiments, the mixture comprises about 90% or more of the compound of Formula (a-3a). In some embodiments, the mixture comprises about 99% of the compound of Formula (a-3a), and about 1% of the compound of Formula (a-3b); about 98% of the compound of Formula (a-3a), and about 2% of the compound of Formula (a-3b); about 97% of the compound of Formula (a-3a), and about 3% of the compound of Formula (a-3b); about 96% of the compound of Formula (a-3a), and about 4% of the compound of Formula (a-3b); about 95% of the compound of Formula (a-3a), and about 5% of the compound of Formula (a-3b); about 94% of the compound of Formula (a-3a), and about 6% of the compound of Formula (a-3b); about 93% of the compound of Formula (a-3a), and about 7% of the compound of Formula (a-3b); about 92% of the compound of Formula (a-3a), and about 8% of the compound of Formula (a-3b); about 91% of the compound of Formula (a-3a), and about 9% of the compound of Formula (a-3b); or about 90% of the compound of Formula (a-3a), and about 10% of the compound of Formula (a-3b).

Compound of Formula (a-4)

Also provided herein is a method of preparing a compound of Formula (a-4):

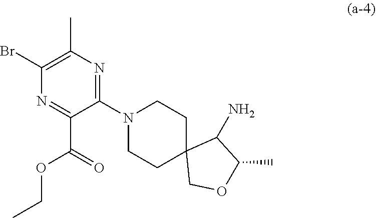 [SMILES: CCOC(=O)c1nc(Br)c(C)nc1N1CCC2(CC1)CO[C@@H](C)C2N]

or a salt thereof, comprising:
reacting a compound of Formula (a-3):

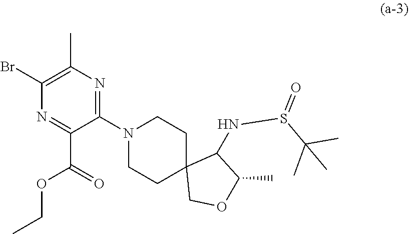 [SMILES: CCOC(=O)c1nc(Br)c(C)nc1N1CCC2(CC1)CO[C@@H](C)C2NS(=O)C(C)(C)C]

with an acid to form the compound of Formula (a-4) or a salt thereof.

In some embodiments, the compound of Formula (a-4) is a salt. In some embodiments, the salt of the compound of Formula (a-4) is an HCl, a methanesulfonic acid, or an HBr salt. In some embodiments, the salt of the compound of Formula (a-4) is an HCl salt. In some embodiments, the salt of the compound of Formula (a-4) is a methanesulfonic acid salt. In some embodiments, the salt of the compound of Formula (a-4) is an HBr salt.

In some embodiments, the acid used in the reaction of the compound of Formula (a-3) is HCl, HBr, methanesulfonic acid, trifluoroacetic acid, or acetic acid. In some embodiments, the acid is HCl. In some embodiments, HCl is generated in situ by reaction of acetyl chloride, trimethylsilyl chloride, or AlCl₃ with an alcohol, such as methanol or ethanol.

In some embodiments, the reaction of the compound of Formula (a-3) with the acid is carried out using an alcohol as solvent. In some embodiments, the alcohol is ethanol, methanol, or isopropanol. In some embodiments, the alcohol is ethanol. In some embodiments, the alcohol is methanol. In some embodiments, the reaction is carried out using a mixture of an ether and an alcohol, such as methanol or ethanol, as solvent. In some embodiments, the ether is 1,4-dioxane, tetrahydrofuran, 2-methyltetrahydrofuran, or diethyl ether. In some embodiments, the reaction is carried out using water as solvent. In some embodiments, the reaction is carried out using a mixture of water and alcohol, such as methanol or ethanol. In some embodiments, the reaction is carried out using a biphasic solvent system. In some embodiments, the biphasic solvent system is a mixture of water and 2-methyltetrahydrofuran. In some embodiments, the reaction is carried out using an acidic biphasic solvent system, such as HCl in a mixture of water and 2-methyltetrahydrofuran. In some embodiments, the reaction is carried out using acidic ethyl acetate as solvent, such as HCl in ethyl acetate. In some embodiments, the reaction is carried out using acidic dioxane as solvent, such as HCl in dioxane.

In some embodiments, the reaction of the compound of Formula (a-3) with the acid is carried out at a temperature of about 0-25° C. In some embodiments, the reaction temperature is about 22° C. In some embodiments, the reaction is carried out at a temperature of about 0-100° C., such as about 35-90° C.

In some embodiments, the compound of Formula (a-3) has the structure of Formula (3a):

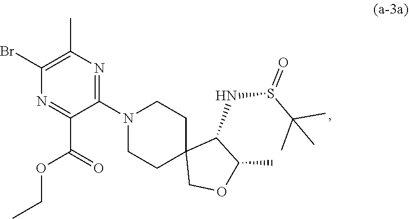 [SMILES: CCOC(=O)c1nc(Br)c(C)nc1N1CCC2(CC1)CO[C@@H](C)[C@H]2N[S@](=O)C(C)(C)C]

and the compound of Formula (a-4) has the structure of Formula (a-4a):

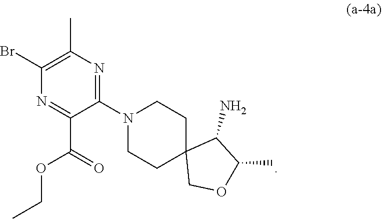 [SMILES: CCOC(=O)c1nc(Br)c(C)nc1N1CCC2(CC1)CO[C@@H](C)[C@H]2N]

In some embodiments, reaction of the compound of Formula (a-3) with the acid to provide the compound of Formula (a-4), or a salt thereof, comprises a mixture of the compounds of Formula (a-3a) and Formula (a-3b). In some embodiments, the mixture comprises about 50% or more of the compound of Formula (a-3a), and about 50% or less of the compound of Formula (a-3b). In some embodiments, the mixture comprises about 50-99% of the compound of Formula (a-3a), such as about 50%, 55%, 60%, 65%, 70%, 75%, 80%, 85%, 90%, 95%, or 99% of the compound of Formula (a-3a), and about 1-50% of the compound of Formula (a-3b), such as about 1%, 5%, 10%, 15%, 20%, 25%, 30%, 35%, 40%, 45%, or 50% of the compound of Formula (a-3b). In some embodiments, the mixture comprises about 80-99% of the compound of Formula (a-3a), such as about 80%, 81%, 82%, 83%, 84%, 85%, 86%, 87%, 88%, 89%, 90%, 91%, 92%, 93%, 94%, 95%, 96%, 97%, 98%, or 99% of the compound of Formula (a-3a), and about 1-20% of the compound of Formula (a-3b), such as about 1%, 2%, 3%, 4%, 5%, 6%, 7%, 8%, 9%, 10%, 11%, 12%, 13%, 14%, 15%, 16%, 17%, 18%, 19%, or 20% of the compound of Formula (a-3b). In some embodiments, the mixture comprises at least about 80% of the compound of Formula (a-3a), and no more than 20% of the compound of Formula (a-3b). In some embodiments, the mixture comprises about 80% or more of the compound of Formula (a-3a), and 20% or less of the compound of Formula (a-3b). In some embodiments, the mixture comprises about 90% or more of the compound of Formula (a-3a). In some embodiments, the mixture comprises about 99% of the compound of Formula (a-3a). In some embodiments, the mixture comprises about 99% of the compound of Formula (a-3a), and about 1% of the compound of Formula (a-3b); about 98% of the compound of Formula (a-3a), and about 2% of the compound of Formula (a-3b); about 97% of the compound of Formula (a-3a), and about 3% of the compound of Formula (a-3b); about 96% of the compound of Formula (a-3a), and about 4% of the compound of Formula (a-3b); about 95% of the compound of Formula (a-3a), and about 5% of the compound of Formula (a-3b); about 94% of the compound of Formula (a-3a), and about 6% of the compound of Formula (a-3b); about 93% of the compound of Formula (a-3a), and about 7% of the compound of Formula (a-3b); about 92% of the compound of Formula (a-3a), and about 8% of the compound of Formula (a-3b); about 91% of the compound of Formula (a-3a), and about 9% of the compound of Formula (a-3b); or about 90% of the compound of Formula (a-3a), and about 10% of the compound of Formula (a-3b).

Although the synthetic reactions disclosed herein have been described using bromo as the leaving group for compounds of Formula (a-1), (a-2), (a-3), (a-4), (a-5), (a-6), (III), (IV), and variations thereof, it is understood that other leaving groups may be used to achieve similar chemical transformations. Suitable leaving groups that can be used include, without limitation, other halogens such as chloro or iodo, sulfonate esters such as tosylate or mesylate, and perfluoroalkylsulfonates such as triflate.

Accordingly, encompassed herein is the use of the compounds of Formula (a-1-A), (a-2-A), (a-3-A), (a-4-A), (a-5-A), (a-6-A), (III-A), and (IV-A) in the synthesis of Compound (11) or a salt thereof:

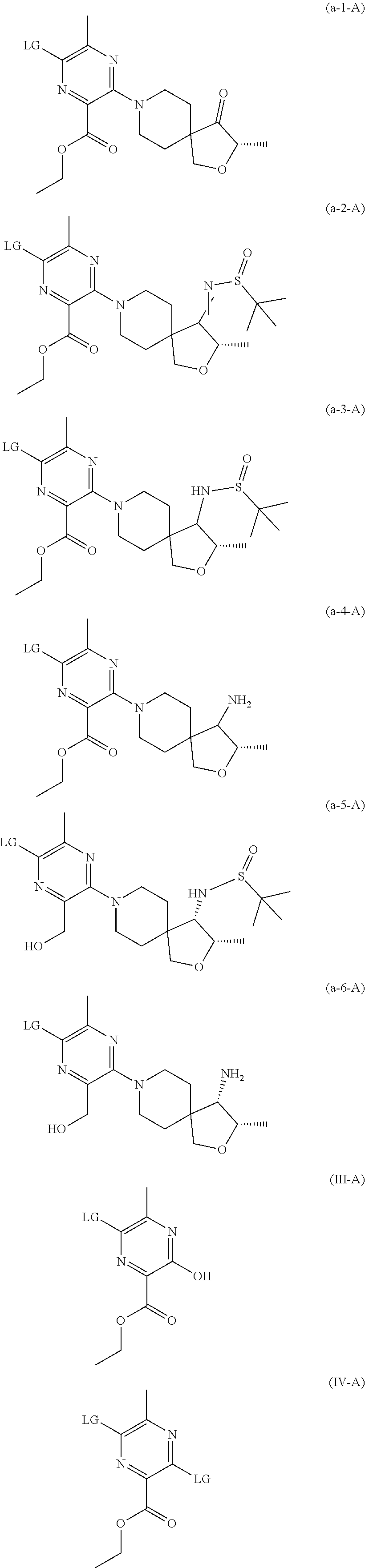

wherein LG is a suitable leaving group as described herein.

Similarly, although the synthetic reactions disclosed herein have been described using tert-butyl attached to the sulfur atom for compounds of Formula (a-2), (a-3), (a-5), (A1), and variations thereof, it is understood that other groups (R′) may be used to achieve similar chemical transformations. Suitable R′ groups that can be used include, without limitation, other alkyl groups such as 2-methylbutyl, and aryl groups such as p-tolyl.

Accordingly, encompassed herein is the use of the compounds of Formula (a-2-B), (a-3-B) (a-5-B), and (A1-B) in the synthesis of Compound (11) or a salt thereof:

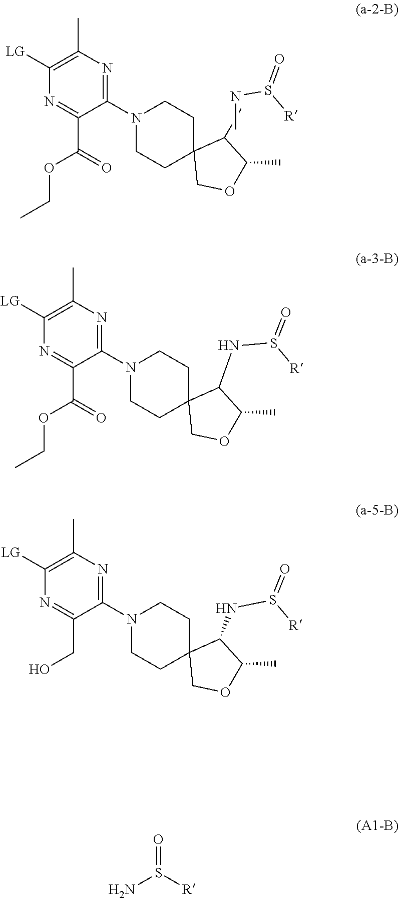

wherein LG is a suitable leaving group as described herein, and R′ is a suitable moiety as described herein.

In addition, although the synthetic reactions disclosed herein have been described using Boc (tert-butyloxocarbonyl) as the protecting group for the compound of Formula (9), and variations thereof, it is understood that other protecting groups may be used to achieve similar chemical transformations. Suitable protecting groups that can be used include, without limitation, benzyl and benzyloxycarbonyl. In some embodiments, an acid-labile protecting group is used.

Accordingly, encompassed herein is the use of the compounds of Formula (9-C) in the synthesis of Compound (11) or a salt thereof:

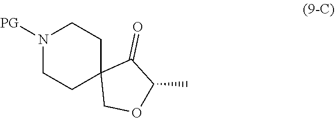

wherein PG is a suitable protecting group as described herein.

It will be understood that any of the compounds disclosed herein which exist in free base or acid form can be converted to their salts by treatment with the appropriate inorganic or organic base or acid by methods known to one skilled in the art. Similarly, salts of the compounds of the disclosure can be converted to their free base or acid form by standard techniques. Accordingly, where appropriate, salts of the compounds disclosed herein can be used in the synthetic methods in place of a described free base or acid form. Conversely, where appropriate, free base or acid forms of the compounds disclosed herein can be used in the synthetic methods in place of a described salt form.

### Compositions and Methods of Use

Compositions and uses of the compound of Formula (11), or a salt thereof, in treating or preventing a disease associated with SHP2 modulation in a subject in need thereof is described in U.S. Pat. No. 10,590,090, the disclosure of which is incorporated herein by reference.

Accordingly, in one aspect, provided herein is a method of treating a disease associated with SHP2 modulation in a subject in need thereof, comprising administering a therapeutically effective amount of the compound of Formula (11), or a salt thereof, prepared according to any of the methods disclosed herein, to the subject. In some embodiments, provided herein is a method of preventing a disease associated with SHP2 modulation in a subject in need thereof, comprising administering a therapeutically effective amount of the compound of Formula (11), or a salt thereof, prepared according to any of the methods disclosed herein, to the subject.

In some embodiments, provided herein is the use of the compound of Formula (11), or a salt thereof, prepared according to any of the methods disclosed herein, in the manufacture of a medicament for treating or preventing a disease associated with SHP2 modulation.

In some embodiments, provided herein is the use of the compound of Formula (11), or a salt thereof, prepared according to any of the methods disclosed herein, for treating or preventing a disease associated with SHP2 modulation in a subject in need thereof. In some embodiments, provided herein is the compound of Formula (11), or a salt thereof, prepared according to any of the methods disclosed herein, for treating or preventing a disease associated with SHP2 modulation in a subject in need thereof.

Non-limiting examples of a disease associated with SHP2 modulation include Noonan Syndrome, Leopard Syndrome, juvenile myelomonocytic leukemias, neuroblastoma, melanoma, acute myeloid leukemia, breast cancer, lung cancer, and colon cancer.

### Exemplary Embodiments

The present disclosure is further described by the following embodiments. The features of each of the embodiments are combinable with any of the other embodiments where appropriate and practical.

Embodiment P1. A method of preparing a compound of Formula (7):

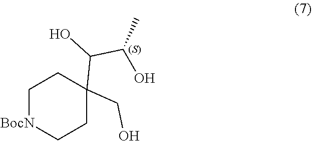

comprising:
reacting a compound of Formula (6):

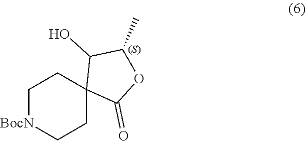

with a reducing agent to form the compound of Formula (7).

Embodiment P2. The method of embodiment P1, wherein the reducing agent is an organoaluminum hydride, an aluminum hydride, an organoborane hydride, or a borohydride reagent.

Embodiment P3. The method of embodiment P1 or P2, wherein the reducing agent is diisobutylaluminum hydride (DIBAL-H), sodium bis(2-methoxyethoxy)aluminium hydride (Red-Al), LiAlH₄, LiBHEt₃, L-selectride, N-selectride, K-selectride, sodium borohydride, lithium borohydride, calcium borohydride, or potassium borohydride.

Embodiment P4. The method of embodiment P3, wherein the reducing agent is lithium borohydride.

Embodiment P5. The method of any one of embodiments P1-P4, wherein the reaction is carried out using an alcohol, an aprotic solvent, or a mixture thereof as solvent.

Embodiment P6. The method of embodiment P5, wherein the reaction is carried out using a mixture of an alcohol and an aprotic solvent.

Embodiment P7. The method of embodiment P5 or P6, wherein the alcohol is ethanol, methanol, or isopropanol.

Embodiment P8. The method of any one of embodiments P5-P7, wherein the aprotic solvent is tetrahydrofuran, 2-methyltetrahydrofuran, acetonitrile, methyl tert-butyl ether (MBTE), cyclopentylmethylether, or 1,4-dioxane.

Embodiment P9. The method of any one of embodiments P5-P8, wherein the reaction is carried out using a mixture of methanol and tetrahydrofuran.

Embodiment P10. The method of any one of embodiments P5-P8, wherein the reaction is carried out using a mixture of ethanol and tetrahydrofuran.

Embodiment P11. The method of any one of embodiments P1-P10, wherein the reaction is carried out at a temperature of about 0 to 30° C.

Embodiment P12. The method of embodiment P11, wherein the reaction is carried out at a temperature of about 10 to 20° C.

Embodiment P13. A method of preparing a compound of Formula (7):

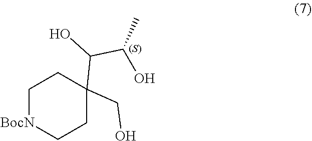

comprising reacting a compound of Formula (5):

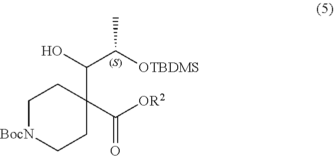

wherein R² is C₁-C₆ alkyl, and TBDMS is tert-butyldimethylsilyl ether,
with a deprotecting agent.

Embodiment P14. A method of preparing a compound of Formula (6):

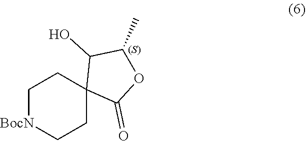

comprising reacting a compound of Formula (5):

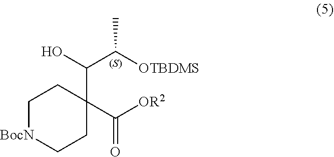

wherein R² is C₁-C₆ alkyl, and TBDMS is tert-butyldimethylsilyl ether,
with a deprotecting agent to form the compound of Formula (6).

Embodiment P15. The method of any one of embodiments P1-P12, wherein the compound of Formula (6) is prepared by reacting a compound of Formula (5):

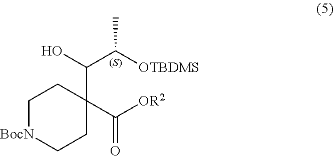

wherein R² is C₁-C₆ alkyl, and TBDMS is tert-butyldimethylsilyl ether,
with a deprotecting agent to form the compound of Formula (6).

Embodiment P16. The method of any one of embodiments P13-P15, wherein R² is methyl or ethyl.

Embodiment P17. The method of any one of embodiments P13-P16, wherein the deprotecting agent is a source of fluoride ion, acetyl chloride, N-iodosuccinimide, HCl, acetic acid, formic acid, phosphoric acid, FeCl₃, AlCl₃, CeCl₃, oxalyl chloride, isobutyl chloroformate, ethyl chloroformate, or thionyl chloride.

Embodiment P18. The method of embodiment P17, wherein the deprotecting agent is a source of fluoride ion.

Embodiment P19. The method of embodiment P18, wherein the source of fluoride ion is tetra-n-butylammonium fluoride (TBAF), NH₄F, CsF, HF⋅pyridine, or HF·Et₃N.

Embodiment P20. The method of embodiment P19, wherein the source of fluoride ion is tetra-n-butylammonium fluoride (TBAF).

Embodiment P21. The method of any one of embodiments P13-P20, wherein the reaction of the compound of Formula (5) with the deprotecting agent is carried out using an aprotic solvent.

Embodiment P22. The method of embodiment P21, wherein the aprotic solvent used for the reaction of the compound of Formula (5) with the deprotecting agent is tetrahydrofuran, 2-methyltetrahydrofuran, acetonitrile, 1,4-dioxane, toluene, dichloromethane, dichloroethane, chloroform, methyl tert-butyl ether (MBTE), cyclopentylmethylether, or a mixture thereof.

Embodiment P23. The method of embodiment P22, wherein the aprotic solvent used for the reaction of the compound of Formula (5) with the deprotecting agent is tetrahydrofuran.

Embodiment P24. The method of any one of embodiments P13-P23, wherein the reaction of the compound of Formula (5) with the deprotecting agent is carried out at a temperature of about 0 to 25° C.

Embodiment P25. The method of embodiment P24, wherein the reaction of the compound of Formula (5) with the deprotecting agent is carried out at a temperature of about 15° C.

Embodiment P26. The method of any one of embodiments P13-P25, wherein the compound of Formula (5) is prepared by reacting a compound of Formula (3):

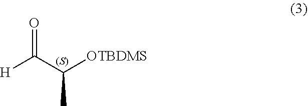

with a compound of Formula (4):

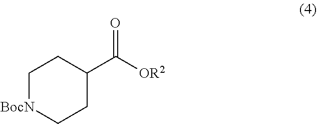

wherein R² is C₁-C₆ alkyl,
to form the compound of Formula (5).

Embodiment P27. The method of embodiment P26, wherein the reaction of the compound of Formula (3) with the compound of Formula (4) further comprises a base.

Embodiment P28. The method of embodiment P27, wherein the base is lithium diisopropylamide (LDA), lithium bis(trimethylsilyl)amide (LiHMDS), lithium tetramethylpiperidide (LiTMP), sodium bis(trimethylsilyl)amide (NaHMDS), or potassium bis(trimethylsilyl)amide (KHMDS).

Embodiment P29. The method of embodiment P28, wherein the base is lithium diisopropylamide (LDA).

Embodiment P30. The method of any one of embodiments P26-P29, wherein the reaction of the compound of Formula (3) with the compound of Formula (4) is carried out using an aprotic solvent.

Embodiment P31. The method of embodiment P30, wherein the aprotic solvent used for the reaction of the compound of Formula (3) with the compound of Formula (4) is tetrahydrofuran, 2-methyltetrahydrofuran, acetonitrile, 1,4-dioxane, toluene, dichloromethane, dichloroethane, chloroform, or a mixture thereof.

Embodiment P32. The method of embodiment P31, wherein the aprotic solvent used for the reaction of the compound of Formula (3) with the compound of Formula (4) is tetrahydrofuran.

Embodiment P33. The method of any one of embodiments P26-P32, wherein the reaction of the compound of Formula (3) with the compound of Formula (4) is carried out at a temperature of about 0 to −40° C.

Embodiment P34. The method of embodiment P33, wherein the reaction of the compound of Formula (3) with the compound of Formula (4) is carried out at a temperature of about −15° C.

Embodiment P35. The method of any one of embodiments P26-P34, wherein the compound of Formula (3) is prepared by reacting a compound of Formula (2):

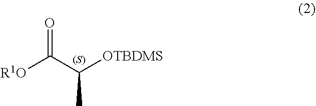

wherein R¹ is C₁-C₆ alkyl,
with a reducing agent to form the compound of Formula (3).

Embodiment P36. The method of embodiment P35, wherein R¹ is methyl or ethyl.

Embodiment P37. The method of embodiment P35 or P36, wherein the reducing agent used for the reaction of the compound of Formula (2) is an organoaluminum hydride, an aluminum hydride, an organoborane hydride, or a borohydride reagent.

Embodiment P38. The method of any one of embodiments P35-P37, wherein the reducing agent used for the reaction of the compound of Formula (2) is diisobutylaluminum hydride (DIBAL-H), sodium bis(2-methoxyethoxy)aluminium hydride (Red-Al), LiAlH₄, LiBHEt₃, L-selectride, N-selectride, K-selectride, sodium borohydride, lithium borohydride, calcium borohydride, or potassium borohydride.

Embodiment P39. The method of embodiment P38, wherein the reducing agent used for the reaction of the compound of Formula (2) is diisobutylaluminum hydride (DIBAL-H).

Embodiment P40. The method of any one of embodiments P35-P39, wherein the reaction of the compound of Formula (2) with the reducing agent is carried out using an aprotic solvent.

Embodiment P41. The method of embodiment P40, wherein the aprotic solvent used for the reaction of the compound of Formula (2) with the reducing agent is toluene, methyl tert-butyl ether (MBTE), cyclopentylmethylether, tetrahydrofuran, 2-methyltetrahydrofuran, acetonitrile, 1,4-dioxane, dichloromethane, dichloroethane, chloroform, n-hexane, cyclohexane, or a mixture thereof.

Embodiment P42. The method of embodiment P41, wherein the aprotic solvent used for the reaction of the compound of Formula (2) with the reducing agent is toluene, methyl tert-butyl ether (MBTE), or a mixture thereof.

Embodiment P43. The method of any one of embodiments P35-P42, wherein the reaction of the compound of Formula (2) with the reducing agent is carried out at a temperature of about −20 to −80° C.

Embodiment P44. The method of embodiment P43, wherein the reaction of the compound of Formula (2) with the reducing agent is carried out at a temperature of about −30 to −60° C.

Embodiment P45. The method of any one of embodiments P35-P44, wherein the compound of Formula (2) is prepared by reacting a compound of Formula (1):

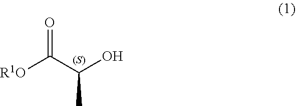

wherein R¹ is C₁-C₆ alkyl,
with TBDMS-X, wherein X is a halide,
to form the compound of Formula (2).

Embodiment P46. The method of embodiment P45, wherein TBDMS-X is TBDMSCl or TBDMSBr.

Embodiment P47. The method of embodiment P45 or P46, wherein TBDMS-X is TBDMSCl.

Embodiment P48. The method of any one of embodiments P45-P47, wherein the reaction of the compound of Formula (1) with TBDMS-X further comprises a base.

Embodiment P49. The method of embodiment P48, wherein the base used for the reaction of Formula (1) with TBDMS-X is NaOH, KOH, LiOH, NaHCO₃, K₂CO₃, or imidazole.

Embodiment P50. The method of embodiment P49, wherein the base used for the reaction of Formula (1) with TBDMS-X is imidazole.

Embodiment P51. The method of any one of embodiments P45-P50, wherein the reaction of the compound of Formula (1) with TBDMS-X is carried out using an aprotic solvent.

Embodiment P52. The method of embodiment P51, wherein the aprotic solvent used for the reaction of the compound of Formula (1) with TBDMS-X is tetrahydrofuran, 2-methyltetrahydrofuran, acetonitrile, 1,4-dioxane, toluene, dichloromethane, dichloroethane, chloroform, or a mixture thereof.

Embodiment P53. The method of embodiment P52, wherein the aprotic solvent used for the reaction of the compound of Formula (1) with TBDMS-X is toluene.

Embodiment P54. The method of any one of embodiments P45-P53, wherein the reaction of the compound of Formula (1) with TBDMS-X is carried out at a temperature of about −10 to 50° C.

Embodiment P55. The method of embodiment P54, wherein the reaction of the compound of Formula (1) with TBDMS-X is carried out at a temperature of about 0 to 25° C. Embodiment P56. A method of preparing a compound of Formula (8):

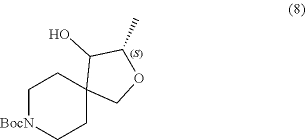

comprising reacting the compound of Formula (7) prepared according to the method of any one of embodiments P1-P55 with a sulfonyl chloride or an acid chloride, and a base to form the compound of Formula (8).

Embodiment P57. The method of embodiment P56, wherein the sulfonyl chloride or the acid chloride is an arylsulfonyl chloride, an alkylsulfonyl chloride, or an aryl acid chloride.

Embodiment P58. The method of embodiment P56 or P57, wherein the sulfonyl chloride or the acid chloride is p-toluenesulfonylchloride, methanesulfonyl chloride, 4-bromo-benzenesulfonyl chloride, 4-nitro-benzenesulfonyl chloride, 1-naphthoyl chloride, or 2-naphthoyl chloride.

Embodiment P59. The method of embodiment P58, wherein the sulfonyl chloride is p-toluenesulfonylchloride.

Embodiment P60. The method of any one of embodiments P56-P59, wherein the base used for the reaction of Formula (7) with the sulfonyl chloride or the acid chloride is NaOH, KOH, LiOH, Ca(OH) 2, CsOH, NaHCO₃, K₂CO₃, or Cs₂CO₃.

Embodiment P61. The method of embodiment P60, wherein the base used for the reaction of Formula (7) with the sulfonyl chloride or the acid chloride is NaOH.

Embodiment P62. The method of any one of embodiments P59-P61, wherein the reaction of Formula (7) with the sulfonyl chloride or the acid chloride and the base is carried out using a mixture of water and an aprotic solvent.

Embodiment P63. The method of embodiment P62, wherein the aprotic solvent used for the reaction of the compound of Formula (7) with the sulfonyl chloride or the acid chloride and the base is methyl tert-butyl ether (MBTE), toluene, cyclohexane, n-heptane, diisopropylether, cyclopentylmethylether, tetrahydrofuran, 2-methyltetrahydrofuran, acetonitrile, 1,4-dioxane, dichloromethane, dichloroethane, chloroform, or a mixture thereof.

Embodiment P64. The method of embodiment P63, wherein the aprotic solvent used for the reaction of the compound of Formula (7) with the sulfonyl chloride or the acid chloride and the base is methyl tert-butyl ether (MBTE), toluene, cyclohexane, n-heptane, diisopropylether, or cyclopentylmethylether.

Embodiment P65. The method of any one of embodiments P56-P64, wherein the reaction of the compound of Formula (7) with the sulfonyl chloride or the acid chloride and the base is carried out at a temperature of about −10 to 30° C.

Embodiment P66. The method of embodiment P65, wherein the reaction of the compound of Formula (7) with the sulfonyl chloride or the acid chloride and the base is carried out at a temperature of about 0° C.

Embodiment P67. A method of preparing a compound of Formula (9):

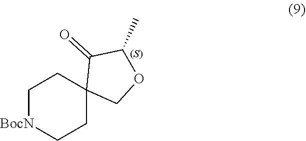

comprising reacting the compound of Formula (8) prepared according to the method of any one of embodiments P56-P66 with an oxidizing agent to form the compound of Formula (9).

Embodiment P68. The method of embodiment P67, wherein the oxidizing agent is Dess-Martin periodinane.

Embodiment P69. The method of embodiment P67, wherein the oxidizing agent is (2,2,6,6-tetramethylpiperidine-1-yl)oxyl (TEMPO).

Embodiment P70. The method of embodiment P67, wherein the oxidizing agent is sulfur trioxide pyridine complex.

Embodiment P71. The method of embodiment P69, wherein the reaction of the compound of Formula (8) with TEMPO further comprises (diacetoxyiodo)benzene or sodium hypochlorite.

Embodiment P72. The method of embodiment P71, wherein the reaction of the compound of Formula (8) with TEMPO further comprises a salt.

Embodiment P73. The method of embodiment P72, wherein the salt is KBr, KCl, or KI.

Embodiment P74. The method of embodiment P73, wherein the salt is KBr.

Embodiment P75. The method of any one of embodiments P67-P74, wherein the reaction of the compound of Formula (8) with the oxidizing agent is carried out using an aprotic solvent or a mixture of an aprotic solvent and water.

Embodiment P76. The method of embodiment P75, wherein the reaction of the compound of Formula (8) with the oxidizing agent is carried out a mixture of the aprotic solvent and water.

Embodiment P77. The method of embodiment P75 or P76, wherein the aprotic solvent used for the reaction of the compound of Formula (8) with the oxidizing agent is tetrahydrofuran, 2-methyltetrahydrofuran, acetonitrile, 1,4-dioxane, dichloromethane, dichloroethane, chloroform, or a mixture thereof.

Embodiment P78. The method of any one of embodiments P75-P77, wherein the aprotic solvent used for the reaction of the compound of Formula (8) with the oxidizing agent is dichloromethane or acetonitrile.

Embodiment P79. The method of any one of embodiments P67-P78, wherein the reaction of the compound of Formula (8) with the oxidizing agent is carried out at a temperature of about −10 to 30° C.

Embodiment P80. The method of embodiment P79, wherein the reaction of the compound of Formula (8) with the oxidizing agent is carried out at a temperature of about 0 to 25° C.

Embodiment P81. The method of any one of embodiments P67-P80, wherein the compound of Formula (9) is prepared without use of column chromatography.

Embodiment P82. A method of preparing a compound of Formula (10) or a salt thereof:

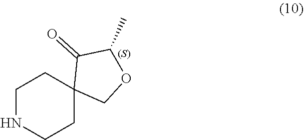

comprising reacting the compound of Formula (9) prepared according to the method of any one of embodiments P67-P81 with an acid to form the compound of Formula (10) or a salt thereof.

Embodiment P83. The method of embodiment P82, wherein the acid is HCl, oxalic acid, phosphoric acid, trifluoroacetic acid, formic acid, HBr, or methanesulfonic acid.

Embodiment P84. The method of embodiment P82 or P83, wherein the acid is HCl.

Embodiment P85. The method of any one of embodiments P82-P84, wherein the salt is an HCl, oxalic acid, phosphoric acid, trifluoroacetic acid, formic acid, HBr, or methanesulfonic acid salt.

Embodiment P86. The method of embodiment P85, wherein the salt is an HCl salt.

Embodiment P87. The method of any one of embodiments P82-P86, wherein the reaction of the compound of Formula (9) with the acid is carried out using an aprotic solvent or a mixture of an aprotic solvent and a protic solvent.

Embodiment P88. The method of embodiment P87, wherein the aprotic solvent used for the reaction of the compound of Formula (9) with the acid is acetone, acetonitrile, tetrahydrofuran, 2-methyltetrahydrofuran, 1,4-dioxane, or a mixture thereof.

Embodiment P89. The method of embodiment P88, wherein the aprotic solvent used for the reaction of the compound of Formula (9) with the acid is acetone.

Embodiment P90. The method of any one of embodiments P87-P89, wherein the protic solvent is an alcohol.

Embodiment P91. The method of embodiment P90, wherein the alcohol is methanol, ethanol, isopropanol, or a mixture thereof.

Embodiment P92. The method of any one of embodiments P82-P91, wherein the reaction of the compound of Formula (9) with the acid is carried out at a temperature of about −10 to 30° C.

Embodiment P93. The method of embodiment P92, wherein the reaction of the compound of Formula (9) with the acid is carried out at a temperature of about 0 to 25° C.

Embodiment P94. A method of preparing a compound of Formula (10)⋅HCl comprising the following steps:

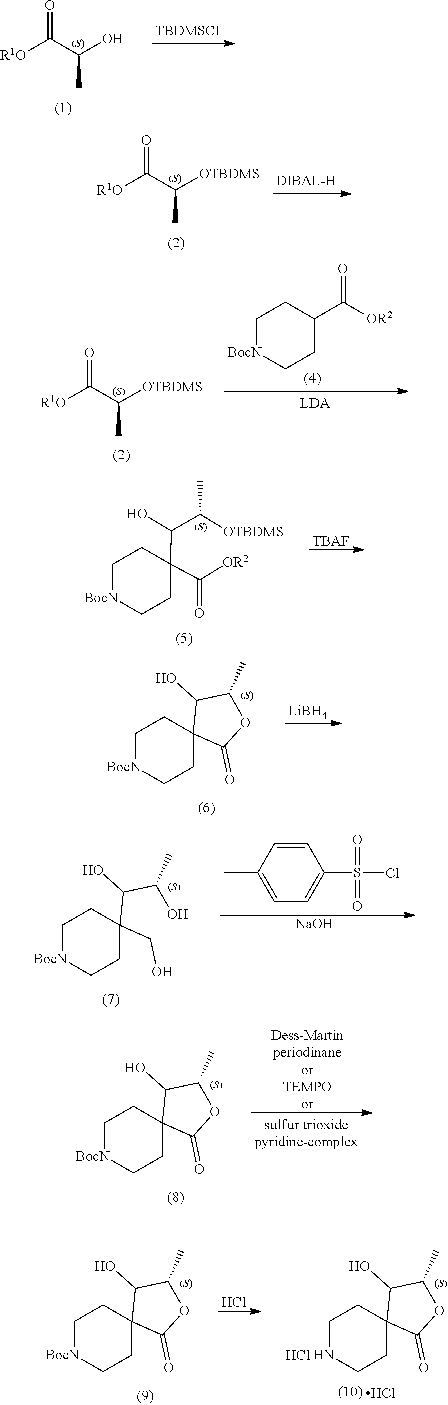

wherein R¹ and R² are each independently methyl or ethyl.

Embodiment P95. The method of any one of embodiments P82-P94, wherein the compound of Formula (10), or a salt thereof, is prepared without use of column chromatography.

Embodiment P96. A method of preparing a compound of Formula (11):

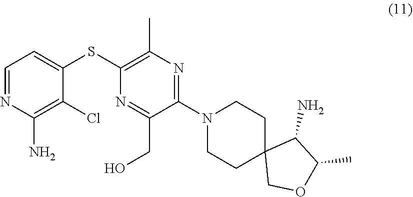 [SMILES: Cc1nc(N2CCC3(CC2)CO[C@@H](C)[C@H]3N)c(CO)nc1Sc1ccnc(N)c1Cl]

or a salt thereof, comprising converting the compound of Formula (10), or a salt thereof, prepared according to any one of embodiments P82-P95 to the compound of Formula (11) or a salt thereof.

Embodiment P97. A compound of Formula (6):

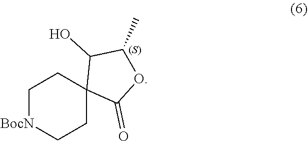

Embodiment P98. The compound of embodiment P97, which is of Formula (6a):

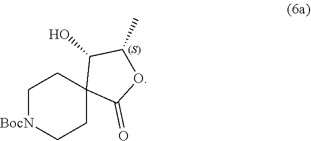

Embodiment P99. The compound of embodiment P97, which is of Formula (6b):

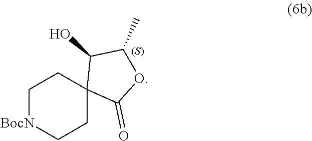

### EXAMPLES

The examples and preparations provided below further illustrate and exemplify the compounds and synthetic methods of the present disclosure. It is to be understood that the scope of the present disclosure is not limited in any way by the scope of the following examples.

The following abbreviations may be relevant for the application.

### Abbreviations

ACN: acetonitrile
aq.: aqueous
Boc: tert-butyloxycarbonyl
Bu: butyl
DCM: dichloromethane
DIBAL-H: diisobutylaluminium hydride
DIPE: diisopropylether
DIPEA: diisopropylethylamine
DMF: N,N-dimethylformamide
DMAP: 4-dimethylaminopyridine
DMSO: dimethylsulfoxide
EA or EtOAc: ethyl acetate
EP: expected product
eq. or equiv.: equivalents
Et: ethyl
EtOH: ethanol
FA: formic acid
FCC: flash column chromatography
GC: gas chromatography
h or hr: hour(s)
HPLC: high-performance liquid chromatography
LCMS: liquid chromatography mass spectrometry
LDA: lithium diisopropylamide
Me: methyl
MeOH: methanol
MeTHF or 2-MeTHF: 2-methyltetrahydrofuran
MTBE: methyl tert-butyl ether
NBS: N-bromosuccinimide
OAc: acetate
PE: petroleum ether
rt: retention time
RT: room temperature
sat.: saturated
TBAF: tetra-n-butylammonium fluoride
TBDMSCl: tert-butyldimethylsilyl chloride
TEMPO: (2,2,6,6-Tetramethylpiperidin-1-yl)oxyl
THF: tetrahydrofuran
TLC: thin layer chromatography
vol: volume(s)
Xantphos: 4,5-bis(diphenylphosphino)-9,9-dimethylxanthene

Examples 1-8 provide synthetic details for preparing Compound (10), or a salt thereof, according to the general reaction scheme shown below, wherein R¹ and R² are each independently C₁-C₆ alkyl.

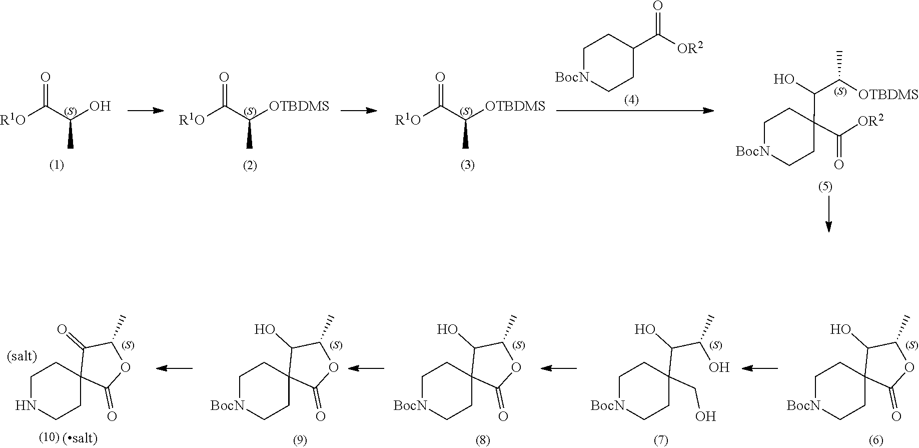

### Example 1a. Synthesis of Ethyl(S)-2-((tert-butyldimethylsilyl)oxy) propanoate (2a)

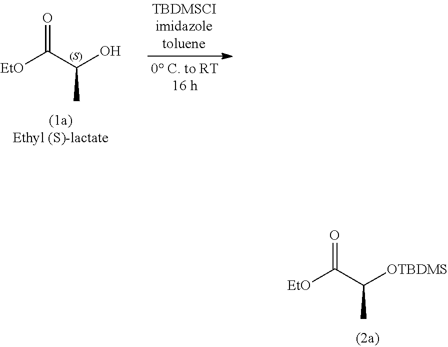

Condition 1. To a solution of ethyl(S)-lactate (10.0 g, 84.7 mmol) in toluene (70 mL) was added imidazole (6.3 g, 93.1 mmol) and the suspension was stirred until a clear solution was obtained. Then the mixture was cooled to 0° C. and solid TBDMSCl (14.0 g, 93.1 mmol) was added portionwise maintaining the temperature below 25° C. Once the addition was complete, the suspension was stirred at 25° C. for 16 h before being cooled down to 10° C. and quenched with water (30 mL). After phase separation, the organic layer was further washed with water (2×30 mL) before being concentrated to give 18.9 g of ethyl(S)-2-((tert-butyldimethylsilyl)oxy) propanoate (2a) as a colorless oil (96% yield).

Condition 2. To a solution of ethyl(S)-lactate (12.0 g, 101.6 mmol) in toluene (50 mL), imidazole (7.6 g, 111.7 mmol) was added and the suspension was stirred until a clear solution was obtained. Then the mixture was cooled to 0° C. and 50% w/w TBDMSCl in toluene (38.8 mL, 111.7 mmol) was added dropwise, maintaining the temperature below 25° C. Once the addition was complete, the suspension was stirred at 25° C. for 16 h before being cooled down to 10° C. and quenched with water (30 mL). After phase separation, the organic layer was further washed with water (2×30 mL) before being concentrated to give 22.4 g of ethyl(S)-2-((tert-butyldimethylsilyl)oxy) propanoate (2a) as a colorless oil (95% yield). ¹H NMR (400 MHZ, DMSO-d6) δ ppm 4.33 (q, 1H), 4.13 (q, 1H), 1.27 (d, 3H), 1.19 (t, 3H), 0.88 (s, 9H), 0.10 (s, 6H).

### Example 1b. Synthesis of Methyl(S)-2-((tert-butyldimethylsilyl)oxy) propanoate (2b)

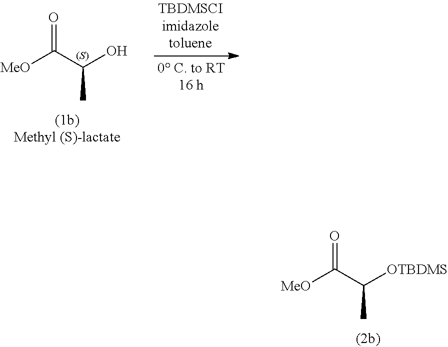

To a solution of methyl(S)-lactate (10.0 g, 96.1 mmol) in toluene (70 mL) was added imidazole (7.2 g, 105.7 mmol) and the suspension was stirred until a clear solution was obtained. Then the mixture was cooled to 0° C. and 50% w/w TBDMSCl in toluene (36.8 mL, 105.7 mmol) was added dropwise, maintaining the temperature below 25° C. Once the addition was complete, the suspension was stirred at 25° C. for 16 h before being cooled down to 10° C. and quenched with water (30 mL). After phase separation, the organic layer was further washed with water (2×30 mL) before being concentrated to give 19.9 g of methyl(S)-2-((tert-butyldimethylsilyl)oxy) propanoate (2b) as a colorless oil (95% yield).

### Example 2. Synthesis of(S)-2-((tert-butyldimethylsilyl)oxy) propanal (3)

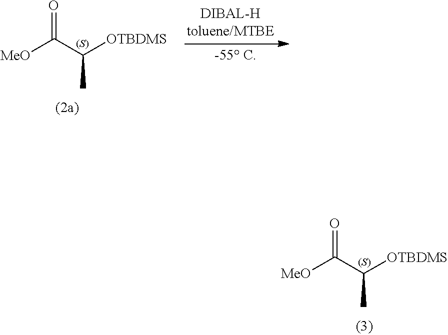

Condition 1. A solution of ethyl(S)-2-((tert-butyldimethylsilyl)oxy) propanoate (232.4 g/L in MTBE) and 20% w/w DIBAL-H in toluene solution was pumped into a Continous Stirred Tank Reactor (CSTR). The reaction temperature was set between −56° C. and −40° C. and the residence time was set between 2 and 9 minutes. The reaction was performed with a slight excess of DIBAL-H, from 1.05 to 1.3 equivalents versus ethyl(S)-2-((tert-butyldimethylsilyl)oxy) propanoate. The CSTR output was quenched with a 20% w/w aqueous solution of Rochelle salt. After phase separation, the aqueous phase was counter-extracted with MTBE and the combined organic layers were concentrated to dryness to give(S)-2-((tert-butyldimethylsilyl)oxy) propanal (3) as a colorless oil (80% yield).

Condition 2. A solution of ethyl(S)-2-((tert-butyldimethylsilyl)oxy) propanoate (232.4 g/L in toluene) and 20% w/w DIBAL-H in toluene solution were pumped into a Continous Stirred Tank Reactor (CSTR). The reaction temperature was set between −56° C. and −40° C. and the residence time was set between 2 and 9 minutes. The reaction was performed with a slight excess of DIBAL-H, from 1.05 to 1.3 equivalents versus ethyl(S)-2-((tert-butyldimethylsilyl)oxy) propanoate. The CSTR output was quenched with a 20% w/w aqueous solution of Rochelle salt. After phase separation, the aqueous phase was counter-extracted with toluene and the combined organic layers were concentrated to dryness to give(S)-2-((tert-butyldimethylsilyl)oxy) propanal (3) as a colorless oil (82% yield).

Condition 3. Three series of Continous Stirred Tank Reactors (CSTRs) were used to perform the reduction of ethyl(S)-2-((tert-butyldimethylsilyl)oxy) propanoate. A solution of ethyl(S)-2-((tert-butyldimethylsilyl)oxy) propanoate (46.5 g/L in MTBE) was continuously pumped into the first CSTR, which was cooled down to −40° C. The cold reagent was reacted with 20% w/w DIBAL-H in toluene solution in the second CSTR (reaction temperature: from −56° C. to −40° C., residence time: from 2 to 9 minutes, DIBAL-H equivalents from 1.05 to 1.3). The reaction mixture was continuously quenched with a 20% w/w aqueous solution of Rochelle salt in the third CSTR (quenching temperature: 0-5° C.). After phase separation, the aqueous phase was counter-extracted with MTBE and the combined organic layers were concentrated to dryness to give(S)-2-((tert-butyldimethylsilyl)oxy) propanal (3) as a colorless oil (83% yield).

Condition 4. A solution of ethyl(S)-2-((tert-butyldimethylsilyl)oxy) propanoate (20.0 g, 86.1 mmol) in MTBE (200 mL) was cooled down to −55° C. Once this temperature was reached, 25% w/w DIBAL-H in toluene solution (69.5 mL, 103.3 mmol) was carefully added not to exceed −50° C., and the mixture was stirred for 15 min before being quenched with a 20% w/w aqueous solution of Rochelle salt (100 mL). After phase separation, the aqueous phase was counter-extracted with MTBE (2×100 mL) and the combined organic layers were concentrated to dryness to give 13.8 g of(S)-2-((tert-butyldimethylsilyl)oxy) propanal (3) as a colorless oil (85% yield).

Condition 5. A solution of ethyl(S)-2-((tert-butyldimethylsilyl)oxy) propanoate (10 g, 43.1 mmol) in toluene (100 mL) was cooled down to −55° C. Once this temperature was reached, 25% w/w DIBAL-H in toluene solution (34.8 mL, 51.7 mmol) was carefully added not to exceed −50° C., and the mixture was stirred for 15 min before being quenched with a 20% w/w aqueous solution of Rochelle salt (50 mL). After phase separation, the aqueous phase was counter-extracted with MTBE (2×50 mL) and the combined organic layers were concentrated to dryness to give 6.7 g of(S)-2-((tert-butyldimethylsilyl)oxy) propanal (3) as a colorless oil (82% yield). ¹H NMR (400 MHZ, CDCl₃) δ ppm 9.61 (s, 1H), 4.09 (q, 1H), 1.28 (d, 3H), 0.92 (s, 9H), 0.10 (s, 6H).

### Example 3. Synthesis of 1-(tert-butyl) 4-ethyl 4-((2S)-2-((tert-butyldimethylsilyl)oxy)-1-hydroxypropyl) piperidine-1,4-dicarboxylate (5)

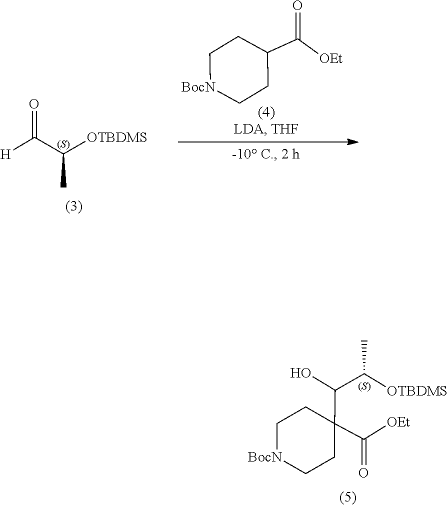

A solution of diisopropylamine (5.0 g, 49.4 mmol) in THF (50 mL) was cooled to −30° C. and 1.6 M buthyllithium in hexanes (29.5 mL, 46.9 mmol) was added dropwise. After the addition, the solution was warmed up to 0° C. and stirred further for 30 min. After this time, the mixture was cooled down to −10° C. and 1-tert-butyl 4-ethyl piperidine-1,4-dicarboxylate (11.6 g, 45.0 mmol) was added dropwise over 2 hours, without exceeding −5° C. Once the addition was complete, the mixture was stirred at −5° C. for 1 additional hour at −5° C. before(S)-2-((tert-butyldimethylsilyl)oxy) propanal (7.7 g, 41.0 mmol) was added. The resulting mixture was then warmed up to 20° C. and stirred for 1 hour before being quenched with aq. sat. ammonium chloride (30 mL). After phase separation, the aqueous layer was counter-extracted with MTBE (1×30 mL) and the combined organic layers were concentrated to remove the THF, before being washed with 5% w/w aq. citric acid (30 mL), 8% w/w aq. sodium bicarbonate (30 mL), and saturated brine (30 mL). The resulting MTBE phase was then concentrated to give 12.8 g of 1-(tert-butyl) 4-ethyl 4-((2S)-2-((tert-butyldimethylsilyl)oxy)-1-hydroxypropyl) piperidine-1,4-dicarboxylate (5) as a yellowish oil (70% yield).

### Example 4. Tert-butyl (3S)-4-hydroxy-3-methyl-1-oxo-2-oxa-8-azaspiro[4.5]decane-8-carboxylate (6)

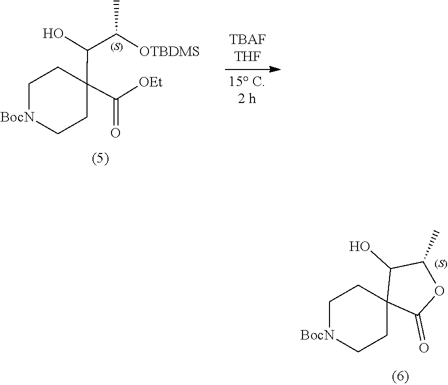

Condition 1. A solution of 1-(tert-butyl) 4-ethyl 4-((2S)-2-((tert-butyldimethylsilyl)oxy)-1-hydroxypropyl) piperidine-1,4-dicarboxylate (15.0 g, 33.7 mmol) in THF (70 mL) was cooled down to 15° C. and 1 M tetrabutylammonium fluoride in THF (37.1 mL, 37.1 mmol) was added over 1 hour. The mixture was stirred for 1 additional hour at 15° C. and quenched with 8% w/w aq. sodium bicarbonate (30 mL). After phase separation, the aqueous phase was counter-extracted with isopropylacetate (3×30 mL) and the combined organic phase was concentrated to remove THF. The resulting isopropylacetate phase was washed with water (2×30 mL) to remove excess TBAF and concentrated until the precipitation of the solid tert-butyl (3S)-4-hydroxy-3-methyl-1-oxo-2-oxa-8-azaspiro[4.5]decane-8-carboxylate (6). The suspension was further concentrated to 30 mL, combined with n-heptane (30 mL), cooled down to −20° C., and filtered to afford 8.2 g of (6) as a white crystalline solid (85% yield as 98:2 diastereoisomeric mixture).

Condition 2. A solution of 1-(tert-butyl) 4-ethyl 4-((2S)-2-((tert-butyldimethylsilyl)oxy)-1-hydroxypropyl) piperidine-1,4-dicarboxylate (10.0 g, 22.5 mmol) in THF (50 mL) was cooled down to 15° C. and 1 M tetrabutylammonium fluoride in THF (24.7 mL, 24.7 mmol) was added over 1 hour. The mixture was stirred for 1 additional hour at 15° C. and quenched with 8% w/w aq. sodium bicarbonate (20 mL). After phase separation, the aqueous phase was counter-extracted with 2-MeTHF (3×20 mL) and the combined organic phase was concentrated to remove THF. The resulting 2-MeTHF phase was washed with water (2×20 mL) to remove excess TBAF and concentrated until the precipitation of the solid tert-butyl (3S)-4-hydroxy-3-methyl-1-oxo-2-oxa-8-azaspiro[4.5]decane-8-carboxylate (6). The suspension was further concentrated to 20 mL, cooled down to −20° C., and filtered to afford 5.3 g of (6) as a white crystalline solid (83% yield as 98:2 diastereoisomeric mixture).

Condition 3. A solution of 1-(tert-butyl) 4-ethyl 4-((2S)-2-((tert-butyldimethylsilyl)oxy)-1-hydroxypropyl) piperidine-1,4-dicarboxylate (10.0 g, 22.5 mmol) in THF (50 mL) was cooled down to 15° C. and 1 M tetrabutylammonium fluoride in THF (24.7 mL, 24.7 mmol) was added over 1 hour. The mixture was stirred for 1 additional hour at 15° C. and quenched with 8% w/w aq. sodium bicarbonate (20 mL). After phase separation, the aqueous phase was counter-extracted with toluene (3×20 mL) and the combined organic phase was concentrated to remove THF. The resulting toluene phase was washed with water (2×20 mL) to remove excess TBAF and concentrated until the precipitation of the solid tert-butyl (3S)-4-hydroxy-3-methyl-1-oxo-2-oxa-8-azaspiro[4.5]decane-8-carboxylate (6). The suspension was further concentrated to 40 mL, cooled down to −20° C., and filtered to afford 5.3 g of (6) as a white crystalline solid (82% yield as 96:4 diastereoisomeric mixture).

Condition 4. A solution of 1-(tert-butyl) 4-ethyl 4-((2S)-2-((tert-butyldimethylsilyl)oxy)-1-hydroxypropyl) piperidine-1,4-dicarboxylate (12.0 g, 27.0 mmol) in THF (50 mL) was cooled down to 15° C. and 1 M tetrabutylammonium fluoride in THF (29.7 mL, 29.7 mmol) was added over 1 hour. The mixture was stirred for 1 additional hour at 15° C. and quenched with 8% w/w aq. sodium bicarbonate (20 mL). After phase separation, the aqueous phase was counter-extracted with dichloromethane (3×20 mL) and the combined organic phase was concentrated to remove THF. The resulting dichloromethane phase was washed with water (2×20 mL) to remove excess TBAF and added with MTBE (40 mL) until the precipitation of the solid tert-butyl (3S)-4-hydroxy-3-methyl-1-oxo-2-oxa-8-azaspiro[4.5]decane-8-carboxylate (6). The suspension was further concentrated to 20 mL, cooled down to −20° C., and filtered to afford 6.5 g of (6) as a white crystalline solid (84% yield as 97:3 diastereoisomeric mixture).

Condition 5. A solution of 1-(tert-butyl) 4-ethyl 4-((2S)-2-((tert-butyldimethylsilyl)oxy)-1-hydroxypropyl) piperidine-1,4-dicarboxylate (10.0 g, 22.5 mmol) in THF (50 mL) was cooled down to 15° C. and 1 M tetrabutylammonium fluoride in THF (24.7 mL, 24.7 mmol) was added over 1 hour. The mixture was stirred for 1 additional hour at 15° C. and quenched with 8% w/w aq. sodium bicarbonate (20 mL). After phase separation, the aqueous phase was counter-extracted with ethyl acetate (3×20 mL) and the combined organic phase was concentrated to remove THF. The resulting ethyl acetate phase was washed with water (2×20 mL) to remove excess TBAF and concentrated until the precipitation of the solid tert-butyl (3S)-4-hydroxy-3-methyl-1-oxo-2-oxa-8-azaspiro[4.5]decane-8-carboxylate (6). The suspension was further concentrated to 30 mL, cooled down to −20° C., and filtered to afford 5.6 g of (6) as a white crystalline solid (87% yield as 95:5 diastereoisomeric mixture).

Characterization data of Tert-butyl (3S)-4-hydroxy-3-methyl-1-oxo-2-oxa-8-azaspiro[4.5]decane-8-carboxylate (6). Reversed-phase HPLC (column: Waters Xbridge C18, 150*4.6 mm, 3.5 μm, UV 200 nm, flow rate: 1 mL/min, mobile phase (A: water (0.1% FA), B: acetonitrile (0.1% FA)), gradient: 4 min 5% B, 20 min 80% B) of a 96.2:3.8 diastereomer mixture gave two peaks with rt 13.588 min (3.83%) and 14.152 min (96.17%). Reversed-phase chiral HPLC (column: AY-H Chiralpak, 150*4.6 mm, 5 μm, UV 210 nm, flow rate: 2 mL/min, mobile phase (A: n-hexane, B: isopropyl alcohol, 10% B) of a 98.7:1.3 diastereomer mixture gave two peaks with rt 3.307 min (98.53%) and 4.511 min (1.47%). ¹H NMR (400 MHZ, DMSO-d6) δ ppm 5.78 (d, 1H), 4.22 (m, 1H), 3.68 (m, 2H), 3.55 (m, 2H), 3.15 (m, 1H), 1.75 (m, 1H), 1.65 (m, 1H), 1.55 (m, 2H), 1.4 (s, 9H), 1.35 (d, 3H).

### Example 5. Tert-butyl 4-((2S)-1,2-dihydroxypropyl)-4-(hydroxymethyl) piperidine-1-carboxylate (7)

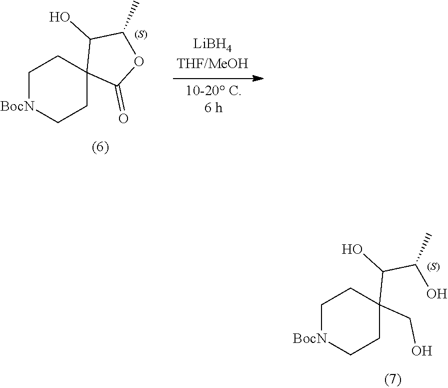

Condition 1. A solution of tert-butyl (3S)-4-hydroxy-3-methyl-1-oxo-2-oxa-8-azaspiro[4.5]decane-8-carboxylate (20.0 g, 70.1 mmol) in THF (120 mL) was cooled down to 10° C. and 4 M LiBH₄ in THF (35.1 mL, 140.2 mmol) was added dropwise over 3 hours. Once the addition was complete, the mixture was added with MeOH (20 mL) and stirred for additional 3 h at 20° C. After this time, the mixture was gently poured on an aqueous citrate buffer pH 3.5 (60 mL) at 5° C. and stirred for 1 additional hour. Then THF was removed by distillation and the aqueous phase was extracted with 2-MeTHF (3×60 mL). The combined organic layers were washed with 8% w/w aq. sodium bicarbonate (30 mL) and saturated brine (30 mL), and concentrated to afford 19.3 g of tert-butyl 4-((2S)-1,2-dihydroxypropyl)-4-(hydroxymethyl) piperidine-1-carboxylate (7) as a colorless viscous oil (95% yield).

Condition 2. A solution of tert-butyl (3S)-4-hydroxy-3-methyl-1-oxo-2-oxa-8-azaspiro[4.5]decane-8-carboxylate (10.0 g, 35.0 mmol) in THF (40 mL)/MeOH (20 mL) was cooled down to 10° C. and 4 M LiBH₄ in THF (17.5 mL, 70.0 mmol) was added dropwise over 3 hours. After stirring at 20° C. for 3 additional hours, the mixture was gently poured on a 20% w/w aqueous acetic acid solution (20 mL) at 5° C. and stirred for 1 additional hour. Then THF was removed by distillation and the aqueous phase was extracted with 2-MeTHF (3×30 mL). The combined organic layers were washed with 8% w/w aq. sodium bicarbonate (20 mL) and saturated brine (20 mL), and concentrated to afford 9.4 g of tert-butyl 4-((2S)-1,2-dihydroxypropyl)-4-(hydroxymethyl) piperidine-1-carboxylate (7) as colorless viscous oil (93% yield). ¹H NMR (400 MHZ, DMSO-d6) δ ppm 4.77 (q, 1H), 4.62 (d, 1H), 4.50 (d, 1H), 3.77 (q, 1H), 3.50 (m, 4H), 3.18 (t, 1H), 3.12 (m, 2H), 1.60 (m, 2H), 1.39 (m, 11H), 1.12 (d, 3H).

### Condition 3.

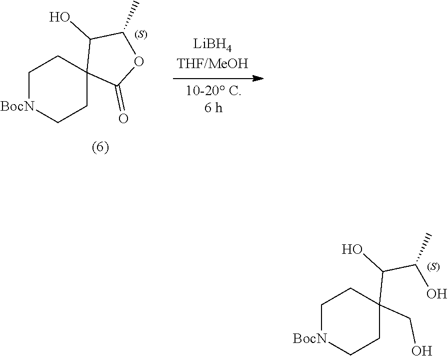

A solution of tert-butyl (3S)-4-hydroxy-3-methyl-1-oxo-2-oxa-8-azaspiro[4.5]decane-8-carboxylate (10.0 g, 35.0 mmol) in THF (40 mL)/EtOH (20 mL) was cooled down to 10° C. and LiBH₄ 4 M in THF (17.5 mL, 70.0 mmol) was added dropwise over 3 hours. After stirring at 20° C. for 3 additional hours, the mixture was gently poured on a aqueous citrate buffer pH 3.5 (20 mL) at 5° C. and stirred for 1 additional hour. Then THF was removed by distillation and the aqueous phase was extracted with 2-MeTHF (3×30 mL). The combined organic layers were washed with 8% w/w aq. sodium bicarbonate (20 mL) and saturated brine (20 mL) and concentrated to afford 9.1 g of tert-butyl 4-((2S)-1,2-dihydroxypropyl)-4-(hydroxymethyl) piperidine-1-carboxylate (7) as a colorless viscous oil (90% yield).

### Example 6. Tert-butyl (3S)-4-hydroxy-3-methyl-2-oxa-8-azaspiro[4.5]decane-8-carboxylate (8)

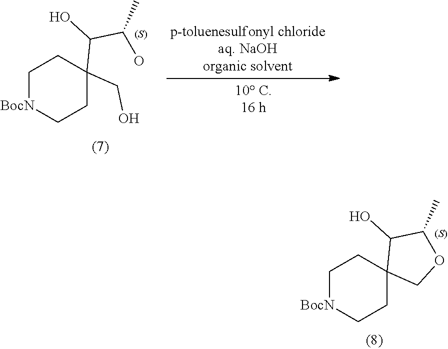

Condition 1. To a solution of tert-butyl 4-((2S)-1,2-dihydroxypropyl)-4-(hydroxymethyl) piperidine-1-carboxylate (15.0 g, 51.8 mmol) in water (450 mL) at 5° C. was added NaOH (8.3 g, 207.2 mmol) followed by a solution of p-toluenesulfonyl chloride (24.7 g, 129.5 mmol) in MTBE (150 mL), and the resulting mixture was stirred for 16 h maintaining the pH above 12 by rinsing with additional NaOH. Following the removal of MTBE by distillation, n-heptane (50 mL) was added and the mixture was stirred for 1 additional hour. After phase separation (organic phase discarded), the aqueous phase was extracted with toluene (3×200 mL) and the combined organic layers were concentrated to give 9.8 g of tert-butyl (3S)-4-hydroxy-3-methyl-2-oxa-8-azaspiro[4.5]decane-8-carboxylate (8) as a colorless viscous oil (70% yield). 1H NMR (400 MHZ, DMSO-d6) δ ppm 3.61 (m, 5H), 3.25 (d, 1H), 2.98 (bs, 2H), 1.61 (m, 1H), 1.35 (m, 12H), 1.18 (d, 3H).

Condition 2. To a solution of tert-butyl 4-((2S)-1,2-dihydroxypropyl)-4-(hydroxymethyl) piperidine-1 carboxylate (12.0 g, 41.4 mmol) in water (350 mL) at 10° C. was added NaOH (6.6 g, 165.8 mmol) followed by a solution of p-toluenesulfonyl chloride (19.8 g, 103.6d mmol) in toluene (120 mL), and the resulting mixture was stirred for 16 h maintaining the pH above 12 by rinsing with additional NaOH. After phase separation, the aqueous phase was counter-extracted with additional toluene (2×120 mL) and the combined toluene layers were concentrated to give 8.2 g of tert-butyl (3S)-4-hydroxy-3-methyl-2-oxa-8-azaspiro[4.5]decane-8-carboxylate (8) as colorless viscous oil (73% yield).

Condition 3. To a solution of tert-butyl 4-((2S)-1,2-dihydroxypropyl)-4-(hydroxymethyl) piperidine-1-carboxylate (10.0 g, 34.5 mmol) in water (300 mL) at 10° C. was added NaOH (5.5 g, 138.2 mmol) followed by a solution of p-toluenesulfonyl chloride (16.5 g, 86.3 mmol) in cyclohexane (300 mL), and the resulting mixture was stirred for 16 h maintaining the pH above 12 by rinsing with additional NaOH. After phase separation (organic phase discarded), the aqueous phase was extracted with toluene (3×150 mL) and the combined organic layers were concentrated to give 5.6 g of tert-butyl (3S)-4-hydroxy-3-methyl-2-oxa-8-azaspiro[4.5]decane-8-carboxylate (8) as colorless viscous oil (60% yield).

Condition 4. To a solution of tert-butyl 4-((2S)-1,2-dihydroxypropyl)-4-(hydroxymethyl) piperidine-1-carboxylate (12.0 g, 41.4 mmol) in water (200 mL) at 10° C. was added NaOH (6.6 g, 165.8 mmol) followed by a solution of p-toluenesulfonyl chloride (19.8 g, 103.6 mmol) in n-heptane (200 mL), and the resulting mixture was stirred for 16 h maintaining the pH above 12 by rinsing with additional NaOH. After phase separation (organic phase discarded), the aqueous phase was extracted with toluene (3×150 mL) and the combined organic layers were concentrated to give 7.6 g of tert-butyl (3S)-4-hydroxy-3-methyl-2-oxa-8-azaspiro[4.5]decane-8-carboxylate (8) as colorless viscous oil (68% yield).

Condition 5. To a solution of tert-butyl 4-((2S)-1,2-dihydroxypropyl)-4-(hydroxymethyl) piperidine-1-carboxylate (8.0 g, 27.6 mmol) in water (200 mL) at 10° C. was added NaOH (4.4 g, 110.5 mmol) followed by a solution of p-toluenesulfonyl chloride (13.2 g, 69.1 mmol) in diisopropylether (200 mL), and the resulting mixture was stirred for 16 h maintaining the pH above 12 by rinsing with additional NaOH. Diisopropylether was removed by distillation and n-heptane (50 mL) was added, and the mixture was stirred for 1 additional hour. After phase separation (organic phase discarded), the aqueous phase was extracted with toluene (3×100 mL) and the combined organic layers were concentrated to give 5.4 g of tert-butyl (3S)-4-hydroxy-3-methyl-2-oxa-8-azaspiro[4.5]decane-8-carboxylate (8) as colorless viscous oil (72% yield).

Condition 6. To a solution of tert-butyl 4-((2S)-1,2-dihydroxypropyl)-4-(hydroxymethyl) piperidine-1-carboxylate (12.0 g, 41.4 mmol) in water (300 mL) at 10° C. was added NaOH (6.6 g, 165.8 mmol) followed by a solution of p-toluenesulfonyl chloride (19.8 g, 103.6 mmol) in cyclopentylmethylether (300 mL), and the resulting mixture was stirred for 16 h maintaining the pH above 12 by rinsing with additional NaOH. Cyclopentylmethylether was removed by distillation and n-heptane (100 mL) was added, and the mixture was stirred for 1 additional hour. After phase separation (organic phase discarded), the aqueous phase was extracted with toluene (3×200 mL) and the combined organic layers were concentrated to give 8.0 g of tert-butyl (3S)-4-hydroxy-3-methyl-2-oxa-8-azaspiro[4.5]decane-8-carboxylate (8) as a colorless viscous oil (71% yield).

Condition 7. To a solution of tert-butyl 4-((2S)-1,2-dihydroxypropyl)-4-(hydroxymethyl) piperidine-1-carboxylate (8.0 g, 27.6 mmol) in water (250 mL) at 10° C. was added NaOH (4.4 g, 110.5 mmol) followed by solid p-toluenesulfonyl chloride (13.2 g, 69.1 mmol) in 5 portions over 6 hours, and the resulting mixture was stirred for 16 h maintaining the pH above 12 by rinsing with additional NaOH. Then n-heptane (50 mL) was added, and the mixture was stirred for 1 additional hour. After phase separation (organic phase discarded), the aqueous phase was extracted with toluene (3×150 mL) and the combined organic layers were concentrated to give 5.3 g of tert-butyl (3S)-4-hydroxy-3-methyl-2-oxa-8-azaspiro[4.5]decane-8-carboxylate (8) as a colorless viscous oil (71% yield).

### Example 7. Tert-butyl(S)-3-methyl-4-oxo-2-oxa-8-azaspiro[4.5]decane-8-carboxylate (9)

### Condition 1.

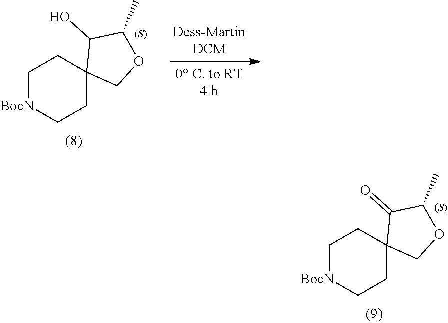

To a solution of tert-butyl (3S)-4-hydroxy-3-methyl-2-oxa-8-azaspiro[4.5]decane-8-carboxylate (5.0 g, 18.4 mmol) in dichloromethane (50 mL) at 0° C., Dess-Martin periodinane (8.2 g, 19.3 mmol) was added portionwise, and the resulting mixture was left to reach RT and stirred further for 4 hours before being quenched with 8% w/w aq. sodium bicarbonate (50 mL) and sodium metabisulfite (4.0 g, 21.0 mmol). After phase separation, the aqueous layer was counter-extracted with dichloromethane (2×20 mL) and the combined organic layers were concentrated to give 4.5 g of tert-butyl(S)-3-methyl-4-oxo-2-oxa-8-azaspiro[4.5]decane-8-carboxylate (9) as a pale yellow viscous oil (90% yield). ¹H NMR (400 MHZ, DMSO-d6) δ ppm 4.23 (d, 1H), 3.925 (d, 1H), 3.77 (d, 1H), 3.70 (m, 2H), 3.12 (bs, 1H), 2.85 (bs, 1H), 1.35 (m, 13H), 1.18 (d, 3H).

### Condition 2.

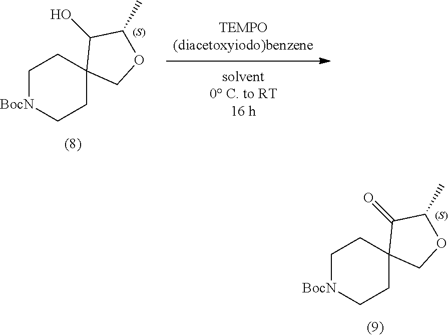

To a solution of tert-butyl (3S)-4-hydroxy-3-methyl-2-oxa-8-azaspiro[4.5]decane-8-carboxylate (10.0 g, 36.8 mmol) in dichloromethane (80 mL) at 0° C., TEMPO (0.6 g, 3.7 mmol) was added followed by (diacetoxyiodo)benzene (12.4 g, 38.6 mmol). The resulting mixture was left to reach RT and stirred further for 16 hours before being quenched with 8% w/w aq. sodium bicarbonate (30 mL) and sodium metabisulfite (8.0 g, 42.0 mmol). After phase separation, the aqueous layer was counter-extracted with dichloromethane (2×30 mL) and the combined organic layers were concentrated to give 8.3 g of tert-butyl(S)-3-methyl-4-oxo-2-oxa-8-azaspiro[4.5]decane-8-carboxylate (9) as a pale yellow viscous oil (84% yield).

Condition 3. To a solution of tert-butyl (3S)-4-hydroxy-3-methyl-2-oxa-8-azaspiro[4.5]decane-8-carboxylate (10.0 g, 36.8 mmol) in acetonitrile (40 mL)/water (40 mL) at 0° C., TEMPO (0.6 g, 3.7 mmol) was added followed by (diacetoxyiodo)benzene (12.4 g, 36.8 mmol). The resulting mixture was left to reach RT and stirred further for 16 hours before being quenched with 8% w/w aq. sodium bicarbonate (30 mL) and sodium metabisulfite (8.0 g, 42.0 mmol). After phase separation, the aqueous layer was counter-extracted with MTBE (2×40 mL) and the combined organic layers were concentrated to give 8.5 g of tert-butyl(S)-3-methyl-4-oxo-2-oxa-8-azaspiro[4.5]decane-8-carboxylate (9) as a pale yellow viscous oil (86% yield).

### Condition 4.

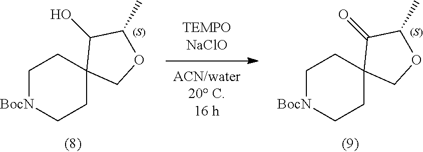

To a solution of tert-butyl (3S)-4-hydroxy-3-methyl-2-oxa-8-azaspiro[4.5]decane-8-carboxylate (15.0 g, 55.2 mmol) in acetonitrile (70 mL)/water (70 mL) at 0° C., TEMPO (0.9 g, 5.6 mmol) was added followed by 10% w/w aq. sodium hypochlorite (45.0 g, 60.7 mmol). The resulting mixture was left to reach RT and stirred further for 16 hours before being quenched with sodium metabisulfite (12.4 g, 65.0 mmol). After phase separation, the aqueous layer was counter-extracted with MTBE (2×70 mL) and the combined organic layers was concentrated to give 12.3 g of tert-butyl(S)-3-methyl-4-oxo-2-oxa-8-azaspiro[4.5]decane-8-carboxylate (9) as a pale yellow viscous oil (83% yield).

### Condition 5.

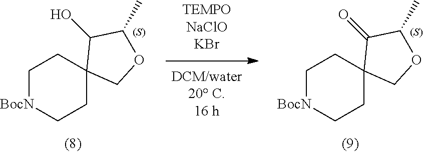

To a solution of tert-butyl (3S)-4-hydroxy-3-methyl-2-oxa-8-azaspiro[4.5]decane-8-carboxylate (6.0 g, 22.1 mmol) in dichloromethane (30 mL)/water (30 mL) at 0° C., TEMPO (0.34 g, 2.2 mmol) was added followed by potassium bromide (2.9 g, 24.3 mmol) and aq. sodium hypochlorite (1.8 g, 24.3 mmol). The resulting mixture was left to reach RT and stirred further for 16 hours before being quenched with sodium metabisulfite (5.1 g, 27.0 mmol). After phase separation, the aqueous layer was counter-extracted with dichloromethane (2×30 mL) and the combined organic layers were concentrated to give 4.8 g of tert-butyl(S)-3-methyl-4-oxo-2-oxa-8-azaspiro[4.5]decane-8-carboxylate (9) as a pale yellow viscous oil (81% yield).

### Condition 6.

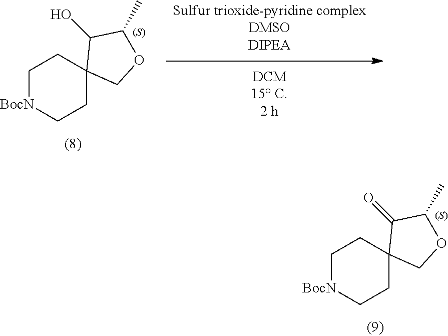

Tert-butyl (3S)-4-hydroxy-3-methyl-2-oxa-8-azaspiro[4.5]decane-8-carboxylate (32.0 g, 118.0 mmol) was dissolved in DCM (640 mL). Diisopropylethylamine (60.9 g, 471.22 mmol) was added and the resulting pale yellow solution was cooled down to 15° C. In another flask, pyridine sulfur trioxide complex (37.6 g, 236.24 mmol) was added portionwise to DMSO (111 g, 1.42 mol) at room temperature and stirred for 60 min. The resulting solution was added to a solution of Compound (8) in DIPEA/DCM solution over 2 h maintaining 15° C. After the addition, the reaction mixture was stirred for 2 h at the same temperature and then cooled down to 5° C. The reaction was quenched while maintaining the temperature below 10° C. by adding 15% w/w citric acid aqueous solution (250 mL) until pH 4-5. After separating the two layers, the organic layer was concentrated under reduced pressure and the aqueous layer was extracted with DIPE (320 mL). The combined organic layers were washed with water (3×90 mL) and with 25% w/w NaCl aq. solution (90 mL). The organic layer was concentrated under reduced pressure to yield 30.2 g of (9) as a yellow oil (95% yield).

Characterization data of Tert-butyl(S)-3-methyl-4-oxo-2-oxa-8-azaspiro[4.5]decane-8-carboxylate (9). Direct-phase chiral HPLC (column: AY-H Chiralpak, 150 mm*4.6 mm, 5 μm, column temperature: 40° C., UV: 210 nm, flow rate: 2 mL/min, injection volume: 20 μL, sample concentration: 5 mg/mL, diluent: isopropyl alcohol, mobile phase (A: n-hexane, B: isopropyl alcohol, 10% B)) of a 99.7:0.3 enantiomeric mixture gave two peaks at rt 4.427 min (99.71%) and 5.871 min (0.29%).

### Example 8. (S)-3-methyl-2-oxa-8-azaspiro[4.5]decan-4-one hydrochloride (10)⋅HCl

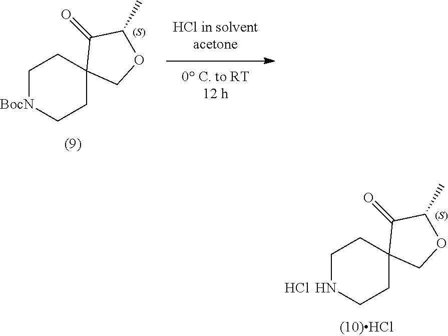

Condition 1. To a solution of tert-butyl(S)-3-methyl-4-oxo-2-oxa-8-azaspiro[4.5]decane-8-carboxylate (20.0 g, 74.3 mmol) in acetone (200 mL) at 0° C. was added 4 M HCl in dioxane (46.5 mL, 185.8 mmol) and the mixture was left to reach RT and stirred further for 12 h before collecting the resulting solid by filtration. The product(S)-3-methyl-2-oxa-8-azaspiro[4.5]decan-4-one hydrochloride (10). HCl was obtained as a white solid (13.0 g, 85% yield).

Condition 2. To a solution of tert-butyl(S)-3-methyl-4-oxo-2-oxa-8-azaspiro[4.5]decane-8-carboxylate (16.0 g, 59.4 mmol) in acetone (200 mL) at 0° C. was added 12 M HCl in water (9.9 mL, 118.8 mmol) and the mixture was left to reach RT and stirred further for 12 h before collecting the resulting solid by filtration. The product(S)-3-methyl-2-oxa-8-azaspiro[4.5]decan-4-one hydrochloride (10)⋅HCl was obtained as a white solid (9.8 g, 80% yield).

Condition 3. To a solution of tert-butyl(S)-3-methyl-4-oxo-2-oxa-8-azaspiro[4.5]decane-8-carboxylate (20.0 g, 74.3 mmol) in acetone (120 mL) at 0° C., HCl 13% w/w in EtOH (45.9 g, 163.4 mmol) was added and the mixture was left to reach RT and stirred further for 12 h before collecting the resulting solid by filtration. The product(S)-3-methyl-2-oxa-8-azaspiro[4.5]decan-4-one hydrochloride was obtained as a white solid (12.7 g, 83% yield).

Condition 4. To a solution of tert-butyl(S)-3-methyl-4-oxo-2-oxa-8-azaspiro[4.5]decane-8-carboxylate (16.0 g, 59.4 mmol) in acetone (65 mL) at 0° C., HCl 15% w/w in MeOH (31.8 g, 130.7 mmol) was added and the mixture was left to reach RT and stirred further for 12 h before collecting the resulting solid by filtration. The product(S)-3-methyl-2-oxa-8-azaspiro[4.5]decan-4-one hydrochloride was obtained as white solid (9.8 g, 80% yield).

Condition 5. To a solution of tert-butyl(S)-3-methyl-4-oxo-2-oxa-8-azaspiro[4.5]decane-8-carboxylate (16.0 g, 59.4 mmol) in acetone (80 mL) at 0° C., HCl 10% w/w in i-PrOH (47.7 g, 130.7 mmol) was added and the mixture was left to reach RT and stirred further for 12 h before collecting the resulting solid by filtration. The product(S)-3-methyl-2-oxa-8-azaspiro[4.5]decan-4-one hydrochloride was obtained as white solid (10.6 g, 87% yield).

Characterization data of(S)-3-methyl-2-oxa-8-azaspiro[4.5]decan-4-one hydrochloride (10)⋅HCl. GC after free-base releasing and extraction into organic phase (DCM) (column: RTX-5 Amine, 30 m×0.53 μm, 3 μm, gradient temperature: hold for 3 min at 100° C., rate at 10° C./min to 220° C. and hold for 5 min, rate at 20° C./min to 280° C. hold for 10 minutes, flow rate: 3.0 mL/min, injector temperature: 280° C., injection volume: 1 μL, split ratio: 1-10, FID detector temperature: 280° C., FID air flow rate: 400 mL/min, FID H₂ flow rate: 40 mL/min, FID Make up flow rate: 25 mL/min) gave a peak at rt 10.061 min (99.44%). Direct-phase chiral HPLC after derivatization with benzoyl chloride (column: AY-H Chiralpak, 150 mm*4.6 mm, 5 μm, column temperature: 40° C., UV: 205 nm, flow rate: 2 mL/min, injection volume: 10 μL, sample concentration: 1 mg/mL, mobile phase (A: n-hexane, B: ethanol, 20% B)) of a 99.1:0.9 enantiomeric mixture gave two peaks at rt 6.392 min (0.92%) and 7.232 min (99.08%).

Examples 9-18 provide details for converting Compound (10), or a salt thereof, to Compound (11) according to the general reaction scheme shown below. Details are also provided in PCT Application No. PCT/US2021/064040, the disclosure of which is incorporated herein by reference.

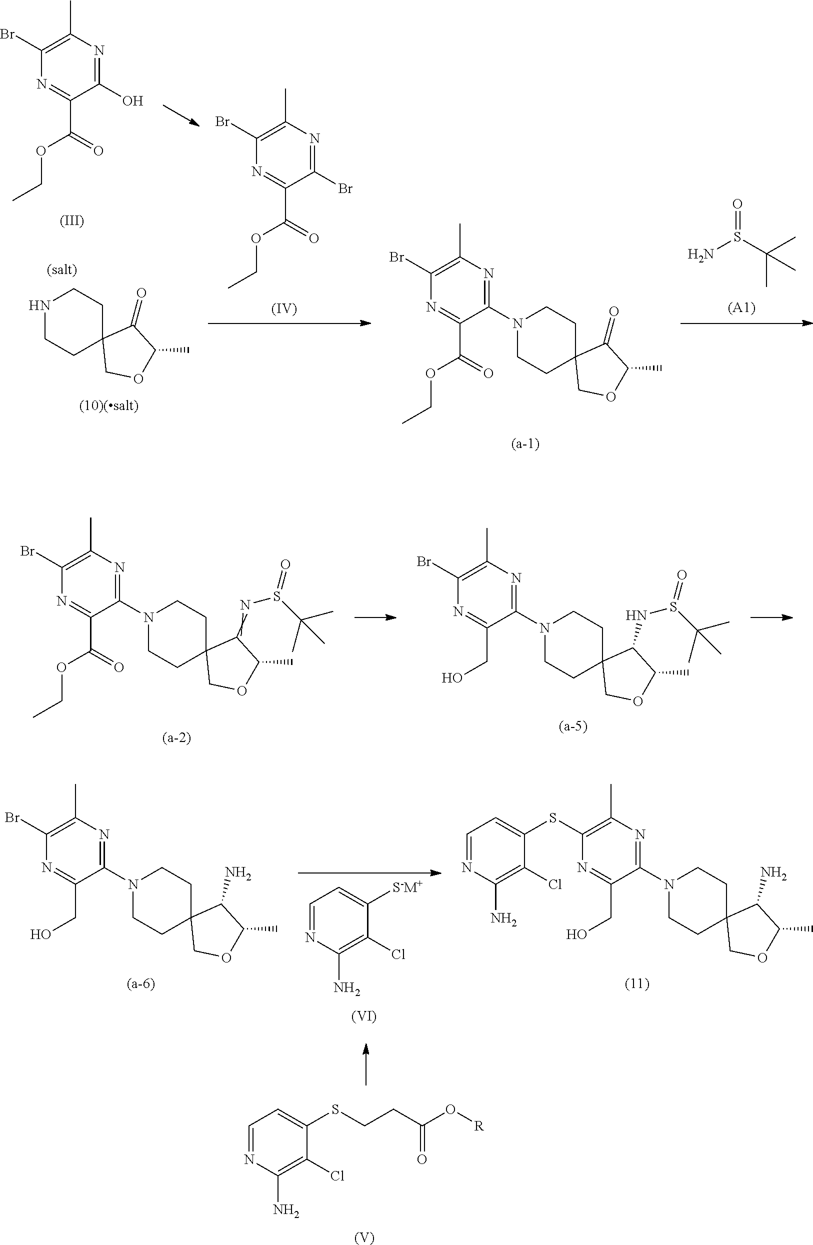

### Example 9. Preparation of Ethyl(S)-6-bromo-5-methyl-3-(3-methyl-4-oxo-2-oxa-8-azaspiro[4.5]decan-8-yl) pyrazine-2-carboxylate (Compound (a-1))

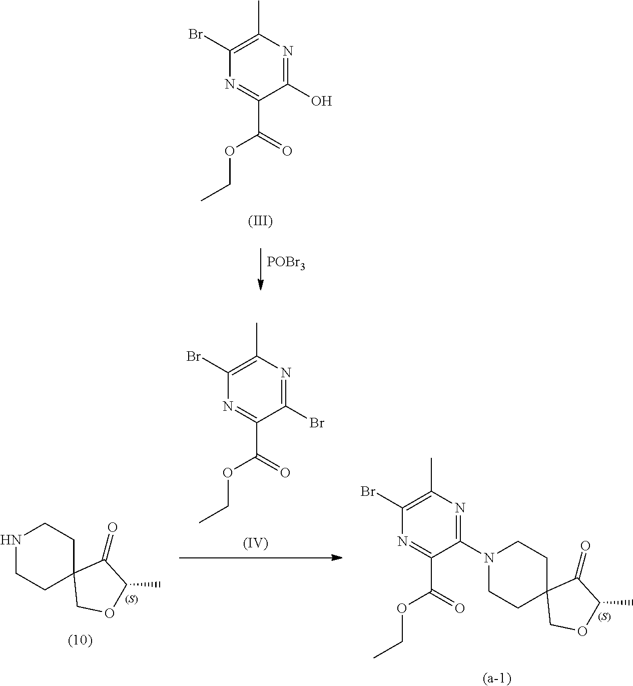

Compound (a-1) was prepared by the series of reactions shown in the scheme above. Briefly, Compound (IV) was prepared by reaction of Compound (III) with POBr₃. Next, Compound (10), or a salt thereof, was coupled to Compound (IV) to give Compound (a-1). Synthesis of Compound (III).

Route A. Compound (III) can be prepared according to the procedures described in U.S. Pat. No. 10,590,090 as shown in the scheme below.

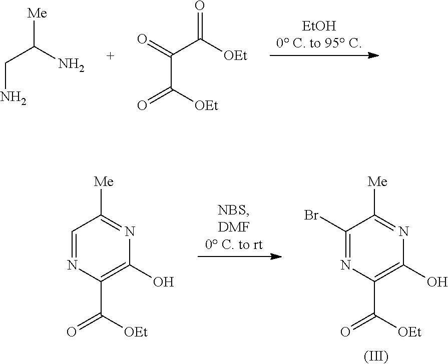

Route B. Compound (III) was prepared according to the scheme below. In this reaction, hydrated ketomalonate replaces the ketomalonate of Route A above.

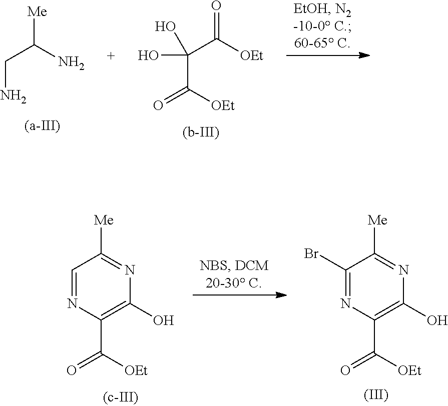

Step 1. Synthesis of (c-III).

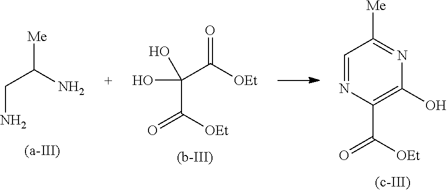 [SMILES: CC(N)CN ; CCOC(=O)C(O)(O)C(=O)OCC ; CCOC(=O)c1ncc(C)nc1O]

To a 1500-L reactor was charged EtOH (810 kg, 5 vol). The reactor was evacuated and backfilled with nitrogen atmosphere two times. To the reactor was charged propane-1,2-diamine (a-III) (68.4 kg, 922.8 mol, 1.05 equiv). The resulting mixture was cooled to −8 to −15° C. To the reactor was then charged diethyl 2,2-dihydroxymalonate (b-III) (168.9 kg, 878.9 mol, 1.0 equiv) in fifteen equal portions over 4 hours, maintaining a temperature of −10 to 0° C. The reaction mixture was maintained at −10 to 0° C. for 2 hours during which time a white solid precipitated and at which point GC monitoring showed first reaction completion. The reaction mixture was warmed to 60-65° C. during which time a clear solution formed. The reaction mixture was maintained at 60-65° C. for 15 hours at which point HPLC monitoring showed second reaction completion.

The reaction was cooled to 25-35° C. and then distilled to ˜1.2 vol, maintaining temperature below 45° C. The concentrate was cooled to 0-5° C. over 1 hour and then maintained at 0-5° C. for 1 hour. To this mixture was charged methyl tert-butyl ether (MTBE) (63.0 kg, 0.5 vol) over 1 hour, maintaining a temperature of 0-5° C. The resulting mixture was maintained at 0-5° C. for 1 hour and then filtered, washing the cake with EtOH/MTBE (2×1:1 (v/v), 68.0 kg). The cake was dried under reduced pressure at 40-45° C. for 13 hours to afford ethyl 3-hydroxy-5-methylpyrazine-2-carboxylate (c-III) as a brick-red solid (32.9 kg, 99.1% a/a HPLC purity, 96.9% w/w qNMR assay, 21% assay-corrected yield).

¹H NMR (400 MHZ, DMSO-d₆) δ 12.74 (br s, 1H), 7.36 (s, 1H), 4.26 (q, J=7 Hz, 2H), 2.24 (s, 3H), 1.26 (t, J=6 Hz, 3H).

### Step 2. Synthesis of Compound (III).

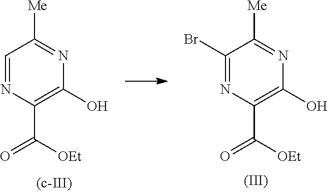

To a 1000-L reactor was charged ethyl 3-hydroxy-5-methylpyrazine-2-carboxylate (c-III) (32.9 kg, 96.9% w/w assay, 175.0 mol, 1.0 equiv) and DCM (450 kg, 10 vol). The reactor was evacuated and backfilled with nitrogen atmosphere two times. To the reactor was charged NBS (32.1 kg, 180.4 mol, 1.03 equiv) in five equal portions over 2.5 hours, maintaining a temperature of 20-30° C. The reaction mixture was maintained at 20-30° C. for 1 hour at which point was charged more NBS (400 g, 2.2 mol, 0.01 equiv), maintaining a temperature of 20-30° C. The reaction mixture was maintained at 20-30° C. for 0.5 hours as which point HPLC monitoring showed reaction completion.

To the reaction was charged 5% w/w aqueous NaHSO₃ (160 kg, 5 vol). The layers were separated. The organic layer was washed with water (160 kg, 5 vol), brine (160 kg, 5 vol), and then filtered. The filtrate was concentrated to ˜ 2 vol (˜70 L). To the concentrate was added MTBE (45 kg, 2.0 vol). This was concentrated to ˜ 2 vol (˜70 L). To the concentrate was added n-heptane (112 kg, 5 vol) over 1.5 hours, maintaining a temperature of 10-20° C. The resulting suspension was maintained at 10-20° C. for 1 hour and then filtered. The cake was dried at 40-45° C. for 5 hours to afford ethyl 6-bromo-3-hydroxy-5-methylpyrazine- 2-carboxylate (III) as a light-orange solid (37.8 kg, 99.6% a/a HPLC purity, 81% assay-corrected yield).

¹H NMR (400 MHZ, CDCl₃) δ 11.24 (br s, 1H), 4.51 (q, J=7 Hz, 2H), 2.66 (s, 3H), 1.44 (t, J=6 Hz, 3H).

MS (ESI+): calculated, 260.00; found, 260.7.

### Synthesis of Compound (IV).

Compound (IV) was prepared according to the scheme below.

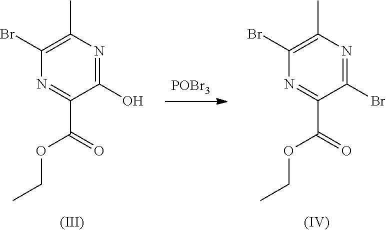

Synthesis A. Compound (III) (60 g, 1 equiv.) was treated with POBr₃ (85.7 g, 1.3 equiv.) and DMF (2.7 g, 0.16 equiv.) in DCM (450 mL) at 20-30° C. for 90 h. To the reaction mixture was added K₂CO₃ (1320 g, 10% solution) and the organic layer was washed with water. The solvent was removed under reduced pressure to yield Compound (IV) (75.6 g, 94% yield).

Synthesis B. To a reactor was charged DCM (458 kg), POBr₃ (58.0 kg, 1.3 equiv), and DMF (2.2. kg, 0.2 equiv). The mixture was maintained at 20-30° C. for 1 hour. To the reactor was then charged ethyl 6-bromo-3-hydroxy-5-methylpyrazine-2-carboxylate (III) (40.0 kg, 1 equiv) and additional DCM (22 kg). The reaction was heated to 30-40° C. and maintained at that temperature for 60 hours at which point UPLC monitoring showed reaction completion.

The reaction was then cooled to 20-30° C. To the reactor was charged 10% w/w aq. K₂CO₃ (851 kg), maintaining a temperature below 30° C. The layers were separated and the aqueous layer extracted with DCM (280 kg). The combined organic layers were washed with water (2×220 kg) and then concentrated to ˜4 vol under reduced pressure at ≤35° C. to afford ethyl 3,6-dibromo-5-methylpyrazine-2-carboxylate (IV) as a DCM solution (160.4 kg, 98.3% a/a UPLC purity, 30.0% w/w assay, 97% assay-corrected yield).

Synthesis of Compound (a-1).

Compound (a-1) was prepared according to the scheme below.

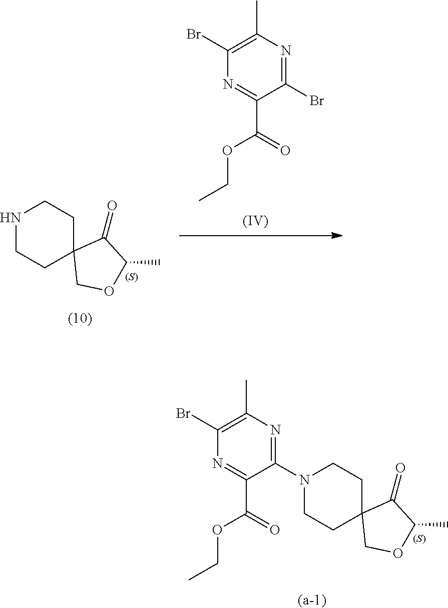

Compound (10) (200 g, 1 equiv.) was treated with Compound (IV) (135 g, 1.05 equiv.) and triethylamine (156.2 g, 2.5 equiv.) in acetone (1400 mL). After stirring for 20 h. at 20-30° C., acetic acid (18.5 g, 0.5 equiv.) was added at 15-25° C. Next, water (1400 mL) was slowly added and the reaction mixture was stirred for 1.5 h. at 20-30° C. The resulting solid was isolated by filtration and washed with water/acetone (1:1 v/v) to afford Compound (a-1) (236.5 g, 91.3% yield) with chiral purity >99%.

### Example 10. Preparation of Ethyl 6-bromo-3-[(3S)-4-[(S)-tert-butylsulfinyl]imino-3-methyl-2-oxa-8-azaspiro[4.5]decan-8-yl]-5-methyl-pyrazine-2-carboxylate (Sulfinyl Imine Compound (a-2b))

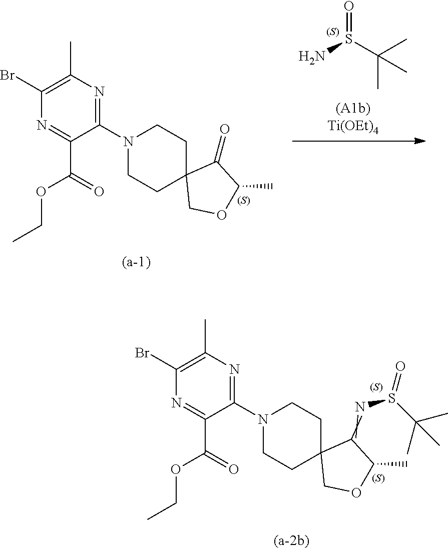

As described in detail below and in Example 11, the sulfinyl imine Compounds (a-2b) and (a-2a) were prepared from Compound (a-1) and(S)-sulfinamide (A1b) or (R)-sulfinamide (A1a), respectively. In each of the methods studied, the expected product (“EP” or “Compound (a-2a)” or “Compound (a-2b)”) was formed in addition to the corresponding isopropyl ester. As shown in Example 14, the presence of the isopropyl ester did not affect the synthesis of subsequent molecules as both esters (ethyl and isopropyl) were ultimately reduced to the corresponding alcohol. As such, it is not necessary to purify the product containing Compound (a-2a) or Compound (a-2b) to remove the corresponding isopropyl ester.

Methods 1-3 (small scale tests). Three small scale syntheses were conducted to determine the optimal amount of titanium reagent to be used. As summarized below in Table 1, no major drawbacks appeared in terms of conversion or impurity profile, and 2 equivalents of Ti(OEt)₄ was selected for scale-up.

TABLE 1

| Method | | | |
|---|---|---|---|
| No. | Scale | Conditions | Results |
| 1 | 500 mg | Compound (a-1): 1 eq. | 437.4 mg isolated (mixture |
| | | MeTHF: 10 vol. | of EP and isopropyl ester) |
| | | Ti(OEt)₄: 4 eq. | LCMS: |
| | | S-sulfinamide (A1b): | EP - 2 peaks 81.4% + |
| | | 1.1 eq reflux | 2.75% |
| | | 2 hr: 59% conversion | Isopropyl ester - 12.81% |
| | | 21 hr: 90.6% conversion | |
| | | 24 hr: 91.6% conversion | |
| 2 | 500 mg | Compound (a-1): 1 eq. | 464 mg isolated (mixture |
| | | MeTHF: 10 vol. | of EP and isopropyl ester) |
| | | Ti(OEt)₄: 3 eq. | LCMS: |
| | | S-sulfinamide (A1b): | EP - 2 peaks 83.2% + |
| | | 1.1 eq reflux | 2.53% |
| | | 2 hr: 56.3% conversion | Isopropyl ester - 12.25% |
| | | 21 hr: 90.2% conversion | |
| | | 24 hr: 93.6% conversion | |
| 3 | 500 mg | Compound (a-1): 1 eq. | 472 mg isolated (mixture |
| | | MeTHF: 10 vol. | of EP and isopropyl ester) |
| | | Ti(OEt)₄: 2 eq. | LCMS: |
| | | S-sulfinamide (A1b): | EP - 2 peaks 84.12% + |
| | | 1.1 eq reflux | 2.53% |
| | | 2 hr: 52.5% conversion | Isopropyl ester -11.215% |
| | | 21 hr: 86.8% conversion | |
| | | 24 hr: 89.4% conversion | |

Method 4 (large scale). In a 10 mL vial at room temperature was introduced, under N₂, ethyl(S)-6-bromo-5-methyl-3-(3-methyl-4-oxo-2-oxa-8-azaspiro[4.5]decan-8-yl) pyrazine-2-carboxylate (Compound (a-1)) (500 mg, 1.21 mmol) and anhydrous 2-methyltetrahydrofuran (2 mL). To the yellow solution was added dropwise, over 2 min, a solution of Ti(OEt)₄ (96%, 530 μL, 2.43 mmol) in anhydrous 2-methyltetrahydrofuran (1.5 mL). To this solution was then added dropwise, over 2 min, a solution of 2-methylpropane-2-sulfinamide(S) (A1b) (162 mg, 1.33 mmol) in anhydrous 2-methyltetrahydrofuran (1.5 mL). The resulting yellow solution was then heated up to 80° C. for 24 h until conversion reached 89%. The reaction mixture was then cooled down to 0-5° C. using an ice bath.

To the cooled reaction mixture was added an aqueous citric acid solution (2.5 mL, 1 M, 5 vol.). To the thick suspension was added 2-methyltetrahydrofuran (2.5 mL, 5 vol.). The reaction mixture turned to a thin suspension that was filtered. The solid was washed 4 times with 2-methyltetrahydrofuran (2.5 mL, 4×5 vol.) to recover all EP. The layers were separated, and the organic layer was washed twice with an aqueous citric acid solution (1 M, 5 mL, 2×10 vol.) and four times with water (5 mL, 4×10 vol.). Alternatively, the work-up procedure was performed using water in place of the aqueous citric acid solution as described in Example 3.

The organic layer was concentrated to dryness to afford crude material (655 mg), which was purified by flash chromatography to obtain the expected product (“EP” or Compound (a-2b)) as a yellow oil in a mixture of ethyl and isopropyl ester in a ratio 85/15 (472 mg, 63% yield (ethyl ester)).

The product was analyzed using LCMS (UPLC Column Acquity UPLC CSH C18, 2.1×50 mm, 1.7 μm; eluant A=H₂O+0.02% HCOOH; eluent B=CH₃CN+0.02% HCOOH; oven temp=55° C.; gradient: t0 2% B, t4.5 min 98% B, t5 min 2% B; flow rate=1 mL/min; electrospray ionization mode-capillary, 3 kV sample cone 15/30V). LCMS analysis showed peaks at 514 (ethyl ester) and 528 (isopropyl ester).

### Example 11. Preparation of Ethyl 6-bromo-3-[(3S)-4-[(R)-tert-butylsulfinyl]imino-3-methyl-2-oxa-8-azaspiro[4.5]decan-8-yl]-5-methyl-pyrazine-2-carboxylate (Sulfinyl Imine Compound (a-2a))

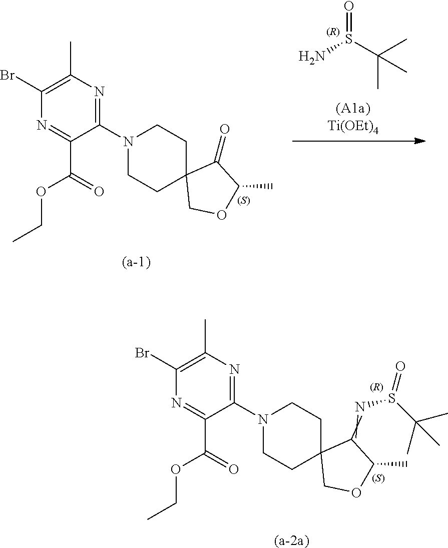

Small scale synthesis. The sulfinyl imine Compound (a-2a) was prepared from the (R)-sulfinamide (A1a) following the same protocol as used for the(S)-sulfinamide in Example 10. The retention times of the (R) (Compound (a-2a)) and(S) (Compound (a-2b)) diastereoisomers were similar, with the (R)-sulfinamide analog (Compound (a-2a)) being slightly less polar.

Large scale synthesis. In a 250 mL round-bottom flask at room temperature was introduced, under N₂, ethyl(S)-6-bromo-5-methyl-3-(3-methyl-4-oxo-2-oxa-8-azaspiro[4.5]decan-8-yl) pyrazine-2-carboxylate (Compound (a-1)) (5 g, 12.1 mmol) and anhydrous 2-methyltetrahydrofuran (25 mL). To this yellow solution was added dropwise, over 15 min, a solution of 2-methylpropane-2-sulfinamide (R) (A1a) (2.94 g, 24.3 mmol, 2 eq.) in 2-anhydrous methyltetrahydrofuran (12.5 mL). To the yellow solution was added dropwise, over 20 min, a solution of Ti(OEt)₄ (99.99%, 4.6 mL, 21.8 mmol, 1.8 eq.) in anhydrous 2-methyltetrahydrofuran (12.5 mL). The resulting clear yellow solution was then heated at 80° C. for 15 h. and complete conversion was observed. The reaction mixture was then cooled down to room temperature. To the reaction mixture was added an aqueous citric acid solution (30 mL, 1 M). The reaction mixture turned to a thin suspension that was filtered on cardboard. The solid was washed 4 times with 2-methyltetrahydrofuran (20 mL, 4×4 vol.). The layers were separated, and the organic layer was washed twice with an aqueous citric acid solution (30 mL, 1 M, 2×6 vol.) and four times with water (30 mL, 4×6 vol.). The organic layer was concentrated to dryness to afford crude product containing Compound (a-2a) (6.24 g) and about 10-20% of the corresponding isopropyl ester. Alternatively, the work-up procedure was performed using water in place of the aqueous citric acid solution. Briefly, once the reaction was complete, the mixture was cooled to 20-25° C. and water (8 equiv. relative to Ti(OEt)+) was added to the orange solution. The suspension was filtered over a pad of Celite® and the cake was washed with MeTHF (4*5 vol). The yellow filtrate was kept as a solution of MeTHF containing Compound (a-2a) and the corresponding isopropyl ester.

The crude product was analyzed using the same LCMS conditions described in Example 10 for Compound (a-2b). LCMS analysis showed a peak at 514 (ethyl ester) and an impurity peak at 528 (isopropyl ester). ¹H NMR (DMSO-d₆, 500 MHZ): δ (ppm) 4.88 (q, J=6.4 Hz, 1H), 4.31 (q, J=7.1 Hz, 2H), 3.98 (q, J=9.5 Hz, 2H), 3.68-3.90 (m, 2H), 3.02-3.21 (m, 2H), 2.48 (s, 3H), 1.56-1.87 (m, 4H), 1.42 (d, J=6.8 Hz, 3H), 1.27-1.33 (m, 3H), 1.17 (s, 9H).

### Example 12. Preparation of (R/S)—N-[(3S,4S)-8-[5-bromo-3-(hydroxymethyl)-6-methyl-pyrazin-2-yl]-3-methyl-2-oxa-8-azaspiro[4.5]decan-4-yl]-2-methyl-propane-2-sulfinamide (Compound (a-3a) and Compound (a-3b′))

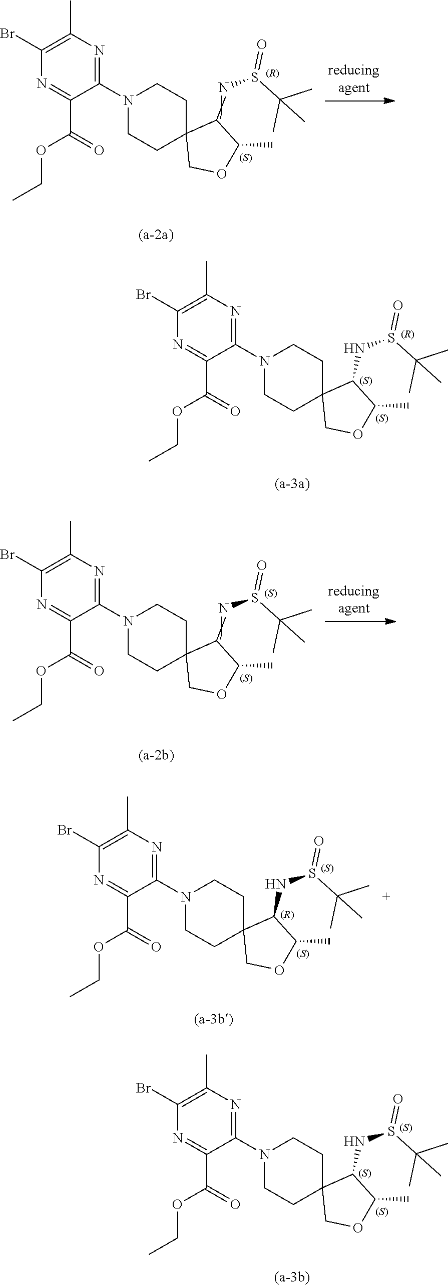

Methods 1-8—reducing the (R)-sulfinyl imine (Compound (a-2a)), The reduction of the (R)-sulfinyl imine (Compound (a-2a)) was examined using four different reducing agents under varying conditions (Table 2). Methods 1˜4 varied the reducing agent, and methods 5-8 examined the effect of temperature on reductions with DIBAL-H.

TABLE 2

| Method No. | Scale | Conditions | Results |
|---|---|---|---|
| 1 | 100 mg | 1 eq. R-sulfinyl imine | Expected MW observed |
| | | MeTHF (10 vol.) | but no EP (reduction |
| | | L-selectride (1M, | occurred but product |
| | | 1.5 eq) −78° C. | does not have desired |
| | | 2 hr: 75% conversion | stereochemistry; |
| | | | obtained 3S,4R isomer) |
| 2 | 100 mg | 1 eq. R-sulfinyl imine | Degradation after |
| | | MeTHF (10 vol.) | additional 1.5 eq. |
| | | LiBHEt₃ (1M, | LiBHEt₃ |
| | | 2*1.5 eq) −78° C. | |
| | | 2 hr: MW = 517 and | |
| | | degradation products | |
| 3 | 100 mg | 1 eq. R-sulfinyl imine | MW observed after |
| | | MeTHF (10 vol.) | warming at 20-25° C., |
| | | NaBH₄ | no complete conversion |
| | | (2*4 eq) −78° C. | (84%), no degradation |
| | | 2 hr: no reaction at −78° C. | after treatment |
| 4 | 172 mg | 1 eq. R-sulfinyl imine | 141 mg isolated (88% |
| | | MeTHF (10 vol.) | LCMS) after FCC; mixture |
| | | DIBAL-H (1M, 1.5 eq) −78° C. | between EP (Compound |
| | | 2 hr: complete conversion | (3a)) and isopropyl |
| | | | ester, yield 80% |
| 5 | 100 mg | 1 eq. R-sulfinyl imine | Clean profile |
| | | MeTHF (10 vol.) | |
| | | DIBAL-H (1M, 1.5 eq) −50° C. | |
| | | 1.5 hr: complete conversion | |
| 6 | 100 mg | 1 eq. R-sulfinyl imine | Clean profile |
| | | MeTHF (10 vol.) | |
| | | DIBAL-H (1M, 1.5 eq) −20° C. | |
| | | 1.5 hr: complete conversion | |
| 7 | 100 mg | 1 eq. R-sulfinyl imine | Clean profile |
| | | MeTHF (10 vol.) | |
| | | DIBAL-H (1M, 1.5 eq) | |
| | | 0° C. | |
| | | 1.5 hr: complete conversion | |
| 8 | 100 mg | 1 eq. R-sulfinyl imine | Complete conversion but |
| | | MeTHF (10 vol) | some impurities observed |
| | | DIBAL-H (1M, 1.5 eq) | |
| | | Room temperature | |
| | | 1.5 hr: complete conversion | |

Methods 9-13—reducing the(S)-sulfinyl imine (Compound (a-2b)). The reduction of the(S)-sulfinyl imine (Compound (a-2b)) was examined using four different reducing agents (Table 3).

TABLE 3

| Method No. | Scale | Conditions | Results |
|---|---|---|---|
| 9 | 200 mg | 1 eq. S-sulfinyl imine | MW observed but not same |
| | | MeTHF (10 vol.) | retention time; reduction of |
| | | L-selectride (1M, | pyrimidine suspected |
| | | 2*1.5 eq), −78° C. | because of different rt; |
| | | 2 hr: 50% conversion | after purification, complete |
| | | | degradation to MW = 353 |
| | | | without Br, unidentified |
| | | | compound |
| 10 | 200 mg | 1 eq. S-sulfinyl imine | Several peaks with expected |
| | | MeTHF (10 vol.) | MW. Degradation also |
| | | LiBHEt₃ (1M, | observed. Attempt to purify |
| | | 2*1.5 eq), −78° C., 2 hr | by FCC not successful |
| 11 | 200 mg | 1 eq. S-sulfinyl imine | No reaction at −78° C. |
| | | MeTHF (10 vol.) | Reduction seems to occur at |
| | | NaBH₄ | higher temp. After 5 days at |
| | | (2*4 eq) −78° C., 2 hr | rt, reduction of ester moiety |
| 13 | 200 mg | 1 eq. S-sulfinyl imine | 112.7 mg isolated after FCC; |
| | | MeTHF (10 vol.) | mixture between EP |
| | | DIBAL-H (1M, | (Compound (a-3b′)) and |
| | | 2*1.5 eq) −78° C. | EP-isopropyl; corrected |
| | | 2 hr: 32.9% conversion | yield = 58% |

Compound (a-3b), EP (ethyl ester), 3S(Me), 4S(NH): Rt1=3.31 min (9.1%); Compound (a-3b′), EP (ethyl ester), 3S(Me), 4R(NH): Rt2-3.40 min (66.2%); Compound (a-3b′), isopropyl ester, 3S(Me), 4R(NH): Rt3=3.59 min (9.1%); Compound (a-3b) isopropyl ester, 3S(Me), 4S(NH): Rt6=3.5 min (1.1%).

Method 14—reducing the (R)-sulfinyl imine with DIBAL-H (scale-up). In a 3-neck round bottom flask at room temperature was introduced, under N₂ atmosphere, ethyl 6-bromo-3-[(3S,4Z)-4-[(R)-tert-butylsulfinyl]imino-3-methyl-2-oxa-8-azaspiro[4.5]decan-8-yl]-5-methyl-pyrazine-2-carboxylate (Compound (a-2a)) (1.0091 g, 1.4 mmol) and MeTHF (10 mL). The reaction mixture was cooled down to −78° C. and then DIBAL-H (2.1 mL, 1 M in THF) was added. The reaction mixture was stirred at −78° C. for 1 h and quenched with Rochelle salt. The organic layer was separated and concentrated to dryness before flash chromatography purification (CH₂Cl₂/acetone). Compound (a-3a) was isolated as a yellow oil (571 mg, 79%). LCMS analysis showed a peak at 516. ¹H NMR (500 MHz, DMSO-d₆, 300K) 8 ppm 5.10 (d, J=10.8 Hz, 1H), 4.32 (q, J=7.1 Hz, 2H), 4.13 (t, J=6.2 Hz, 1H), 3.82 (d, J=8.8 Hz, 1H), 3.73 (br d, J=13.7 Hz, 1H), 3.60 (br d, J=13.7 Hz, 1H), 3.50 (d, J=8.8 Hz, 1H), 3.41 (dd, J=10.8, 6.1 Hz, 1H), 3.03-3.21 (m, 2H), 2.48 (s, 3H), 1.69-1.85 (m, 2H), 1.49-1.64 (m, 2H), 1.31 (t, J=7.2 Hz, 3H), 1.15 (s, 9H), 1.09 (d, J=6.4 Hz, 3H).

### Example 13. Preparation of Amino Compound (a-4)

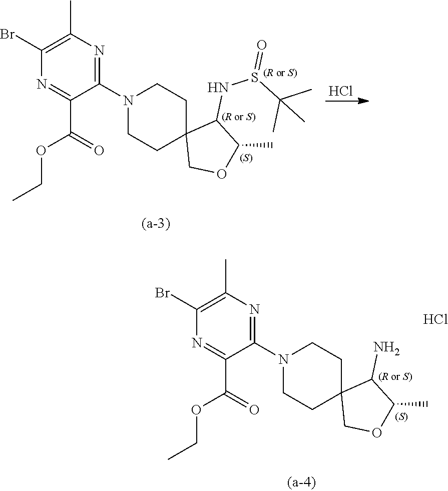

In a 3-neck round-bottom flask at room temperature was introduced ethyl 6-bromo-3-[(3S,4S)-4-[[(R)-tert-butylsulfinyl]amino]-3-methyl-2-oxa-8-azaspiro[4.5]decan-8-yl]-5-methyl-pyrazine-2-carboxylate (Compound (a-3a)) (92%, 430 mg, 0.76 mmol, 1 eq.) and EtOH (3.7 mL). The reaction mixture was cooled down to 0-5° C., and then 0.87 mL HCl (1.25 M EtOH, 1.09 mmol, 1.5 eq.) was added. The reaction mixture was brought back to room temperature and stirred for 1 hr before being directly concentrated to dryness to afford the amine Compound (a-4) (S form) (381 mg, 94% yield (crude product)). LCMS analysis showed a peak at 412. ¹H NMR (600 MHz, DMSO-d₆) δ ppm 7.60-8.24 (m, 3H) 4.29-4.36 (m, 2H) 4.15-4.23 (m, 1H) 3.83-3.87 (m, 1H) 3.58-3.80 (m, 3H) 3.39-3.43 (m, 1H) 2.97-3.16 (m, 2H) 2.49 (s, 3H) 1.52-1.83 (m, 4H) 1.29-1.34 (m, 3H) 1.18-1.22 (m, 3H).

### Example 14. Preparation of Compound (a-5a)

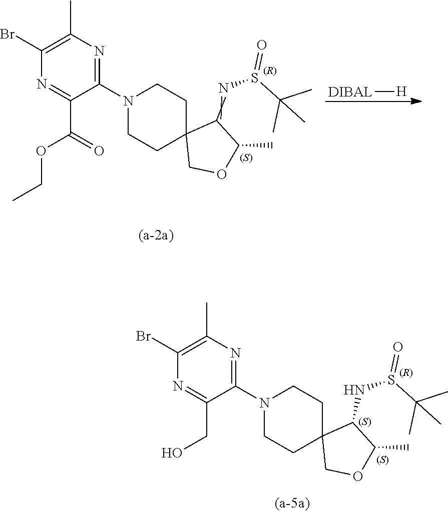

As discussed above in Example 10, the sulfinyl imine Compounds (a-2b) and (a-2a) were prepared from ethyl(S)-6-bromo-5-methyl-3-(3-methyl-4-oxo-2-oxa-8-azaspiro[4.5]decan-8-yl) pyrazine-2-carboxylate (Compound (a-1)) and(S)-sulfinamide (A1b) or (R)-sulfinamide (A1a), respectively. In each of the methods studied, the expected product (“EP” or “Compound (a-2a)” or “Compound (a-2b)”) was formed in addition to the corresponding isopropyl ester. In order to demonstrate that both esters (ethyl and isopropyl) could be reduced together in a mixture to the corresponding alcohol, a small amount of the mixed product of Compound (a-2a) was purified into the ethyl ester and isopropyl ester fractions by chromatography. Next, the reduction reaction was run on all three products: (1) the isolated isopropyl ester; (2) the isolated ethyl ester; and (3) the mixture of esters (ethyl and isopropyl). As shown below, the presence of the isopropyl ester did not affect the reduction to the corresponding alcohol.

Method 1—Reduction of isopropyl ester. In a 20 mL vial under N₂ atmosphere was introduced isopropyl 6-bromo-3-[(3S,4Z)-4-[(R)-tert-butylsulfinyl]imino-3-methyl-2-oxa-8-azaspiro[4.5]decan-8-yl]-5-methyl-pyrazine-2-carboxylate (465 mg, 0.879 mmol) and anhydrous 2-methyltetrahydrofuran (5 mL). To the yellow solution, cooled down to −20° C., was added DIBAL-H (1.5 mL, 1 M THF, 1.5 mmol). The reaction mixture was stirred at −20° C. for 1.5 hr. LCMS monitoring showed full conversion of the sulfinyl-imine moiety. Three additional amounts of DIBAL-H (3×1.5 mmol) were introduced at −20° C. every 1 h 20 min to provide complete conversion of the isopropyl ester into the corresponding alcohol.

Once the conversion was complete, to the yellow reaction mixture was added Rochelle salt (10 mL, 20 vol.). The reaction mixture was allowed to reach room temperature. The thick suspension was stirred overnight at room temperature. The layers were separated, and the organic layer was washed twice with Rochelle salt solution (10 mL, 20 vol.). The clear yellow organic layer was dried and concentrated to dryness to afford crude Compound (a-5a) (478 mg, 97% yield). LCMS analysis confirmed the expected product.

Method 2—Reduction of Compound (a-2a)/isopropyl ester mixture. In a 20 mL vial under N₂ atmosphere was introduced an ethyl ester and isopropyl ester mixture (1:1) (280 mg) and anhydrous 2-methyltetrahydrofuran (2.8 mL). To the yellow solution, cooled down to −20° C., was added DIBAL-H (0.81 mL, 1 M THF, 0.815 mmol). The reaction mixture was stirred at −20° C. for 1 h. LCMS monitoring showed full reduction of the sulfinyl imine moiety. Two additional amounts of DIBAL-H (2×0.815 mmol) were introduced at −20° C. every 1 h to provide complete conversion of the esters (ethyl ester and isopropyl ester) into the corresponding alcohol (Compound (a-5a)).

Once the conversion was complete, to the yellow reaction mixture was added Rochelle salt (2.8 mL, 10 vol.) and the reaction mixture was allowed to reach room temperature. The suspension was stirred overnight at room temperature. The layers were separated, and the organic layer was washed twice with Rochelle salt solution (2.8 mL, 2×10 vol.). The clear yellow organic layer was dried and concentrated to dryness to afford crude Compound (a-5a) (263 mg, 92% yield). LCMS analysis confirmed the expected product.

Method 3—Reduction of ethyl ester (Compound (a-2a)). In a 100 mL 4-neck round-bottom flask under N₂ atmosphere was introduced ethyl 6-bromo-3-[(3S,4Z)-4-[(R)-tert-butylsulfinyl]imino-3-methyl-2-oxa-8-azaspiro[4.5]decan-8-yl]-5-methyl-pyrazine-2-carboxylate (95%, 1.99 g, 3.66 mmol) and anhydrous 2-methyltetrahydrofuran (20 mL). To the yellow solution, cooled down to −20° C., was added DIBAL-H (5.8 mL, 1 M THF, 5.8 mmol) over 7 min. The reaction mixture was stirred at −20° C. for 1 h. LCMS monitoring showed complete reduction of the sulfinyl imine moiety. Five additional amounts of DIBAL-H (2×1.5 eq. and 3×1 eq.) were introduced at −20° C. every 1 h to provide complete conversion of the ethyl ester intermediate (Compound (a-3a)) into the corresponding alcohol (Compound (a-5a)).

Once the conversion was complete, the reaction mixture was heated up to 0-5° C., and to the yellow reaction mixture was added Rochelle salt solution (20 mL). The reaction mixture was allowed to reach room temperature. The thick suspension was stirred for 45 min. at room temperature. The layers were separated, and the aqueous layer was extracted twice with MeTHF (10 mL). The combined organic layers were washed twice with Rochelle salt solution (20 mL). The clear yellow organic layer was dried over Na₂SO₄, filtered, and concentrated to dryness to afford the crude expected alcohol (Compound (a-5a)) (1.6 g, 67% yield, LCMS purity 73%). LCMS analysis showed a peak at expected mass of 474. ¹H NMR (500 MHZ, DMSO-d₆, 300K) 8 ppm 5.45 (t, J=5.9 Hz, 1H), 5.09 (d, J=11.0 Hz, 1H), 4.42 (d, J=5.9 Hz, 2H), 4.12 (quin, J=6.4 Hz, 1H), 3.81 (d, J=8.6 Hz, 1H), 3.49 (d, J=8.8 Hz, 2H), 3.46-3.64 (m, 1H), 3.41 (dd, J=10.8, 6.1 Hz, 1H), 2.85-3.02 (m, 2H), 2.45 (s, 3H), 1.74-1.99 (m, 2H), 1.49-1.69 (m, 2H), 1.16 (s, 9H), 1.09 (d, J=6.6 Hz, 3H).

### Example 15. Preparation of Compound (a-6)

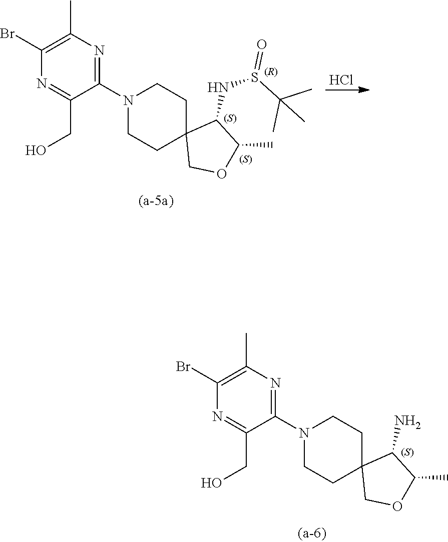

To a 1-neck round bottom flask was added Compound (a-5a) (113 mg, 0.23 mmol, 1 equiv.) and dry EtOH (1.5 mL). Next, HCl in EtOH (1.25 M, 0.27 mL, 0.34 mmol, 1.5 equiv.) was added at 0° C. The reaction mixture was placed under a nitrogen atmosphere and stirred at RT for 1 h, and then concentrated under vacuum to afford crude Compound (a-6) as an HCl salt (85.9 mg, LC-MS purity of 75%). ¹H NMR (600 MHZ, DMSO-d) 8 ppm 7.48-8.72 (m, 3H), 4.43 (s, 2H), 4.16-4.23 (m, 1H), 3.97-4.12 (m, 1H), 3.83-3.86 (m, 1H), 3.63-3.64 (m, 1H), 3.54-3.61 (m, 2H), 3.36-3.41 (m, 6H), 2.83-3.01 (m, 2H), 2.46 (s, 3H), 1.58-1.90 (m, 4H), 1.20-1.24 (m, 3H).

### Example 16. Preparation of Compound (a-6) (Scale-Up)

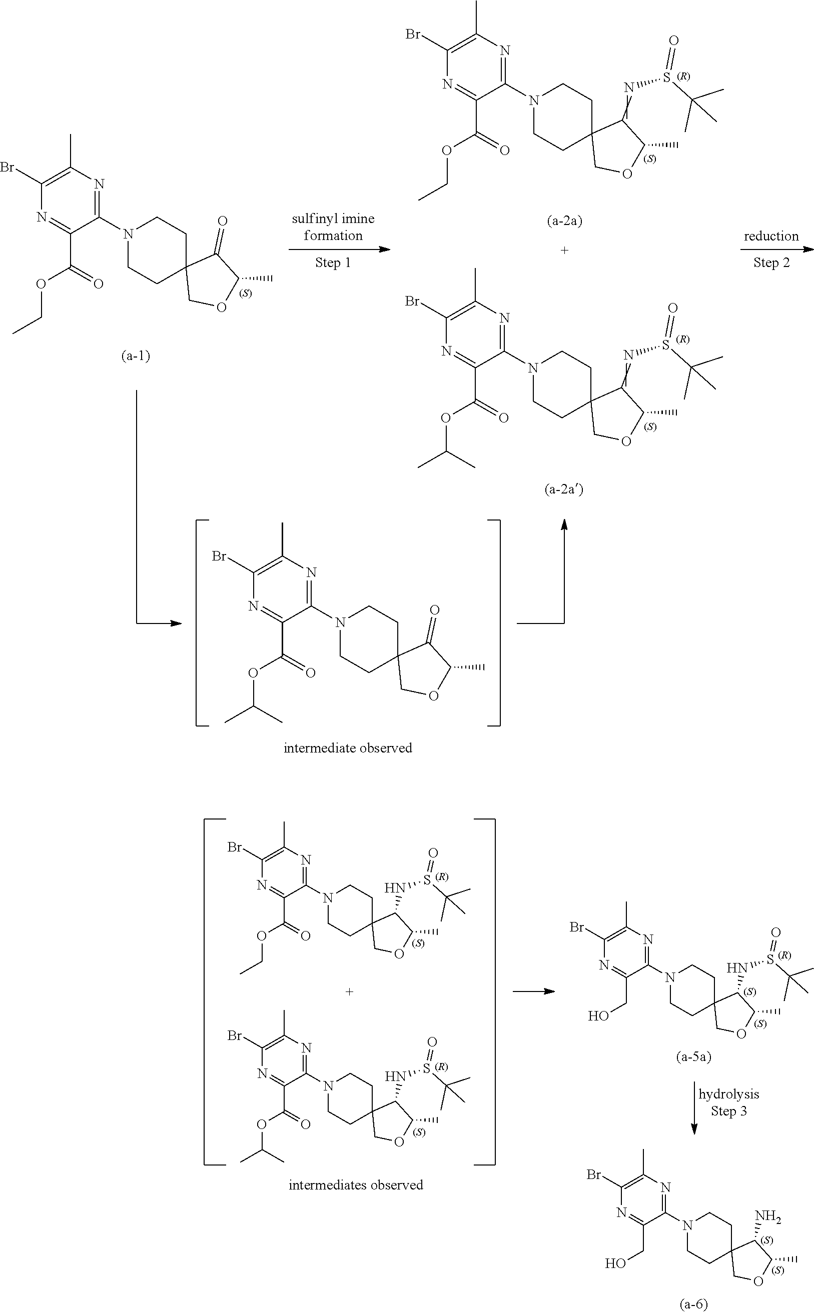

Step 1. Preparation of Compound (a-2a). To a 1 L, 4-neck, round-bottom flask equipped with thermometer, bleach trap, distillation system, and dropping funnel was added Compound (a-1) (25.00 g, purity 97%, 59.1 mmol) and (R)-2-methylpropane-2-sulfinamide (Compound (A1a) (9.49 g, 78.3 mmol, 1.3 eq.) in MeTHF (200 mL, 8 vol.) under Nitrogen and the resulting yellow cloudy solution was heated at 100° C. Tetraethoxytitanium (100%, 27.50 g, 0.121 mol, 2 eq.) in MeTHF (25 mL, 1 vol.) was added dropwise to the yellow solution via the dropping funnel, and the dropping funnel was rinsed with MeTHF (25 mL, 1 vol.). The reaction mixture was heated under reflux for 4-6 hr by distilling MeTHF to residual volume and refilling the reactor at 10 volumes with fresh MeTHF until conversion reached ˜96.4%-97%. Once the reaction was complete, the reaction mixture was cooled to 20-25° C. and water (17.5 mL, 8 eq. relative to Ti(OEt)₄) was added to the orange solution. The obtained suspension was filtered over a pad of Celite® and the cake was washed with MeTHF (4*5 vol). The yellow filtrate was kept as a MeTHE solution to afford Compound (a-2a) and the corresponding isopropyl ester (Compound (a-2a′)) in 93.4% yield as determined by HPLC analysis. LCMS analysis showed peaks at 514 (Compound (a-2a)) and 528 (Compound (a-2a′)). Certain parameters are summarized in Table 4.

TABLE 4

| Quantity | | | | |
|---|---|---|---|---|
| of MeTHF | | | | |
| solution | HPLC Assay | LCMS Purity | KF | Chiral Purity |
| 567.4 g | 4.45% a-2a | 77.57% a-2a | 1.60% | 91.3% a-2a |
| | 0.58% a-2a′ | 7.07% a-2a′ | | 7.7% a-2a′ |
| | | | | 0.2% of 3S, |
| | | | | S-sulfinimine*, |
| | | | | 0.8% of 3R, |
| | | | | R-sulfinimine* |

*presence of diastereomer is related to the chiral purity of Compound (a-1) and Compound (A1a)

Compound (a-2a): ¹H NMR (DMSO-d₆, 500 MHZ): δ (ppm) 4.88 (q, J=6.4 Hz, 1H), 4.31 (q, J=7.1 Hz, 2H), 3.98 (q, J=9.5 Hz, 2H), 3.68-3.90 (m, 2H), 3.02-3.21 (m, 2H), 2.48 (s, 3H), 1.56-1.87 (m, 4H), 1.42 (d, J=6.8 Hz, 3H), 1.27-1.33 (m, 3H), 1.17 (s, 9H).

Compound (a-2a′): ¹H NMR (500 MHZ, DMSO-d₆) δ 5.18-5.11 (m, 1H), 5.11-5.03 (m, 1H), 4.05-3.87 (m, 2H), 3.85-3.70 (m, 2H), 3.23-3.11 (m, 2H), 2.49 (br s, 3H), 1.95-1.58 (m, 4H), 1.40-1.36 (m, 3H), 1.36-1.31 (m, 6H), 1.21 (s, 9H).

Mixture of Compound (a-2a)+−10% Compound (a-2a′): ¹H NMR (DMSO-d₆, 500 MHz): δ (ppm) 5.05-5.14 (m, 1H), 4.31 (q, J=7.3 Hz, 2H), 3.87-4.00 (m, 2H), 3.71-3.83 (m, 2H), 3.07-3.19 (m, 2H), 2.48 (s, 3H), 1.72-1.82 (m, 2H), 1.60-1.70 (m, 2H), 1.35 (d, J=6.8 Hz, 3H), 1.28-1.32 (m, 3H), 1.18 (s, 9H).

Step 2. Preparation of Compound (a-5a). To a 1 L, 4-neck, round-bottom flask equipped with thermometer, bleach trap, distillation system, and peristaltic pump was added a MeTHE solution of 567.4 g containing Compound (a-2a) (4.5% w/w, 49.0 mmol) and Compound (a-2a′) (0.58% w/w, 6.18 mmol) under N₂. The solution was azeotroped twice and concentrated at 100° C. to reach an equivalent of 10% w/w MeTHF solution a water content around 0.30%. The reaction mixture was then cooled down to −20° C. and 1 M diisobutylaluminum hydride (331 mL, 0.331 mol, 6 eq., calculated relative to both Compounds (a-2a) and (a-2a′)) was added dropwise to the yellow solution. Upon completion of the reaction (˜2 h after the end of addition), the reaction mixture was quenched at −20° C. with the controlled addition of 331 mL of MeOH; then 13.8 mL of water; 13.8 mL of sodium hydroxide (15% w/w); and 33.1 mL of water. The reaction mixture was warmed to 20-25° C. and stirred over 3-4 h. The suspension obtained was filtered and the cake was washed with MeTHF (3*5 vol). The resulting yellow filtrate was kept as a MeTHE solution. Compound (a-5a) was obtained in 80.5% yield, as determined by HPLC assay analysis of MeTHE solution. LCMS analysis showed a peak at 474 (Compound (a-5a)). Certain parameters are summarized in Table 5.

TABLE 5

| Quantity | HPLC Assay | LCMS Purity | Chiral Purity |
|---|---|---|---|
| 773.4 g | 2.73% a-5a | 67.75% a-5a | 97.3% a-5a (3S,4S and |
| | | | R-terbutanesulfinamide) |
| | | | 0.8% of 3S,4R, |
| | | | S-terbutanesulfinamide, 1.2% |
| | | | of 3S,4S, |
| | | | S-terbutanesulfinamide, |
| | | | 0.4% of 3R,4R, |
| | | | R-terbutanesulfinamide |

Compound (a-5a): ¹H NMR (500 MHZ, DMSO-d₆, 300 K) 8 ppm 5.45 (t, J=5.9 Hz, 1 H), 5.09 (d, J=11.0 Hz, 1H), 4.42 (d, J=5.9 Hz, 2H), 4.12 (quin, J=6.4 Hz, 1H), 3.81 (d, J=8.6 Hz, 1H), 3.49 (d, J=8.8 Hz, 2H), 3.46-3.64 (m, 1H), 3.41 (dd, J=10.8, 6.1 Hz, 1H), 2.85-3.02 (m, 2H), 2.45 (s, 3H), 1.74-1.99 (m, 2H), 1.49-1.69 (m, 2H), 1.16 (s, 9H), 1.09 (d, J=6.6 Hz, 3H).

Step 3. Preparation of Compound (a-6). To a 500 mL, 4-neck, round-bottom flask equipped with thermometer, bleach trap, distillation system, and dropping funnel was added a 773.4 g of a MeTHF solution containing 2.7% w/w of Compound (a-5a) (44.4 mmol) under N₂. The solution was distilled at 100° C. to obtain a residual 210 mL of MeTHE solution. Water (53 mL, 2.5 vol) was added and the reaction mixture was cooled down to 0-5° C. Aqueous concentrated hydrogen chloride (36%, 40 mL, 0.444 mol, 10 eq.) was added dropwise. At the end of the addition, the temperature was increased to 20-25° C. After 2 h of stirring, the reaction was complete.

The acidic aqueous layer was separated from the MeTHF layer, cooled down to 0-5° C. and residual MeTHF was removed under a strong bubbling of N₂. Then sodium hydroxide (30%) was added to reach a pH of 12. The reaction mixture was warmed to 20-25° C. and precipitation occurs. The solid was filtered and rinsed with water (3*5 vol) to afford Compound (a-6) with 82.4% yield. LCMS analysis showed a peak at 370 (Compound (a-6)). Certain parameters are summarized in Table 6.

TABLE 6

| Quantity | HPLC Assay | LCMS Purity | Chiral Purity |
|---|---|---|---|
| 15.51 g | 87.68% a-6 | 89.82% a-6 | 98.4% of a-6 |
| | | | 0.6% (3S,4R dia) |
| | | | 1.0% (3R,4S dia) |

Compound (a-6): ¹H NMR (DMSO-d₆, 600 MHZ) δ 5.42 (br t, 1H, J=5.7 Hz), 4.42 (d, 2H, J=5.3 Hz), 3.9-4.1 (m, 1H), 3.63 (d, 1H, J=8.5 Hz), 3.46 (d, 3H, J=8.4 Hz), 3.0-3.2 (m, 2H), 2.87 (d, 1H, J=5.1 Hz), 2.44 (s, 3H), 1.80 (ddd, 1H, J=3.5, 9.6, 13.3 Hz), 1.69 (ddd, 1H, J=3.7, 9.5, 13.3 Hz), 1.5-1.6 (m, 2H), 1.29 (br d, 2H, J=1.2 Hz), 1.07 (d, 3H, J=6.5 Hz).

### Example 17. Preparation of Compound (11)

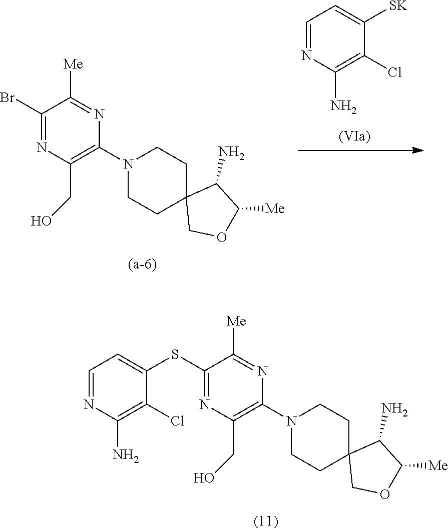

Compound (11), or a salt thereof, was prepared by coupling Compound (a-6) with Compound (VIa).

A 5 L flask was charged with Compound (a-6) (100 g, 1.0 equiv.), Compound (VIa) (64.22 g, 1.2 equiv.), CuI (10 g, 0.2 equiv.), and pyridine (980 g). The mixture was de-gassed with N₂ and heated to 110-120° C. for 20-30 h. The reaction mixture was cooled to 20-30° C. and filtered through diatomite. The cake was washed with pyridine (400 g) and the filtrate was reduced under vacuum. Next, MeOH (237 g) and DCM (3990 g) were added to the filtrate and stirred. To the resulting solution was added NH₃·H₂O (10 wt. %, 1000 g) and the reaction mixture was stirred at 20-30° C. for 1-2 h. The aqueous phase was washed with DCM. To the combined organic phases was added MeOH (158 g) and the organic phase was filtered via 0.45 μm filter. The 0.45 μm filter was washed with DCM/MeOH (4:1 v/v) and the organic phase was concentrated under vacuum. DCM/MeOH (4:1 v/v) was added, followed by dropwise addition of MTBE (111 g) over 1 h. Additional MTBE (703 g) was added over about 6 h, and the reaction mixture was stirred for 8-24 h at 20-30° C. The mixture was filtered and the cake was dried to afford Compound (11) in 55-85% yield.

### Example 18. Preparation of 3-Chloro-2-(methylamine)pyridine-4-thiol Potassium Salt (Compound (VIa))

Route A. Compound (VIa) was prepared according to the reactions shown below:

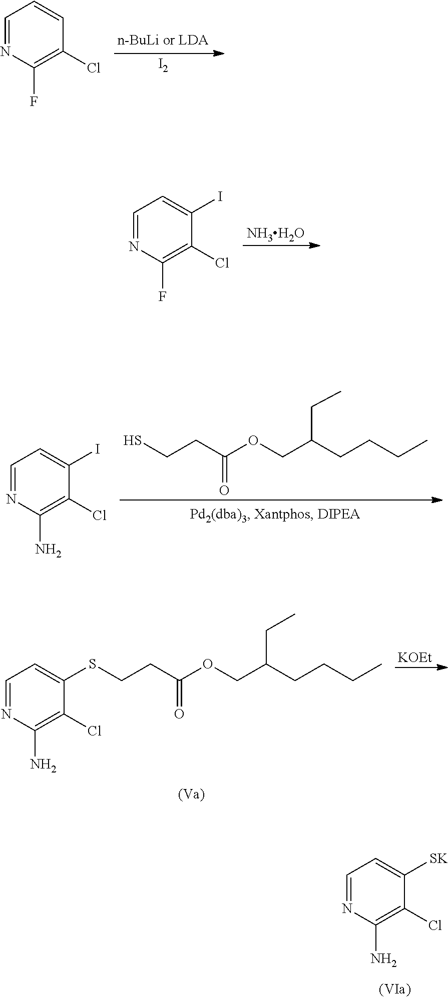

### Step 1.

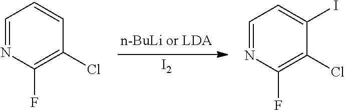 [SMILES: Fc1ncccc1Cl ; "I2" ; Fc1nccc(I)c1Cl]

To a 500 L reactor was charged 2-MeTHF (50 mL) and degassed with nitrogen. The mixture was charged LDA (2.0 M, 102.6 L, 1.2 equiv), then cooled to −70 to −60° C. To the mixture was added a solution of 3-Chloro-2-fluoropyridine (22.5 kg, 171 mol) in 2-MeTHF (40 kg) at −70 to −60° C. The mixture was stirred at −70 to −60° C. for 30 minutes. To the mixture was added a solution of I₂ (47.7 kg, 1.1 equiv) in 2-MeTHF (80 mL) at −70 to −60° C. over 30 minutes. The mixture was stirred at −70 to −60° C. for 60 minutes. TLC (PE:EA=10:1, Rf=0.5) showed full conversion. The reaction mixture was added to another reactor contained 225 kg 1 N HCl solution at 0-20° C. The phases were separated, and the aqueous phase was extracted with EA (79 kg). The combined organic phase was washed with 112.5 kg 30% Na₂S₂O₃ solution. The organic phase was dried over anhydrous MgSO₄ (45 kg), filtered. The cake was rinsed with EA (11.25 kg). The combined filtrate was concentrated under reduced to pressure to about 2 vol. To the mixture was added 45 kg MTBE then distilled under reduced pressure at 45±5° C. to about 2 vol, which was repeated for 3 times. The mixture was cooled to 20±5° C. and filtered after 1 hour. The cake was rinsed with 5.6 kg MTBE. The solid was dried under reduced pressure at 45±5° C.° C. to give 3-chloro-2-fluoro-4-iodopyridine (32.0 kg, 99.6% purity, 72.7% yield).

### Step 2.

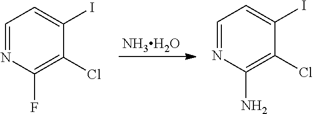

To a 100 L pressure reaction was added 3-chloro-2-fluoro-4-iodopyridine (11.0 kg, 42.7 mol) and ammonium hydroxide solution (33 kg). DMSO (36.3 kg) was slowly added to the mixture. The mixture was warmed to 80±5° C. and stirred for 5 hours. TLC (PE:EA=2:1, Rf=0.3) showed full conversion. After being cooled to 25±5° C., the mixture was added to water (110 kg) in a 500 L reactor, and stirred at 25±5° C. for 1 hour. The mixture was filtered. The cake was reslurried two times water (55 kg×2) at 20±5° C. The cake was dried under reduced pressure at 45±5° C.° C. to give 3-Chloro-4-iodo-2-pyridinamine (30.8 kg, 99.3% purity, 94.4% yield) as a solid.

### Step 3. Synthesis of (Va).

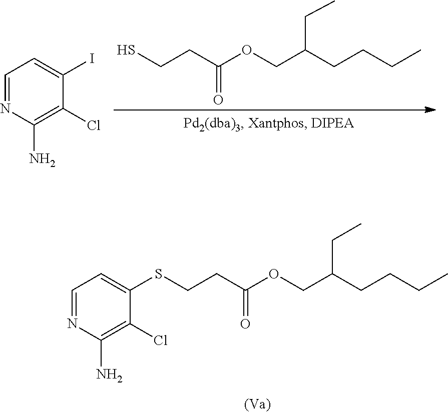

To a 1000 L reactor was added dioxane (121 kg), 3-chloro-4-iodo-2-pyridinamine (23.4 kg, 92.0 mol), 2-Ethylhexyl 3-mercaptopropionate (21.1 kg, 96.6 mol, 1.05 equiv), DIPEA (23.8 kg, 2 equiv), Xantphos (0.267 kg, 0.005 equiv), and Pd(OAc)₂ (0.103 kg, 0.005 equiv). After being purged with nitrogen for three times, the mixture was warmed to 95±5° C. and stirred for 5 hours. TLC (PE:EA=2:1, Rf=0.3) showed full conversion. After cooled to 20±5° C., the mixture was added 2-MeTHF (187 kg) and water (117 kg), and stirred for 30 minutes. After phase separation, the organic phase was washed with water (117 kg). The combined aqueous phase was extracted with 2-MeTHF (47 kg). The combined organic phase was dried over anhydrous MgSO₄ (37 kg) and filtered through a pad of silica gel (100-200 mesh, 9.5 kg). The silica gel pad was rinsed with MeTHF (70 kg×2). The combined filtrate was distilled under reduced pressure to about 2 vol. The mixture was added EA (47 kg) and distilled under reduced pressure at 45±5° C. to about 2 vol. The mixture was added EA (47 kg) and distilled under reduced pressure at 45±5° C. to about 2 vol. The mixture was added EA (84 kg) and distilled under reduced pressure at 45±5° C. to about 2.5 vol. The mixture was cooled to 15±5° C. After stirred for 1 hour, the mixture was filtered and rinsed with 9 kg EA. The cake was dried at 45±5° C. to give 2-ethylhexyl 3-((2-amino-3-chloropyridin-4-yl)thio) propanoate (Va) (28.6 kg, 97.9% purity, 90.2% yield).

### Step 4.

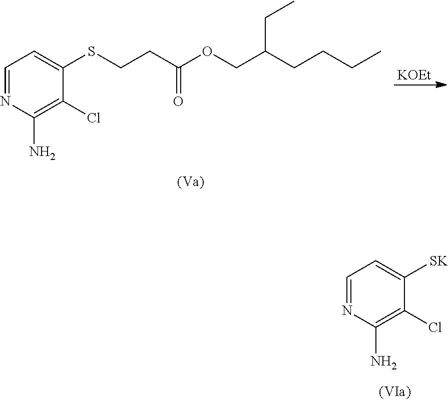

To a reactor was charged 2-ethylhexyl 3-((2-amino-3-chloropyridin-4-yl)thio) propanoate (Va) (38.1 kg, 1 equiv) and 2-MeTHF (312 kg). The resulting mixture was maintained at 15-25° C. for 2 hour at which point a clear solution formed. To this was charged KOEt (˜24% w/w in EtOH) (41.8 kg, 1.1 equiv) over two hours, maintaining a temperature of 15-25° C., followed by additional 2-MeTHF (4 kg). The reaction was maintained at 15-25° C. for 4 hours at which point UPLC monitoring showed reaction completion.

To the reactor was charged MTBE (147 kg). The resulting suspension was maintained at 15-25° C. for 4 hours and then filtered, washing the cake with more MTBE (84 kg). The cake was dried for 24 hours at ≤30° C. to afford potassium 2-amino-3-chloropyridine-4-thiolate (VIa) (21.65 kg, 96.9% a/a UPLC purity, 95.8% w/w assay, 95% assay-corrected yield).

Route B. Compound (VIa) was prepared according to the scheme shown below:

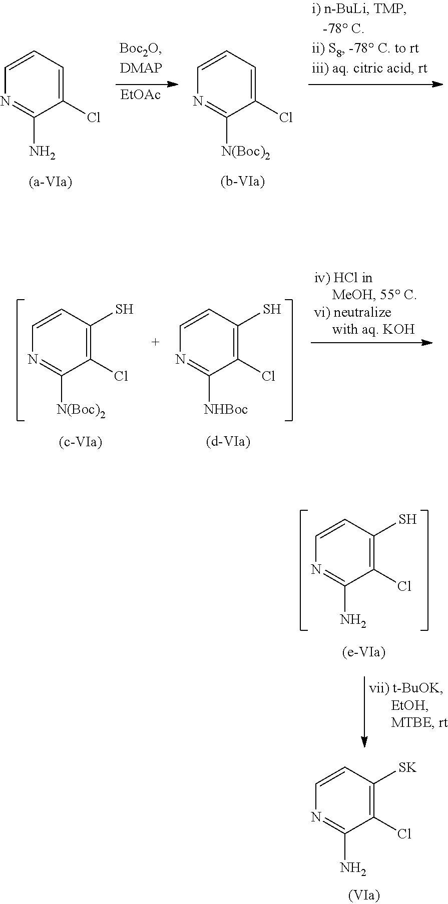

Step 1. Synthesis of (b-VIa).

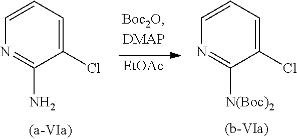

A flask was charged with 3-chloropyridin-2-amine (a-VIa) (100 g, 1.0 equiv), DMAP (5.6 g, 0.1 equiv), and EtOAc (700 mL, 7.0 vol). The resulting mixture was maintained at 25-30° C. for 10 minutes. To this was charged Boc₂O (424 g, 2.5 equiv) over ˜1 hour, maintaining a temperature of 25-30° C., during which time a lot of gas was released. The resulting mixture was maintained at 25-30° C. for 7 hours at which point HPLC monitoring showed reaction completion.

To the reaction was charged 10% w/w aq. citric acid (200 mL, 2.0 vol) and the resulting mixture was maintained at 25-30° C. for 10 minutes. The layers were separated and the aqueous layer was extracted with EtOAc (300 mL, 3.0 vol). The combined organic layers were washed with 10% w/w aq. NaCl (200 mL, 2.0 vol) and then concentrated to ˜2 vol under reduced pressure at 45° C. To the concentrate was added n-heptane (300 mL, 3.0 vol) during which time a lot of solid precipitated. The suspension was concentrated to ˜2 vol under reduced pressure at 45° C. To the concentrate was added n-heptane (200 mL, 2.0 vol) and the resulting mixture was maintained at 25-30° C. for 30 minutes and then filtered, washing the cake with n-heptane (100 mL, 1.0 vol). The cake was dried under reduced pressure at 45° C. for 4 h to afford tert-butyl (tert-butoxycarbonyl) (3-chloropyridin-2-yl) carbamate (b-VIa) (225 g, 98.3% a/a HPLC purity, 86% corrected yield).

¹H NMR (400 MHZ, CDCl₃) δ 8.43 (dd, J=4, 8 Hz, 1H), 7.80 (dd, J=4, 8 Hz, 1H), 7.26 (dd, J=4, 8 Hz, 1H), 1.41 (s, 18H).

MS (ESI+): calculated, 329.13; found, 329.1.

### Step 2. Synthesis of (VIa).

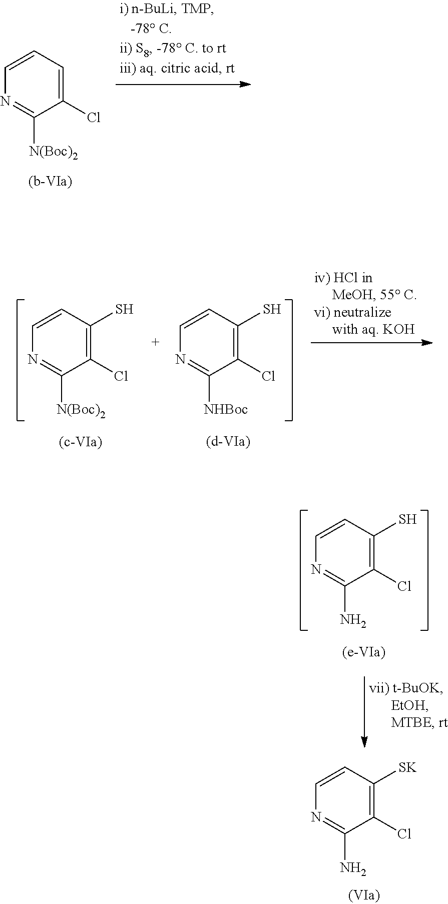

To a flask was charged 2,2,6,6-tetramethylpiperidine (TMP) (103 g, 1.5 equiv) and THF (1.6 L, 10 vol). The flask was evacuated and backfilled with nitrogen 3 times. The solution was cooled to −80 to −70° C. To this was charged n-BuLi (311 mL, 1.5 equiv, 2.5 mol/L), maintaining a temperature of −80 to −70° C. The reaction was maintained at −80 to −70° C. for 30 minutes. To this was charged a solution of tert-butyl (tert-butoxycarbonyl) (3-chloropyridin-2-yl) carbamate (b-VIa) (160 g, 1.0 equiv) in THF (800 mL, 5 vol) over 60 minutes, maintaining a temperature of −80 to −70° C. The resulting mixture was maintained at −80 to −70° C. for 2 hours. To this was charged S₈ (23.4 g, 1.5 equiv), maintaining a temperature of −80 to −70° C. The resulting mixture was warmed to 20-30° C. and maintained at 20-25° C. for 2 hours at which point HPLC monitoring showed reaction completion.

The reaction was quenched with water (800 mL, 5 vol), maintaining a temperature of 20-30° C. The mixture was extracted with MTBE (1.6 L, 10 vol) and the organic layer was washed with water (800 mL, 5 vol). To the combined aqueous layers was added 25% w/w aq. Na₂SO₃ (1.6 L, 10 vol), maintaining a temperature of 20-30° C. The solution was maintained at 20-30° C. for 3 hours. The pH was then adjusted to 5-6 with 30% w/w aq. citric acid (1.2 L, 7.5 vol). This was then extracted with 2-MeTHF (800 mL×3). The combined organic layers were washed with 20% w/w aq. NaCl (800 mL, 5 vol).

To the reaction mixture was charged 36% w/w HCl in MeOH (395 g, 8.0 equiv). The resulting mixture was maintained at 50-60° C. for 4 hours during which time white solid precipitated and HPLC monitoring showed reaction completion.

The MeOH and 2-MeTHF were removed by distillation under reduced pressure at ca. 45° C. To the resulting residue was added water (320 mL, 2.0 vol). This was concentrated to ˜2 vol under reduced pressure at 50-55° C. The pH was adjusted to 7 with 40% w/w aq. KOH (ca. 280 mL). The resulting suspension was filtered, washing the cake with water (160 mL). The cake was dried under reduced pressure at 55° C. for 4 hours to afford 2-amino-3-chloropyridine-4-thiol (e-VIa) (64.6 g, 94.6% HPLC purity) that was used directly in next step.

The crude 2-amino-3-chloropyridine-4-thiol (e-VIa) was suspended in 2-MeTHF (260 mL, 4 vol). To the suspension was added 1-BuOK (47.6 g, 1.05 equiv) in EtOH (142 g), maintaining a temperature of 20-30° C., during which time a clear solution formed. This was maintained at 20-30° C. for 30 minutes. To this was charged MTBE (260 mL, 4.0 vol) and the resulting mixture was maintained for 30 minutes until precipitation had stopped. To this was then charged additional MTBE (710 mL, 11 vol) and the resulting suspension was maintained for 60 minutes. The suspension was then filtered, washing the cake with MTBE (320 mL). The cake was dried under reduced pressure at 45° C. for 4 hours to afford potassium 2-amino-3-chloropyridine-4-thiolate (VIa) (72 g, 98.8% a/a HPLC purity, 74% yield).

¹H NMR for potassium 2-amino-3-chloropyridine-4-thiolate (VIa) (400 MHZ, DMSO-d₆) δ 7.04 (d, J=8 Hz, 1H), 6.47 (d, J=8 Hz, 1H), 5.04 (br s, 2H).

¹³C NMR for potassium 2-amino-3-chloropyridine-4-thiolate (VIa) (100 MHz, DMSO-d₆) δ 169.3, 154.6, 140.8, 122.3, 116.5.

Potassium (K) content for potassium 2-amino-3-chloropyridine-4-thiolate (VIa) (ICP-MS): calculated, 196.76 g/kg; found, 194.33 g/kg.

MS for potassium 2-amino-3-chloropyridine-4-thiolate (VIa) (ESI+): calculated, 160.99; found, 161.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby. The disclosures of all patent and scientific literature cited herein are expressly incorporated herein in their entirety by reference. To the extent that any incorporated material is inconsistent with the express content of this disclosure, the express content controls.

1. A method of preparing a compound of Formula (7): 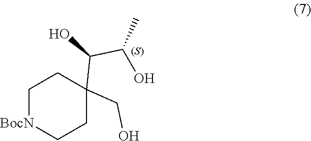
comprising:
reacting a compound of Formula (6): 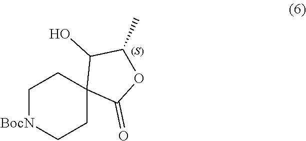
with a reducing agent to form the compound of Formula (7).

2. The method of claim 1, wherein the reducing agent is an organoaluminum hydride, an aluminum hydride, an organoborane hydride, or a borohydride reagent.

3. The method of claim 1 or 2, wherein the reaction is carried out using an alcohol, an aprotic solvent, or a mixture thereof as solvent.

4. A method of preparing a compound of Formula (6): 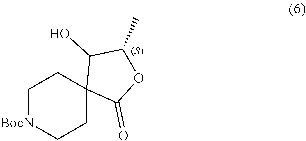
comprising reacting a compound of Formula (5): 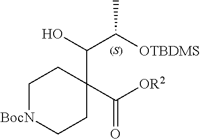
wherein R² is C₁-C₆ alkyl, and TBDMS is tert-butyldimethylsilyl ether,
with a deprotecting agent to form the compound of Formula (6).

5. The method of any one of claims 1-4, wherein the compound of Formula (6) is prepared by reacting a compound of Formula (5): 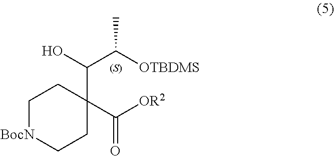
wherein R² is C₁-C₆ alkyl, and TBDMS is tert-butyldimethylsilyl ether,
with a deprotecting agent to form the compound of Formula (6).

6. The method of claim 4 or 5, wherein the deprotecting agent is a source of fluoride ion, acetyl chloride, N-iodosuccinimide, HCl, acetic acid, formic acid, phosphoric acid, FeCl₃, AlCl₃, CeCl₃, oxalyl chloride, isobutyl chloroformate, ethyl chloroformate, or thionyl chloride.

7. The method of any one of claims 4-6, wherein the reaction of the compound of Formula (5) with the deprotecting agent is carried out using an aprotic solvent.

8. The method of any one of claims 4-7, wherein the compound of Formula (5) is prepared by reacting a compound of Formula (3): 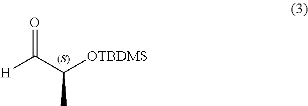
with a compound of Formula (4): 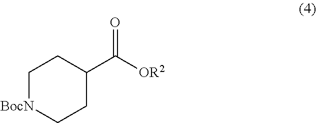
wherein R² is C₁-C₆ alkyl,
to form the compound of Formula (5).

9. The method of claim 8, wherein the reaction of the compound of Formula (3) with the compound of Formula (4) further comprises a base.

10. The method of claim 8 or 9, wherein the reaction of the compound of Formula (3) with the compound of Formula (4) is carried out using an aprotic solvent.

11. The method of any one of claims 8-10, wherein the compound of Formula (3) is prepared by reacting a compound of Formula (2): 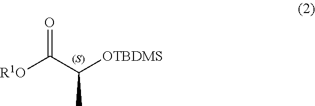
wherein R¹ is C₁-C₆ alkyl,
with a reducing agent to form the compound of Formula (3).

12. The method of claim 11, wherein the reducing agent used for the reaction of the compound of Formula (2) is an organoaluminum hydride, an aluminum hydride, an organoborane hydride, or a borohydride reagent.

13. The method of claim 11 or 12, wherein the reaction of the compound of Formula (2) with the reducing agent is carried out using an aprotic solvent.

14. The method of any one of claims 11-13, wherein the compound of Formula (2) is prepared by reacting a compound of Formula (1): 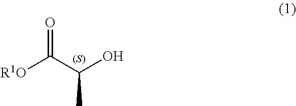
wherein R¹ is C₁-C₆ alkyl,
with TBDMS-X, wherein X is a halide,
to form the compound of Formula (2).

15. The method of claim 14, wherein the reaction of the compound of Formula (1) with TBDMS-X further comprises a base.

16. The method of claim 14 or 15, wherein the reaction of the compound of Formula (1) with TBDMS-X is carried out using an aprotic solvent.

17. A method of preparing a compound of Formula (8): 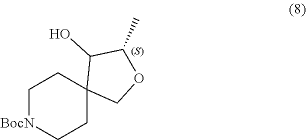
comprising reacting the compound of Formula (7) prepared according to the method of any one of claims 1-16 with a sulfonyl chloride or an acid chloride, and a base to form the compound of Formula (8).

18. The method of claim 17, wherein the sulfonyl chloride or the acid chloride is an arylsulfonyl chloride, an alkylsulfonyl chloride, or an aryl acid chloride.

19. The method of claim 17 or 18, wherein the reaction of Formula (7) with the sulfonyl chloride or the acid chloride and the base is carried out using a mixture of water and an aprotic solvent.

20. A method of preparing a compound of Formula (9): 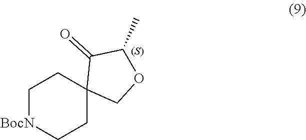
comprising reacting the compound of Formula (8) prepared according to the method of any one of claims 17-19 with an oxidizing agent to form the compound of Formula (9).

21. The method of claim 20, wherein the oxidizing agent is Dess-Martin periodinane, (2,2,6,6-tetramethylpiperidine-1-yl)oxyl (TEMPO), or sulfur trioxide pyridine complex.

22. The method of claim 21, wherein the reaction of the compound of Formula (8) with TEMPO further comprises (diacetoxyiodo)benzene or sodium hypochlorite, and optionally further comprises a salt.

23. The method of any one of claims 20-22, wherein the reaction of the compound of Formula (8) with the oxidizing agent is carried out using an aprotic solvent or a mixture of an aprotic solvent and water.

24. A method of preparing a compound of Formula (10) or a salt thereof: 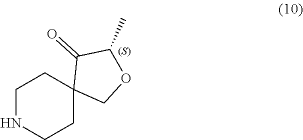
comprising reacting the compound of Formula (9) prepared according to the method of any one of claims 20-23 with an acid to form the compound of Formula (10) or a salt thereof.

25. A method of preparing a compound of Formula (10)⋅HCl comprising the following steps: 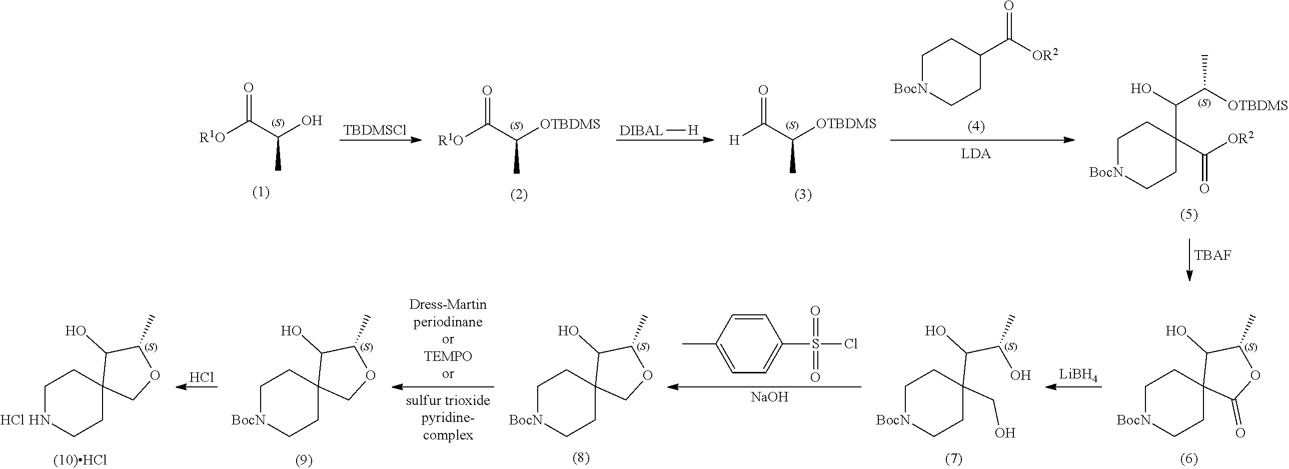
wherein R¹ and R² are each independently methyl or ethyl.

26. The method of any one of claims 20-25, wherein the compound of Formula (9) and/or the compound of Formula (10), or a salt thereof, are prepared without use of column chromatography.

27. A method of preparing a compound of Formula (11): 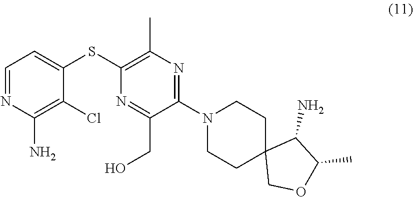 [SMILES: Cc1nc(N2CCC3(CC2)CO[C@@H](C)[C@H]3N)c(CO)nc1Sc1ccnc(N)c1Cl]
or a salt thereof, comprising converting the compound of Formula (10), or a salt thereof, prepared according to any one of claims 24-26 to the compound of Formula (11) or a salt thereof.

28. A compound of Formula (6): [chemical structure]
